(19) United States
(12) Patent Application Publication
(10) Pub. No.: US 2026/0253677 A1
(43) Pub. Date: Aug. 27, 2026

(54) DIAGNOSIS OF PANCREATIC CANCER USING TARGETED QUANTIFICATION OF SITE-SPECIFIC PROTEIN GLYCOSYLATION

(71) Applicant: VENN BIOSCIENCES CORPORATION, South San Francisco, CA (US)

(72) Inventors: Daniel SERIE, South San Francisco, CA (US); Chad Eagle PICKERING, South San Francisco, CA (US); Gege XU, South San Francisco, CA (US)

(21) Appl. No.: 18/714,222

(22) PCT Filed: Nov. 30, 2022
(86) PCT No.: PCT/US2022/080692

§ 371 Date: May 29, 2024

Related U.S. Application Data
(60) Provisional application No. 63/284,594, filed on Nov. 30, 2021.

(51) Int. Cl. G16B 40/20; G01N 33/68; G16B 15/00; G16H 50/20

(52) U.S. Cl. CPC G16B 40/20; G01N 33/6848; G16B 15/00; G16H 50/20

(57) ABSTRACT

A method and system for diagnosing a subject with respect to a pancreatic cancer disease state. Peptide structure data corresponding to a biological sample obtained from the subject is received. The peptide structure data is analyzed using a supervised machine learning model to generate a disease indicator that indicates whether biological sample evidences the PC disease state based on at least 3 peptide structures selected from a group of peptide structures of Group I identified in Table 1 or of Group II of Table 8. The group of peptide structures in Table 1 or Table 8 comprises a group of peptide structures associated with the PC disease state. The group of peptide structures is listed in Table 1 with respect to relative significance to the disease indicator. A diagnosis output is generated based on the disease indicator

### CROSS-REFERENCE TO RELATED ART

This application claims priority to U.S. Provisional Patent Application Ser. No. 63/284,594, filed Nov. 30, 2021, which is incorporated by reference herein in its entirety.

### FIELD

The present disclosure generally relates at least to methods and systems for analyzing peptide structures for diagnosing and/or treating pancreatic cancer. More particularly, the present disclosure relates to analyzing quantification data for a set of peptide structures detected in a biological sample obtained from a subject for use in diagnosing and/or treating the subject, the set of peptide structures being associated with pancreatic cancer.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat pancreatic cancer (PC).

Diagnosing and treating PC currently relies on protein assays evaluated using enzyme-linked immunosorbent assay (ELISA)-based technology. For example, the standard proteins evaluated using ELISA-based technology include the CA 19-9 and CEA proteins. However, evaluations based on these proteins may not provide the level of performance desired with respect to predicting or diagnosing PC. Further, currently available methods for diagnosing PC may be unable to make an early diagnosis of PC. Late diagnosis of PC in patients can lead to negative health outcomes.

An approach that is both non-invasive and includes a low false positive rate while maintaining a high level of accuracy is needed. Additionally, an approach enabling early diagnosis may help reduce negative health outcomes in patients with PC. Thus, it may be desirable to have methods and systems capable of addressing one or more of the above-identified issues.

### SUMMARY

In one aspect, a method for diagnosing a subject with respect to a pancreatic cancer (PC) disease state is described in accordance with various embodiments. In various embodiments, the method includes receiving peptide structure data corresponding to one or more biological samples obtained from the subject, such as one or more liquid biological samples from the subject.

In various embodiments, the present disclosure encompasses generation of diagnosis outputs for a subject using different sets of peptide structure data obtained from the subject. In specific embodiments, methods of the disclosure may utilize analysis of distinctly different sets of peptide structure data that are applied to a set of peptide structure data, including one of two sets of data provided in Tables 1-7C or in Tables 8-14. In various embodiments, the method includes analyzing the peptide structure data using at least one supervised machine learning model to generate a disease indicator that indicates whether the biological sample evidences a PC disease state based on at least 3 peptide structures selected from a group of peptide structures identified in Table 1 or Table 8. In various embodiments, the group of peptide structures in Table 1 or Table 8 is associated with the PC disease state. In various embodiments, the group of peptide structures is listed in Table 1 or Table 8 with respect to relative significance to the disease indicator. In various embodiments, the method includes generating a diagnosis output based on the disease indicator.

In one aspect, a method of training at least one model to diagnose a subject with respect to a pancreatic cancer (PC) disease state is described in accordance with various embodiments. In various embodiments, the method includes receiving quantification data for a panel of peptide structures for a plurality of subjects. In various embodiments, the plurality of subjects includes a first portion diagnosed with a negative diagnosis of a PC disease state and a second portion diagnosed with a positive diagnosis of the PC disease state. In various embodiments, the quantification data comprises a plurality of peptide structure profiles for the plurality of subjects. In various embodiments, the method includes training a machine learning model using the quantification data to diagnose a biological sample with respect to the PC disease state using a group of peptide structures associated with the PC disease state. In various embodiments, the group of peptide structures is identified in Table 1 or Table 8. In various embodiments, the group of peptide structures is listed in Table 1 or Table 8 with respect to relative significance to diagnosing the biological sample.

In one aspect, a method of monitoring a subject for a pancreatic cancer (PC) disease state is described in accordance with various embodiments. In various embodiments, the method includes receiving first peptide structure data for a first biological sample obtained from a subject at a first timepoint. In various embodiments, the method includes analyzing the first peptide structure data using at least one supervised machine learning model to generate a first disease indicator based on at least 3 peptide structures selected from a group of peptide structures identified in Table 1 or Table 8, wherein the group of peptide structures in Table 1 or Table 8 comprises a group of peptide structures associated with a PC disease state. In various embodiments, the method includes receiving second peptide structure data of a second biological sample obtained from the subject at a second timepoint. In various embodiments, the method includes analyzing the second peptide structure data using the supervised machine learning model to generate a second disease indicator based on the at least 3 peptide structures selected from the group of peptide structures identified in Table 1 or Table 8. In various embodiments, the method includes generating a diagnosis output based on the first disease indicator and the second disease indicator. In some embodiments, the method encompasses monitoring a subject for progression of the disease, whereas in other embodiments the method encompasses monitoring a state of the disease before and after administering at least one treatment using one or more therapies for the disease.

In one aspect, a composition comprising at least one of peptide structures PS-1 to PS-38 identified in Table 1 with respect to a first group of peptide structures is described according to various embodiments. In one aspect, a composition comprising at least one of peptide structures PS-1 to PS-5, PS-8, PS-9, PS-12 to PS-15, PS-17, PS-20, PS-26, and PS-33 to PS-38 identified in Table 2 also with respect to a first group of peptide structures is described according to various embodiments. In one aspect, a composition comprising at least one of peptide structures PS-1 to PS-22 identified in Table 8 with respect to a second group of peptide structures is described, according to various embodiments.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 18-40, corresponding to peptide structures PS-1 to PS-38 in Table 1. In various embodiments, the product ion is selected as one from a group consisting of product ions identified in Table 3 including product ions falling within an identified m/z range. In various embodiments, the peptide structure or the product ion comprises an amino acid sequence having at least 90% sequence identity to any one of SEQ ID NOS: 18, 21, 25, 28, 32, 51-67, corresponding to peptide structures PS-1 to PS-22 in Table 8. In various embodiments, the product ion is selected as one from a group consisting of product ions identified in Table 10 including product ions falling within an identified m/z range.

In one aspect, a composition comprising a glycopeptide structure selected as one from a group consisting of peptide structures PS-1 to PS-38 identified in Table 1 according to various embodiments. In various embodiments, the glycopeptide structure comprises an amino acid peptide sequence identified in Table 4 as corresponding to the glycopeptide structure and a glycan structure identified in Table 6 as corresponding to the glycopeptide structure in which the glycan structure is linked to a residue of the amino acid peptide sequence at a corresponding position identified in Table 1. In various embodiments, the glycan structure has a glycan composition. In one aspect, a composition comprising a glycopeptide structure selected as one from a group consisting of peptide structures PS-1 to PS-22 identified in Table 8 according to various embodiments. In various embodiments, the glycopeptide structure comprises an amino acid peptide sequence identified in Table 11 as corresponding to the glycopeptide structure and a glycan structure identified in Table 13 as corresponding to the glycopeptide structure in which the glycan structure is linked to a residue of the amino acid peptide sequence at a corresponding position identified in Table 8. In various embodiments, the glycan structure has a glycan composition.

In one aspect, a composition comprising a peptide structure selected as one from a plurality of peptide structures identified in Table 1 according to various embodiments. In various embodiments, the peptide structure has a monoisotopic mass identified as corresponding to the peptide structure in Table 1. In various embodiments, the peptide structure comprises the amino acid sequence of SEQ ID NOs: 18-40 identified in Table 1 as corresponding to the peptide structure. In one aspect, a composition comprising a peptide structure selected as one from a plurality of peptide structures identified in Table 8 according to various embodiments. In various embodiments, the peptide structure has a monoisotopic mass identified as corresponding to the peptide structure in Table 8. In various embodiments, the peptide structure comprises the amino acid sequence of SEQ ID NOs: 18, 21, 25, 28, 32, 51-67 identified in Table 8 as corresponding to the peptide structure.

In one aspect, a composition comprising at least one of peptide structures PS-1 to PS-8, PS-10 to PS-14, PS-16 to PS-19, PS-21 to PS-25, PS-28 to PS-29, PS-31 to PS-34, PS-36 to PS-38 identified in Table 1 is described according to various embodiments. In one aspect, a composition comprising at least one of peptide structures PS-1 to PS-22 identified in Table 8 is described according to various embodiments.

In one aspect, a composition comprising a peptide structure or a product ion is described according to various embodiments. In various embodiments, the peptide structure or the product ion comprises an amino acid sequence having at least 90% sequence identity to any one of SEQ ID NOS: 18-23, 25-28, 30-32, 35-36, and 38-40. In various embodiments, the product ion is selected as one from a group consisting of product ions identified in Table 3 including product ions falling within an identified m/z range. In one aspect, a composition comprising a peptide structure or a product ion is described according to various embodiments. In various embodiments, the peptide structure or the product ion comprises an amino acid sequence having at least 90% sequence identity to any one of SEQ ID NOS: 18, 21, 25, 28, 32, 51-67. In various embodiments, the product ion is selected as one from a group consisting of product ions identified in Table 10 including product ions falling within an identified m/z range.

In one aspect, a composition comprising a glycopeptide structure selected as one from a group consisting of peptide structures PS-1 to PS-8, PS-10 to PS-14, PS-16 to PS-19, PS-21 to PS-25, PS-28 to PS-29, PS-31 to PS-34, PS-36 to PS-38 identified in Table 1 is described according to various embodiments. In various embodiments, the glycopeptide structure comprises an amino acid peptide sequence identified in Table 4 as corresponding to the glycopeptide structure. In various embodiments, a glycan structure identified in Table 6 as corresponding to the glycopeptide structure in which the glycan structure is linked to a residue of the amino acid peptide sequence at a corresponding position identified in Table 1. In various embodiments, the glycan structure has a glycan composition. In one aspect, a composition comprising a glycopeptide structure selected as one from a group consisting of peptide structures PS-1 to PS-22 identified in Table 8 is described according to various embodiments. In various embodiments, the glycopeptide structure comprises an amino acid peptide sequence identified in Table 11 as corresponding to the glycopeptide structure. In various embodiments, a glycan structure identified in Table 13 as corresponding to the glycopeptide structure in which the glycan structure is linked to a residue of the amino acid peptide sequence at a corresponding position identified in Table 8. In various embodiments, the glycan structure has a glycan composition.

In one aspect, a composition comprising a peptide structure selected as one of PS-1 to PS-8, PS-10 to PS-14, PS-16 to PS-19, PS-21 to PS-25, PS-28 to PS-29, PS-31 to PS-34, PS-36 to PS-38 peptide structures identified in Table 1 is described according to various embodiments. In various embodiments, the peptide structure has a monoisotopic mass identified as corresponding to the peptide structure in Table 1. In various embodiments, the peptide structure comprises the amino acid sequence of SEQ ID NOS: 18-23, 25-28, 30-32, 35-36, and 38-40 identified in Table 1 as corresponding to the peptide structure. In one aspect, a composition comprising a peptide structure selected as one of PS-1 to PS-22 peptide structures identified in Table 8 is described according to various embodiments. In various embodiments, the peptide structure has a monoisotopic mass identified as corresponding to the peptide structure in Table 8. In various embodiments, the peptide structure comprises the amino acid sequence of SEQ ID NOs: 18, 21, 25, 28, 32, 51-67 identified in Table 8 as corresponding to the peptide structure.

In one aspect, a kit comprising at least one agent for quantifying at least one peptide structure identified in Table 1 or Table 8 to carry out part or all of any one or more of the methods described herein.

In one aspect, a kit comprising at least one agent for quantifying at least one peptide structure identified in Table 2 or Table 9 to carry out part or all of any one or more of the methods described herein.

In one aspect, a kit comprising at least one of a glycopeptide standard, a buffer, or a set of peptide sequences to carry out part or all of any one or more of the methods described herein, a peptide sequence of the set of peptide sequences identified by a corresponding one of SEQ ID NOS: 18-40, defined in Table 1 is described according to various embodiments. In one aspect, a kit comprising at least one of a glycopeptide standard, a buffer, or a set of peptide sequences to carry out part or all of any one or more of the methods described herein, a peptide sequence of the set of peptide sequences identified by a corresponding one of SEQ ID NOS: 18, 21, 25, 28, 32, 51-67, defined in Table 8 is described according to various embodiments.

In one aspect, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one aspect, a computer-program product tangibly embodied in a non-transitory machine-readable storage medium, including instructions configured to cause one or more data processors to perform part or all of any one or more of the methods described herein.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for diagnosing a subject with respect to a pancreatic cancer (PC) disease state in accordance with one or more embodiments.

FIG. 6 is a flowchart of a process for training a model to diagnose a subject with respect to pancreatic cancer (PC) disease state in accordance with one or more embodiments.

FIG. 7 is a flowchart of a process for monitoring a subject for a pancreatic cancer (PC) in accordance with one or more embodiments.

FIG. 8 is a training confusion matrix showing predictive accuracy in accordance with one or more embodiments.

FIG. 9 is a test confusion matrix showing predictive accuracy in accordance with one or more embodiments.

FIG. 10 is a table showing performance metrics for the training and testing cohorts overall and by stage in accordance with one or more embodiments.

FIG. 11 is a table showing performance metrics for the training and testing cohorts by stage in accordance with various embodiments.

FIG. 12 is a receiver operating characteristic (ROC) curve in accordance with one or more embodiments.

FIG. 13 is a clustered heat map comparing z-score values for various biomarkers across patent data set, in accordance with one or more embodiments.

FIG. 14 is a probability dotplot illustrating probabilities of pancreatic cancer across training and test data across various health states, in accordance with one or more embodiments.

FIG. 15 is a probability dotplot illustrating probabilities of pancreatic cancer across training and test data across various health states, in accordance with one or more embodiments.

FIG. 16 is a receiver operating characteristic (ROC) curve in accordance with various embodiments.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to diagnose certain diseases, such as pancreatic cancer (PC), more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., a pancreatic cancer (PC) disease state). This information can be used to distinguish the disease state from other states, diagnose a subject as having or not having the disease state, determine a likelihood that a subject has the disease state, determine a risk for a subject to have the disease state, e.g., compared to the general population, or a combination thereof. For example, such analysis may be useful in diagnosing a PC disease state for a subject (e.g., a negative diagnosis for the PC disease state or a positive diagnosis for the PC disease state). Sample collection and analysis can be collected at different time points for comparing PC disease states over time for a subject, such as monitoring progression of the disease or monitoring efficacy of one or more therapies for the disease. For example, the negative diagnosis may include a healthy state, a benign pancreatitis state (i.e. “benign” as seen throughout), and/or a control state. An example of the positive diagnosis includes the subject suffering from a form of pancreatic cancer (e.g., pancreatic adenocarcinoma). A diagnosis can also assess a malignancy status of a mass previously identified on a subject's pancreas.

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing a PC disease state. A PC disease state may include any condition that can be diagnosed as cancer that occurs in the pancreas. This includes (1) exocrine pancreatic cancer, which includes pancreatic adenocarcinoma, squamous cell carcinoma, adenosquamous carcinoma, and colloid carcinoma; and (2) neuroendocrine pancreatic cancer (also referred to as islet cell tumors) . . . . Further, certain peptide structures that are associated with a PC disease state may be more relevant to that disease state than other peptide structures that are also associated with that disease state.

Analyzing the abundance of peptide sequences and glycosylated peptide sequences in a biological sample may provide a more accurate way in which to distinguish a positive PC disease state (e.g., a state including the presence of pancreatic cancer) from a negative PC disease state (e.g., healthy state, control state, an absence of pancreatic cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

The description below provides exemplary implementations of the methods and systems described herein for the research, diagnosis, and/or treatment of a PC disease state. Various examples implement the methods and systems described herein as a screening tool. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g., —NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject, such as a biopsy that may be solid or liquid. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refer to breaking apart a polymer (e.g., cutting a polypeptide at a cut site). Proteins may be digested in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refer to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein. A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.”

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ(H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses).

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to an amount of a particular peptide structure (e.g., biomarker) present in a biological sample. In some embodiments, the amount may be in relation to other structures present in the sample (e.g., relative abundance). In some embodiments, the quantitative value may comprise an amount of an ion produced using mass spectrometry. In some embodiments, the quantitative value may be associated with an m/z value (e.g., abundance on x-axis and m/z on y-axis). In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### III. Overview of Exemplary Workflow

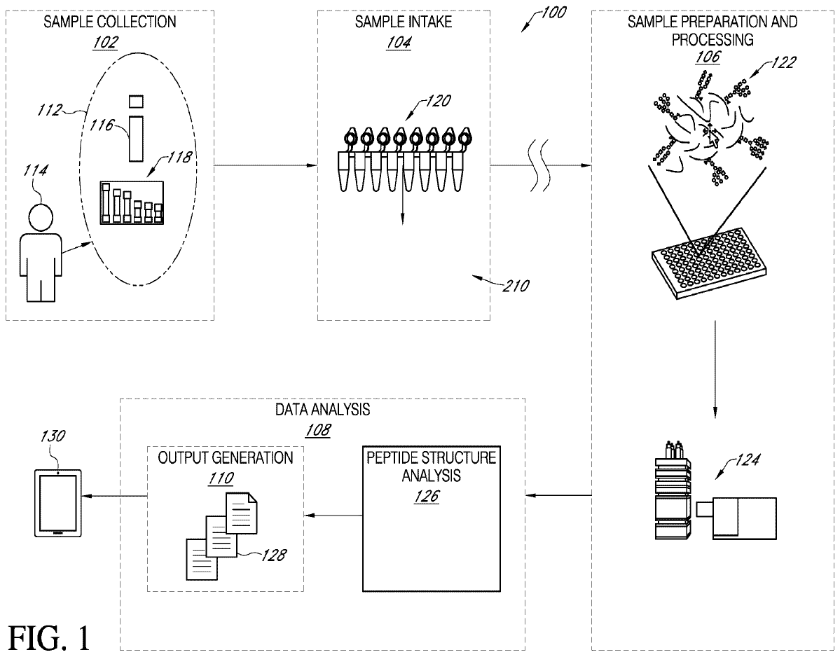

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### IV. Detection and Quantification of Peptide Structures

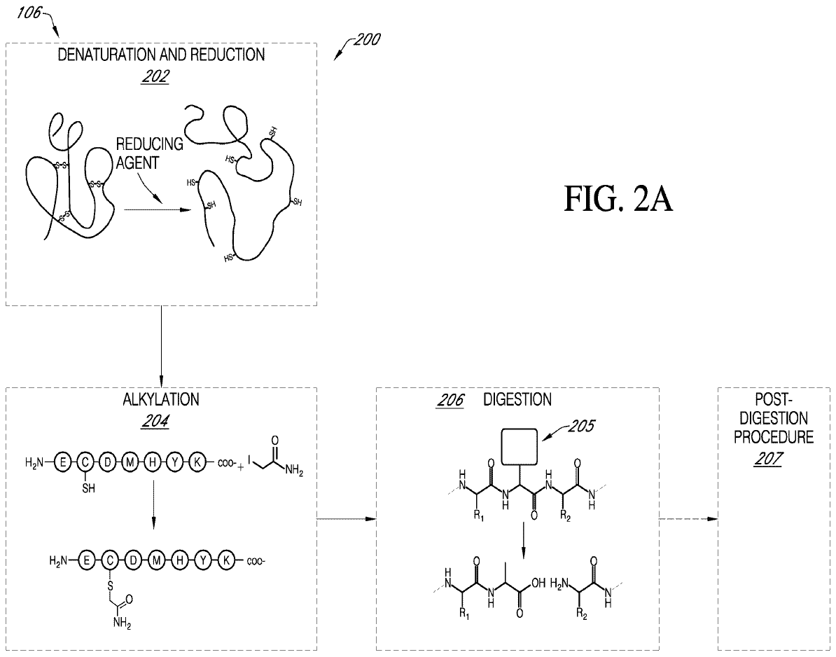

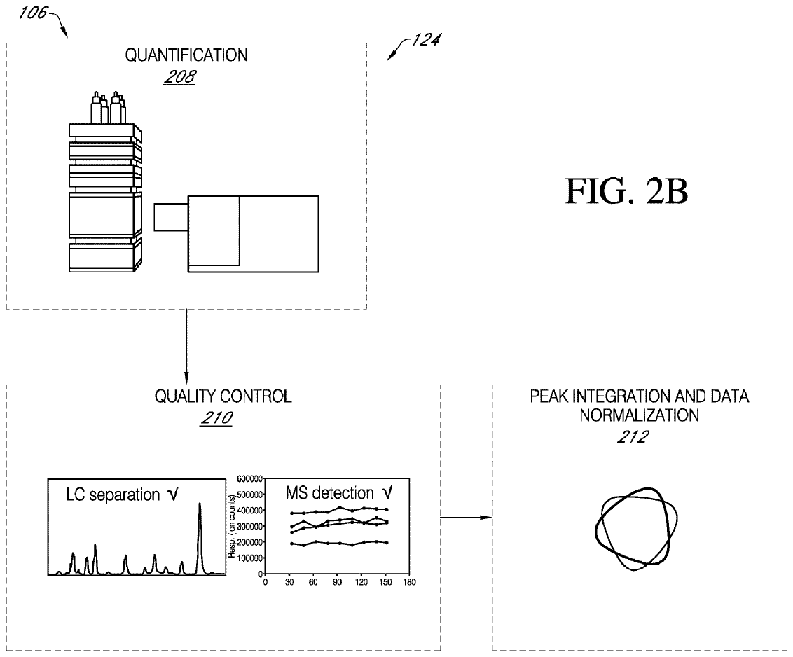

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

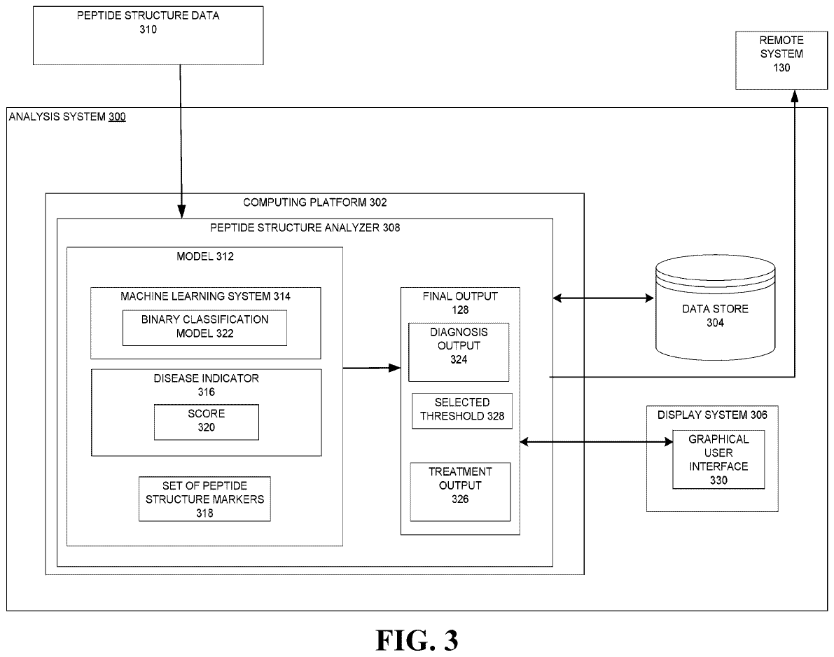

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for a pancreatic cancer (PC) disease state based on set of peptide structures 318 identified as being associated with the PC disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the PC disease state. In various embodiments, disease indicator 316 can include a score 320. Score 320 indicates whether the PC disease state is present or not. For example, score 320 may be, a probability score that indicates how likely it is that the biological sample 112 evidences the presence of the PC disease state.

In some embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the PC disease state based on training of model 312. In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures identified in Table 1 below in Section VI.A, such as with respect to a first group of peptide structures in Group I. In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures identified in Table 8 below in Section IX.B, such as with respect to a second group of peptide structures in Group II. In some cases, the number of peptide structures selected from Table 1 or Table 8 for inclusion in set of peptide structures 318 may be based on, for example, a desired level of accuracy.

In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, or all 31 of the peptide structures PS-1 to PS-8, PS-10 to PS-14, PS-16 to PS-19, PS-21 to PS-25, PS-28 to PS-29, PS-31 to PS-34, and PS-36 to PS-38 in Table 1. In some embodiments, set of peptide structures 318 additionally includes at least 1, at least 2, at least 3, at least 4, at least 5, at least 6, or all 7 of the remaining peptide structures PS-9, PS-15, PS-20, PS-26, PS-27, PS30, and PS-35 in Table 1. In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-1 to PS-22 in Table 8.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In other embodiments, final output 128 includes diagnosis output 324, treatment output 326, or both. Diagnosis output 324 may include, for example, a diagnosis for the PC disease state. The diagnosis can include a positive diagnosis or a negative diagnosis for the PC disease state. In one or more embodiments, generating diagnosis output 324 may include comparing score 320 to selected threshold 328 to determine the diagnosis. Selected threshold 328 may be, for example, without limitation, (e.g., 0.4, 0.5, 0.6, etc.). For example, when selected threshold 328 is set to 0.5, a score 320 above 0.5 may indicate the presence of the PC disease state and be output in diagnosis output 324 as a positive diagnosis. Treatment output 326 may include, for example, at least one of an identification of a treatment for the subject, a treatment plan for administering the treatment, or both. Treatment for pancreatic cancer may include, for example, but is not limited to, at least one of radiation therapy, chemoradiotherapy, surgery, a targeted drug therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 330 in display system 306 for viewing by a human operator

### V.A.2. Computer Implemented System

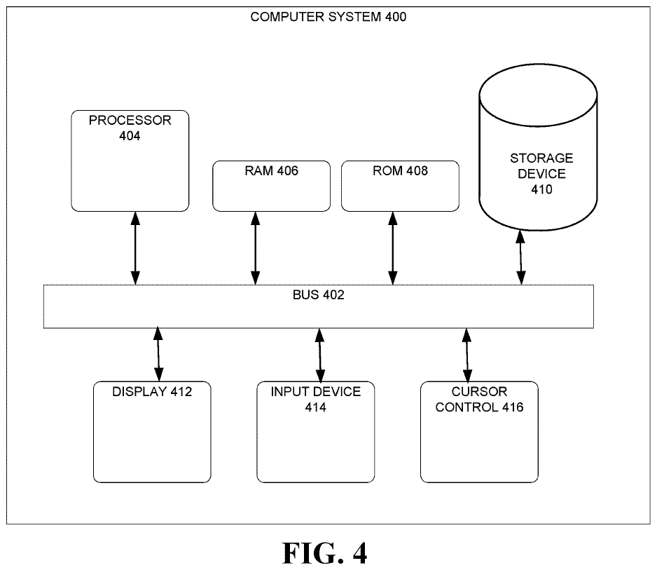

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis Based on Peptide Structure Data Analysis-Group I

### VI.A. General Methodology

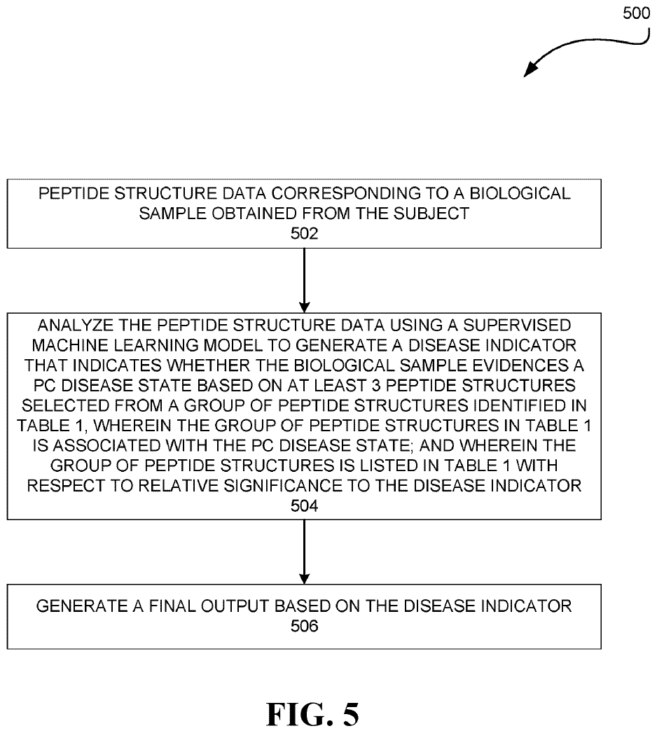

FIG. 5 is a flowchart of a process for diagnosing a subject with respect to a pancreatic cancer (PC) disease state in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject. It should be understood that the same process 500 described in FIG. 5 can be used to generate diagnosis outputs for a subject using different sets of peptide structure data obtained from a subject or subjects, such as that related to Group I set of peptide structure data. That is, process 500 can be implemented by analyzing distinctly different sets of peptide structure data (i.e., different groupings of peptide structures) measured from a subject to generate separate diagnosis outputs for the subject. In various embodiments, process 500 can be applied to a set of peptide structure data provided in Tables 1-7C, as discussed below. In various embodiments, process 500 can be applied to a different set of peptide structure data provided in Tables 8-14, as discussed below.

### VI.B. Process 500 Diagnosis Using Tables 1-7C

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1, with the peptide sequence being one of SEQ ID NOS: 18-40 as defined in Table 1. In various embodiments, other sets of peptides sequences can also be utilized. For example, in some cases at least one peptide structure includes a glycopeptide structure having a peptide sequence and a glycan structure linked to the peptide sequence at a linking site of the peptide sequence, as identified in Table 8, with the peptide sequence being one of SEQ ID NOS: 18, 21, 25, 28, 32, 51-67 as defined in Table 8.

Step 504 includes analyzing the peptide structure data using a supervised machine learning model to generate a disease indicator that indicates whether the biological sample evidences a PC disease state based on at least 3 peptide structures selected from a group of peptide structures identified in Table 1 (below). In step 504, the group of peptide structures in Table 1 is associated with the PC disease state. The group of peptide structures is listed in Table 1 with respect to relative significance to the disease indicator.

In one or more embodiments, the at least 3 peptide structures includes at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, or all 31 of the peptide structures PS-1 to PS-8, PS-10 to PS-14, PS-16 to PS-19, PS-21 to PS-25, PS-28 to PS-29, PS-31 to PS-34, and PS-36 to PS-38 in Table 1. In some embodiments, the at least 3 peptide structures additionally include at least 1, at least 2, at least 3, at least 4, at least 5, at least 6, or all 7 of the remaining peptide structures PS-9, PS-15, PS-20, PS-26, PS-27, PS-30, and PS-35 in Table 1.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is positive for the PC disease state and the supervised machine learning model is configured to generate an output that identifies the biological sample as either evidencing (“positive for”) the PC disease state when the disease indicator is greater than a selected threshold or not evidencing (“negative for”) the PC disease state when the disease indicator is not greater than the selected threshold. The selected threshold may be, for example, 0.30, 0.35, 0.40, 0.45, 0.50, 0.55, or some other threshold. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the PC disease state if the biological sample evidences the PC disease state based on the disease indicator. The diagnosis may be, for example, “negative” if the biological sample does not evidence the PC disease state based on the disease indicator. A negative diagnosis may mean that the biological sample has a non-pancreatic cancer (PC) state (e.g., healthy, control, etc.). The negative diagnosis for the PC disease state can include at least one of a healthy state, a benign pancreatitis state, or a control state.

Generating the diagnosis output in step 506 may include determining that the score falls above a selected threshold and generating a positive diagnosis for the PC disease state. Alternatively, step 506 can include determining that the score falls below a selected threshold and generating a negative diagnosis for the PC disease state. In some scoring systems, the score can include a probability score and the selected threshold can be 0.5. In other scoring systems, the selected threshold can fall within a range between 0.4 and 0.6.

In one or more embodiments, the final output in step 506 may include a treatment output if the diagnosis output indicates a positive diagnosis for the PC disease state. The treatment output may include, for example, at least one of an identification of a treatment for the subject, a treatment plan for administering the treatment, or both. Treatment for pancreatic cancer may include, for example, but is not limited to, at least one of radiation therapy, chemoradiotherapy, surgery, a targeted drug therapy, immunotherapy, chemotherapy, or some other form of treatment. The treatment plan may include, for example, but is not limited to, a timeline or schedule for administering the treatment, dosing information, other treatment-related information, or a combination thereof. Chemotherapy may comprise one or more of Gemcitabine, Nab-paclitaxel, 5-fluorouracil (F-5U), Irinotecan, Oxaliplatin, Capecitabine, Cisplatin, and Liposomal Irinotecan. In specific embodiments, the chemotherapy comprises (1) Gemcitabine plus nab-paclitaxel, and/or (2) 5-FU, irinotecan, and oxaliplatin. In specific cases, the patient is provide up to two dose reductions for nab-paclitaxel (to 100 mg/m² and 75 mg/m²) and gemcitabine (to 800 mg/m² and 600 mg/m²).

TABLE 1
Group I Peptide Structures associated with Pancreatic Cancer

| | | | | | Linking | Linking | |
|---|---|---|---|---|---|---|---|
| | | (Protein) | (Peptide) | Mono- | Site Pos. | Site Pos. | Glycan |
| PS-ID | | SEQ ID | SEQ ID | isotopic | in Protein | in Peptide | Structure |
| NO. | Peptide Structure (PS) NAME | NO. | NO. | mass (Da) | Sequence | Sequence | GL NO. |
| PS-1 | APOA1_DLATVYVDVLK | 1 | 18 | N/A | N/A | N/A | N/A |
| PS-2 | A2MG_55_5402 | 2 | 19 | 4601.00 | 55 | 9 | 5402 |
| PS-3 | HRG_125_5402 | 3 | 20 | 4218.74 | 125 | 5 | 5402 |
| PS-4 | HPT_207_121005 | 4 | 21 | 6888.63 | 207, 211 | 5, 9 | 121005 |
| PS-5 | HPT_207_11904 | 4 | 21 | 6232.40 | 207, 211 | 5, 9 | 11904 |
| PS-6 | AGP1_72MC_6503 | 5 | 22 | 5755.45 | 72MC | 15 | 6503 |
| PS-7 | AGP2_72MC_6503 | 6 | 22 | 5755.45 | 72MC | 15 | 6503 |
| PS-8 | A2MG_869_5402 | 2 | 23 | 5617.39 | 869 | 6 | 5402 |
| PS-9 | A1AT_AVLTIDEK | 7 | 24 | N/A | N/A | N/A | N/A |
| PS-10 | AACT_271_7602 | 8 | 25 | 4686.91 | 271 | 4 | 7602 |
| PS-11 | HPT_241_7613 | 4 | 26 | 5166.19 | 241 | 6 | 7613 |
| PS-12 | HEMO_240_5402 | 9 | 27 | 4055.56 | 240 | 1 | 5402 |
| PS-13 | HEMO_246_5402 | 9 | 27 | 4055.56 | 246 | 1 | 5402 |
| PS-14 | TRFE_432_6503 | 10 | 28 | 4336.74 | 432 | 12 | 6503 |
| PS-15 | IGJ_71_5401 | 11 | 29 | 3141.29 | 71 | 2 | 5401 |
| PS-16 | CFAH_882_5401 | 12 | 30 | 3933.66 | 882 | 15 | 5401 |
| PS-17 | IGA2_205_5410 | 13 | 31 | 2726.19 | 205 | 6 | 5410 |
| PS-18 | IGG1_297_3510 | 13 | 32 | 2836.12 | 297 | 5 | 3510 |
| PS-19 | IGG1_297_3410 | 13 | 32 | 2633.04 | 297 | 5 | 3410 |
| PS-20 | FETUA_156_6503 | 14 | 33 | 4631.84 | 156 | 12 | 6503 |
| PS-21 | IGG1_297_4400 | 13 | 32 | 2649.03 | 297 | 5 | 4400 |
| PS-22 | IGG1_297_4410 | 13 | 32 | 2795.09 | 297 | 5 | 4410 |
| PS-23 | IGG1_297_4410 | 13 | 32 | 2795.09 | 297 | 5 | 4410 |
| PS-24 | IGG1_297_4411 | 13 | 32 | 3086.19 | 297 | 5 | 4411 |
| PS-25 | IGG1_297_4510 | 13 | 32 | 2998.17 | 297 | 5 | 4510 |
| PS-26 | HPT_184_5401 | 4 | 34 | 4592.06 | 184 | 6 | 5401 |
| PS-27 | HPT_207_11915 | 4 | 21 | 6669.56 | 207, 211 | 5, 9 | 11915 |
| PS-28 | IGG1_297_NLFLNHSENATAK | 13 | 32 | 1188.50 | N/A | 5 | N/A |
| PS-29 | IGG1_297_5510 | 13 | 32 | 3160.22 | 297 | 5 | 5510 |
| PS-30 | IGG1_297_5411 | 13 | 32 | 3248.24 | 297 | 5 | 5411 |
| PS-31 | IGG1_297_5410 | 13 | 32 | 2957.14 | 297 | 5 | 5410 |
| PS-32 | IGG1_297_5400 | 13 | 32 | 2811.09 | 297 | 5 | 5400 |
| PS-33 | FETUA_176_6502 | 14 | 35 | 4934.05 | 176 | 11 | 6502 |
| PS-34 | IGM_46_4310 | 15 | 36 | 2687.12 | 46 | 3 | 4310 |
| PS-35 | AGP1_93_7613 | 5 | 37 | 5287.08 | 93 | 7 | 7613 |
| PS-36 | ANT_187_5402 | 16 | 38 | 4381.83 | 187 | 5 | 5402 |
| PS-37 | CO8A_LYYGDDEK | 17 | 39 | N/A | N/A | N/A | N/A |
| PS-38 | AGP1_103_9804 | 5 | 40 | 5022.87 | 103 | 2 | 9804 |

Table 1 includes the Peptide Structure Identification Number (PS-ID NO.) that is a reference number for a particular peptide or glycopeptide. The Peptide Structure Name (PS-Name, e.g., A2MG_55_5402), which is a reference code for the protein name (e.g., A2MG), followed by the glycan linking site position in the protein (e.g., the number 55 that is in between two underscores and represents a sequential amino acid position in protein A2MG), and followed by the glycan structure GL number (e.g., the number 5402 that is preceded by an underscore and represents a glycan composition Hex(5)HexNAc(4)Fuc(0)NeuAc(2). The Protein Sequence ID No of Table 1 corresponds to the corresponding protein name, and Uniprot ID of Table 5. The Peptide Sequence ID No of Table 1 respectively corresponds to the corresponding peptide sequence of Table 4. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 6.

In some instances of the Peptide Structure (PS) NAME, subsequent to the prefix, there is a number noted with the notation MC that indicates that there was a miscleavage at position in the peptide sequence as noted by the number.

VI.C. Training the Model to Diagnose with Respect to the PC Disease State

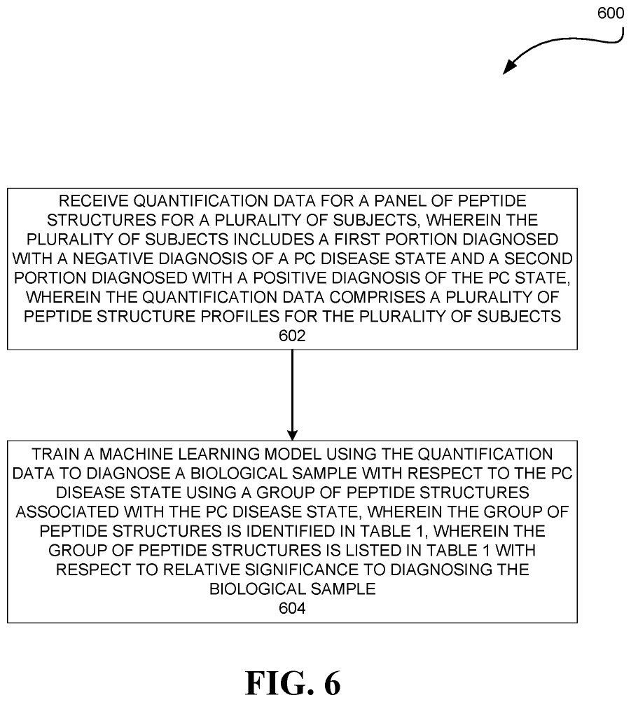

FIG. 6 is a flowchart of a process for training a model to diagnose a subject with respect to a pancreatic cancer (PC) disease state in accordance with one or more embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation for training the model used in the process 500 in FIG. 5.

Step 602 includes receiving quantification data for a panel of peptide structures for a plurality of subjects. The plurality of subjects includes a first portion diagnosed with a negative diagnosis of a PC disease state and a second portion diagnosed with a positive diagnosis of the PC disease state. The quantification data comprises a plurality of peptide structure profiles for the plurality of subjects.

Step 604 includes training a machine learning model using the quantification data to diagnose a biological sample with respect to the PC disease state using a group of peptide structures associated with the PC disease state (e.g., the group of peptide structures is identified in Table 1). The group of peptide structures is listed in Table 1 with respect to relative significance to diagnosing the biological sample. Step 604 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the PC disease state and a negative diagnosis for any subject of the plurality of subjects determined not to have the PC disease state.

The machine learning model can include a binary classification model. Some binary classification models can include logistical regression models. Some logistical regression models can include LASSO regression models.

An alternative or additional step in process 600 can include performing a differential expression analysis using initial training data to compare a first portion of the plurality of subjects diagnosed with the positive diagnosis for the PC disease state versus a second portion of the plurality of subjects diagnosed with the negative diagnosis for the PC disease state.

An alternative or additional step in process 600 can include identifying a training group of peptide structures based on the differential expression analysis for use as prognostic markers for the PC disease state.

An alternative or additional step in process 600 can include forming the training data based on the training group of peptide structures identified.

An alternative or additional step in process 600 can include identifying a training group of peptide structures based on the differential expression analysis, wherein the training group of peptide structures is a subset of the plurality of peptide structures relevant to diagnosing the PC disease state. The subset may be identified based on at least one of fold-changes, false discovery rates, or p-values computed as part of the differential expression analysis.

An alternative or additional step in process 600 can include training a machine learning model, using the quantification data for the training group of peptide structures, to diagnose a subject of a biological sample with respect to the PC disease state using a group of peptide structures associated with the PC disease state. The group of peptide structures may be a subset of the training group of peptide structures and is identified in Table 1. The group of peptide structures is listed in Table 1 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures of Table 2 below, which include at least a portion of the group of peptide structures identified in Table 1. The markers used for training of the LASSO regression model may, in one or more embodiments, additionally include one or more other peptide structure markers.

TABLE 2
Peptide Structures After LASSO Shrinkage

| Model Marker | PS-ID | Peptide Structure | (Protein) | (Peptide) |
|---|---|---|---|---|
| Index | NO. | (PS) NAME | SEQ ID NO. | SEQ ID NO. |
| 1 | PS-1 | APOA1_DLATVYVDVLK | 1 | 18 |
| 2 | PS-2 | A2MG_55_5402 | 2 | 19 |
| 3 | PS-3 | HRG_125_5402 | 3 | 20 |
| 4 | PS-4 | HPT_207_121005 | 4 | 21 |
| 5 | PS-5 | HPT_207_11904 | 4 | 21 |
| 6 | PS-8 | A2MG_869_5402 | 2 | 23 |
| 7 | PS-9 | A1AT_AVLTIDEK | 7 | 24 |
| 8 | PS-12 | HEMO_240_5402 | 9 | 27 |
| | and/or | | | |
| | PS-13 | | | |
| 9 | PS-14 | TRFE_432_6503 | 10 | 28 |
| 10 | PS-15 | IGJ_71_5401 | 11 | 29 |
| 11 | PS-17 | IGA2_205_5410 | 13 | 31 |
| 12 | PS-20 | FETUA_156_6503 | 14 | 33 |
| 13 | PS-26 | HPT_184_5401 | 4 | 34 |
| 14 | PS-33 | FETUA_176_6502 | 14 | 35 |
| 15 | PS-34 | IGM_46_4310 | 15 | 36 |
| 16 | PS-35 | AGP1_93_7613 | 5 | 37 |
| 17 | PS-36 | ANT_187_5402 | 16 | 38 |
| 18 | PS-37 | COSA_LYYGDDEK | 17 | 39 |
| 19 | PS-38 | AGP1_103_9804 | 5 | 40 |

In one or more embodiments, a subset of the markers identified in Table 2 may be used for training of the LASSO regression model. Alternatively, the markers identified in Table 2 may be a subset for training of the LASSO regression model. For example, the LASSO regression model may be trained using at least one other marker in addition to those identified in Table 2. In training the LASSO regression model, any quantification data for peptide structures PS-6 and PS-7 were treated as being for the same marker and thus these two peptide structures were considered as a single marker. Further, any quantification data for peptide structures PS-12 and PS-13 were treated as being for the same marker and thus these two peptide structures were considered as a single marker (Model Marker Index 8).

### VI.D. Monitoring a Subject for a Pancreatic Cancer Disease State

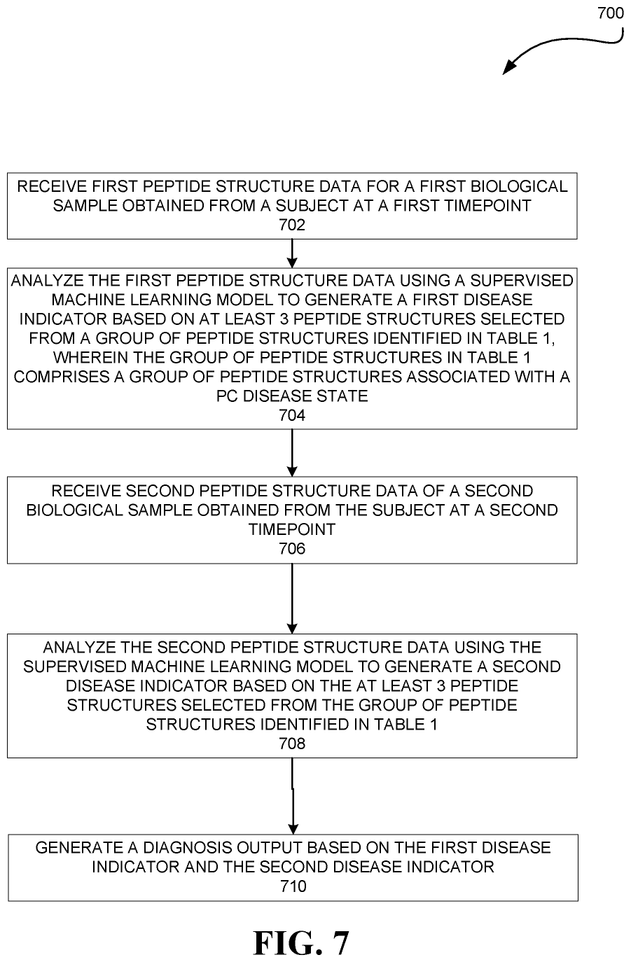

FIG. 7 is a flowchart of a process for monitoring a subject for a pancreatic cancer (PC) disease state in accordance with one or more embodiments. Process 700 may be implemented using, for example, at least a portion of workflow 100 as described in FIGS. 1, 2A, and 2B and/or analysis system 300 as described in FIG. 3.

Step 702 includes receiving first peptide structure data for a first biological sample obtained from a subject at a first timepoint.

Step 704 includes analyzing the first peptide structure data using a supervised machine learning model to generate a first disease indicator based on at least 3 peptide structures selected from a group of peptide structures identified in Table 1. The group of peptide structures in Table 1 includes a group of peptide structures associated with a PC disease state in accordance with various embodiments. The supervised machine can be a binary classification model. In some embodiments, the binary classification model can be a logistical regression model.

Step 706 includes receiving second peptide structure data of a second biological sample obtained from the subject at a second timepoint.

Step 708 includes analyzing the second peptide structure data using the supervised machine learning model to generate a second disease indicator based on the at least 3 peptide structures selected from the group of peptide structures identified in Table 1.

Step 710 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the PC disease state and the second biological sample evidences the positive diagnosis for the PC disease. In other embodiments, the diagnosis output identifies whether a non-PC disease state has progressed to the PC disease state, wherein the non-PC disease state includes either a healthy state or a benign pancreatitis state.

### VII. Group I Peptide Structure and Product Ion Compositions, Kits and Reagents

Aspects of the disclosure include compositions comprising one or more of the peptide structures listed in Table 1. In some embodiments, a composition comprises a plurality of the peptide structures listed in Table 1. In some embodiments, a composition comprises 1, 2, 3, 4, 5, 6, 7, 8, 9, 10, 11, 12, 13, 14, 15, 16, 17, 18, or 19 of the peptide structures listed in Table 1. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 18-40, listed in Table 1.

Aspects of the disclosure include compositions comprising one or more precursor ions having a defined charge and/or defined mass-to-charge (m/z) ratio, as listed in Table 3. Aspects of the disclosure include compositions comprising one or more product ions having a defined mass-to-charge (m/z) ratio, which product ions are produced by converting a peptide structure described herein (e.g., a peptide structure listed in Table 1) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 1). In some embodiments, a composition comprises a set of the product ions listed in Table 3, having an m/z ratio selected from the list provided for each peptide structure in Table 1.

In some embodiments, a composition comprises at least one of peptide structures PS-1 to PS-38 identified in Table 1. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, or all 31 of the peptide structures PS-1 to PS-8, PS-10 to PS-14, PS-16 to PS-19, PS-21 to PS-25, PS-28 to PS-29, PS-31 to PS-34, and PS-36 to PS-38 in Table 1. In some embodiments, the at least 3 peptide structures additionally include at least 1, at least 2, at least 3, at least 4, at least 5, at least 6, or all 7 of the remaining peptide structures PS-9, PS-15, PS-20, PS-26, PS-27, PS30, and PS-35 in Table 1.

In some embodiments, a composition comprises a peptide structure or a product ion. In some embodiments, the peptide structure or product ion comprises an amino acid sequence having at least 90% sequence identity to any one of SEQ ID NOS: 18-40, as identified in Table 4, corresponding to peptide structures PS-1 to PS-38 in Table 1.

In some embodiments, a composition comprises a peptide structure or a product ion. In some embodiments, the peptide structure or product ion comprises an amino acid sequence having at least 90% sequence identity to any one of SEQ ID NOS: 18-23, 25-28, 30-32, 35-36, and 38-40, as identified in Table 4, corresponding to peptide structures PS-1 to PS-8, PS-10 to PS-14, PS-16 to PS-19, PS-21 to PS-25, PS-28 to PS-29, PS-31 to PS-34, and PS-36 to PS-38 in Table 1.

In some embodiments, the product ion is selected as one from a group consisting of product ions identified in Table 3, including product ions falling within an identified m/z range of the m/z ratio identified in Table 3 and characterized as having a precursor ion having an m/z ratio within an identified m/z range of the m/z ratio identified in Table 3. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 3, and characterized as having a precursor ion having an m/z ratio that falls within at least one of first range (±0.5), a second range (±1.0), or a third range (±1.0 of the precursor ion m/z ratio identified in Table 3.

Table 3 shows various parameters associated with the identification of the peptide and glycopeptides using LC and MRM-MS. The retention time (RT) represents the amount of time in minutes for the peptide to elute from the chromatography column. The collision energy represents the energy applied to the peptide for creating fragments (i.e., product ions) such as, for example, in the 2nd quadrupole of the triple quadrupole MS. The first precursor m/z represents a ratio value associated with an ionized form having a precursor charge for the peptide or glycopeptide. The precursor ion is associated with a first product ion having a m/z ratio that was formed from a collision and the second precursor ion is associated with a second product ion having a m/z ratio that was formed from a collision.

TABLE 3
Mass Spectrometry-Related Characteristics for the Peptide
Structures associated with Pancreatic Cancer

| PS-ID | RT | Collision | Precursor | Precursor | Product |
|---|---|---|---|---|---|
| NO. | (min) | Energy | m/z | Charge | m/z |
| PS-1 | 35.2 | 17 | 618.3 | 2 | 736.4 |
| PS-2 | 42.1 | 25 | 1151.7 | 4 | 366.1 |
| PS-3 | 28.4 | 25 | 1056.2 | 4 | 366.1 |
| PS-4 | 13.3 | 35 | 1378.9 | 5 | 366.1 |
| PS-5 | 13.2 | 31 | 1247.7 | 5 | 366.1 |
| PS-6 | 41 | 28 | 1152.7 | 5 | 366.1 |
| PS-7 | 41 | 28 | 1152.7 | 5 | 366.1 |
| PS-8 | 35.9 | 25 | 1124.9 | 5 | 366.1 |
| PS-9 | 15.1 | 11 | 444.8 | 2 | 718.4 |
| PS-10 | 30.2 | 28 | 1173.2 | 4 | 366.1 |
| PS-11 | 30.7 | 32 | 1292.8 | 4 | 366.1 |
| PS-12 | 7.3 | 30 | 1015.2 | 4 | 366.1 |
| PS-13 | 7.3 | 30 | 1015.2 | 4 | 366.1 |
| PS-14 | 27.4 | 27 | 1085.4 | 4 | 366.1 |
| PS-15 | 15.3 | 26 | 1048.1 | 3 | 366.1 |
| PS-16 | 14.8 | 25 | 984.7 | 4 | 366.1 |
| PS-17 | 12.2 | 22 | 909.8 | 3 | 366.1 |
| PS-18 | 8.1 | 15 | 946.5 | 3 | 204.1 |
| PS-19 | 7.9 | 21 | 879 | 3 | 204.1 |
| PS-20 | 27.7 | 29 | 1159.5 | 4 | 366.1 |
| PS-21 | 7.9 | 21 | 884.4 | 3 | 204.1 |
| PS-22 | 7.8 | 22 | 932.8 | 3 | 204.1 |
| PS-23 | 7.8 | 15 | 699.8 | 4 | 204.1 |
| PS-24 | 8.3 | 35 | 1029.8 | 3 | 204.1 |
| PS-25 | 8 | 15 | 1000.7 | 3 | 204.1 |
| PS-26 | 32.4 | 28 | 1149.4 | 4 | 366.1 |
| PS-27 | 13.4 | 34 | 1335.1 | 5 | 366.1 |
| PS-28 | 8.3 | 13 | 595.3 | 2 | 640.3 |
| PS-29 | 8 | 20 | 1054.7 | 3 | 366.1 |
| PS-30 | 8.2 | 27 | 1084.1 | 3 | 366.1 |
| PS-31 | 7.8 | 24 | 987.1 | 3 | 366.1 |
| PS-32 | 7.8 | 22 | 938.4 | 3 | 366.1 |
| PS-33 | 30.2 | 31 | 1234.3 | 4 | 366.1 |
| PS-34 | 6.3 | 30 | 896.7 | 3 | 204.1 |
| PS-35 | 23.1 | 33 | 1323.1 | 4 | 366.1 |
| PS-36 | 40.9 | 25 | 1097 | 4 | 366.1 |
| PS-37 | 10.3 | 13 | 501.7 | 2 | 726.3 |
| PS-38 | 5.6 | 25 | 1256.8 | 4 | 366.1 |

Table 4 defines the peptide sequences for SEQ ID NOS: 18-40 from Table 1. Table 4 further identifies a corresponding protein SEQ ID NO. for each peptide sequence.

TABLE 4
Peptide SEQ ID NOS

| | | Corresponding |
|---|---|---|
| SEQ ID | | Protein |
| NO: | Peptide Sequence | SEQ ID NO: |
| 18 | DLATVYVDVLK | 1 |
| 19 | GCVLLSYLNETVTVSASLESVR | 2 |
| 20 | VIDFNCTTSSVSSALANTK | 3 |
| 21 | NLFLNHSENATAK | 4 |
| 22 | SVQEIQATFFYFTPNKTEDTIFLR | 5, 6 |
| 23 | SLGNVNFTVSAEALESQELCGTEV | 2 |
| | PSVPEHGR | |
| 24 | AVLTIDEK | 7 |
| 25 | YTGNASALFILPDQDK | 8 |
| 26 | VVLHPNYSQVDIGLIK | 4 |
| 27 | NGTGHGNSTHHGPEYMR | 9 |
| 28 | CGLVPVLAENYNK | 10 |
| 29 | ENISDPTSPLR | 11 |
| 30 | IPCSQPPQIEHGTINSSR | 12 |
| 31 | TPLTANITK | 13 |
| 32 | EEQYNSTYR | 13 |
| 33 | VCQDCPLLAPLNDTR | 14 |
| 34 | MVSHHNLTTGATLINEQWLLTTAK | 4 |
| 35 | AALAAFNAQNNGSNFQLEEISR | 14 |
| 36 | YKNNSDISSTR | 15 |
| 37 | QDQCIYNTTYLNVQR | 5 |
| 38 | SLTFNETYQDISELVYGAK | 16 |
| 39 | LYYGDDEK | 17 |
| 40 | ENGTISR | 5 |

Table 5 identifies the proteins of SEQ ID NOS: 1-17 from Table 1. Table 5 identifies a corresponding protein abbreviation and protein name for each of protein SEQ ID NOS: 1-17. Further, Table 5 identifies a corresponding Uniprot ID for each of protein SEQ ID NOS: 1-17.

TABLE 5
Protein SEQ ID NOS

| SEQ | Prot | | Uniprot | |
|---|---|---|---|---|
| ID NO. | Abbrev. | Protein Name | ID | Protein Sequence |
| 1 | APOA1 | Apolipoprotein A-I | P02647 | MKAAVLTLAVLFLTGSQARHFWQQDEPPQSPW |
| | | | | DRVKDLATVYVDVLKDSGRDYVSQFEGSALG |
| | | | | KQLNLKLLDNWDSVTSTFSKLREQLGPVTQEF |
| | | | | WDNLEKETEGLRQEMSKDLEEVKAKVQPYLD |
| | | | | DFQKKWQEEMELYRQKVEPLRAELQEGARQK |
| | | | | LHELQEKLSPLGEEMRDRARAHVDALRTHLAP |
| | | | | YSDELRQRLAARLEALKENGGARLAEYHAKAT |
| | | | | EHLSTSEKAKPALEDLRQGLLPVLESFKVSFLSA |
| | | | | LEEYTKKLNTQ |
| 2 | A2MG | Alpha-2-macroglobulin | P01023 | MGKNKLLHPSLVLLLLVLLPTDASVSGKPQYM |
| | | | | VLVPSLLHTETTEKGCVLLSYLNETVTVSASLES |
| | | | | VRGNRSLFTDLEAENDVLHCVAFAVPKSSSNEE |
| | | | | VMFLTVQVKGPTQEFKKRTTVMVKNEDSLVFV |
| | | | | QTDKSIYKPGQTVKFRVVSMDENFHPLNELIPL |
| | | | | VYIQDPKGNRIAQWQSFQLEGGLKQFSFPLSSEP |
| | | | | FQGSYKVVVQKKSGGRTEHPFTVEEFVLPKFEV |
| | | | | QVTVPKIITILEEEMNVSVCGLYTYGKPVPGHV |
| | | | | TVSICRKYSDASDCHGEDSQAFCEKFSGQLNSH |
| | | | | GCFYQQVKTKVFQLKRKEYEMKLHTEAQIQEE |
| | | | | GTVVELTGRQSSEITRTITKLSFVKVDSHFRQGI |
| | | | | PFFGQVRLVDGKGVPIPNKVIFIRGNEANYYSN |
| | | | | ATTDEHGLVQFSINTTNVMGTSLTVRVNYKDR |
| | | | | SPCYGYQWVSEEHEEAHHTAYLVFSPSKSFVHL |
| | | | | EPMSHELPCGHTQTVQAHYILNGGTLLGLKKLS |
| | | | | FYYLIMAKGGIVRTGTHGLLVKQEDMKGHFSIS |
| | | | | IPVKSDIAPVARLLIYAVLPTGDVIGDSAKYDVE |
| | | | | NCLANKVDLSFSPSQSLPASHAHLRVTAAPQSV |
| | | | | CALRAVDQSVLLMKPDAELSASSVYNLLPEKD |
| | | | | LTGFPGPLNDQDNEDCINRHNVYINGITYTPVSS |
| | | | | TNEKDMYSFLEDMGLKAFTNSKIRKPKMCPQL |
| | | | | QQYEMHGPEGLRVGFYESDVMGRGHARLVHV |
| | | | | EEPHTETVRKYFPETWIWDLVVVNSAGVAEVG |
| | | | | VTVPDTITEWKAGAFCLSEDAGLGISSTASLRAF |
| | | | | QPFFVELTMPYSVIRGEAFTLKATVLNYLPKCIR |
| | | | | VSVQLEASPAFLAVPVEKEQAPHCICANGRQTV |
| | | | | SWAVTPKSLGNVNFTVSAEALESQELCGTEVPS |
| | | | | VPEHGRKDTVIKPLLVEPEGLEKETTFNSLLCPS |
| | | | | GGEVSEELSLKLPPNVVEESARASVSVLGDILGS |
| | | | | AMQNTQNLLQMPYGCGEQNMVLFAPNIYVLD |
| | | | | YLNETQQLTPEIKSKAIGYLNTGYQRQLNYKHY |
| | | | | DGSYSTFGERYGRNQGNTWLTAFVLKTFAQAR |
| | | | | AYIFIDEAHITQALIWLSQRQKDNGCFRSSGSLL |
| | | | | NNAIKGGVEDEVTLSAYITIALLEIPLTVTHPVV |
| | | | | RNALFCLESAWKTAQEGDHGSHVYTKALLAY |
| | | | | AFALAGNQDKRKEVLKSLNEEAVKKDNSVHW |
| | | | | ERPQKPKAPVGHFYEPQAPSAEVEMTSYVLLA |
| | | | | YLTAQPAPTSEDLTSATNIVKWITKQQNAQGGF |
| | | | | SSTQDTVVALHALSKYGAATFTRTGKAAQVTI |
| | | | | QSSGTFSSKFQVDNNNRLLLQQVSLPELPGEYS |
| | | | | MKVTGEGCVYLQTSLKYNILPEKEEFPFALGVQ |
| | | | | TLPQTCDEPKAHTSFQISLSVSYTGSRSASNMAI |
| | | | | VDVKMVSGFIPLKPTVKMLERSNHVSRTEVSSN |
| | | | | HVLIYLDKVSNQTLSLFFTVLQDVPVRDLKPAI |
| | | | | VKVYDYYETDEFAIAEYNAPCSKDLGNA |
| 3 | HRG | Histidine-rich | P04196 | MKALIAALLLITLQYSCAVSPTDCSAVEPEAEK |
| | | Glycoprotein | | ALDLINKRRRDGYLFQLLRIADAHLDRVENTTV |
| | | | | YYLVLDVQESDCSVLSRKYWNDCEPPDSRRPS |
| | | | | EIVIGQCKVIATRHSHESQDLRVIDENCTTSSVSS |
| | | | | ALANTKDSPVLIDFFEDTERYRKQANKALEKY |
| | | | | KEENDDFASFRVDRIERVARVRGGEGTGYFVD |
| | | | | FSVRNCPRHHFPRHPNVFGFCRADLFYDVEALD |
| | | | | LESPKNLVINCEVFDPQEHENINGVPPHLGHPFH |
| | | | | WGGHERSSTTKPPFKPHGSRDHHHPHKPHEHG |
| | | | | PPPPPDERDHSHGPPLPQGPPPLLPMSCSSCQHA |
| | | | | TFGTNGAQRHSHNNNSSDLHPHKHHSHEQHPH |
| | | | | GHHPHAHHPHEHDTHRQHPHGHHPHGHHPHG |
| | | | | HHPHGHHPHGHHPHCHDFQDYGPCDPPPHNQG |
| | | | | HCCHGHGPPPGHLRRRGPGKGPRPFHCRQIGSV |
| | | | | YRLPPLRKGEVLPLPEANFPSFPLPHHKHPLKPD |
| | | | | NQPFPQSVSESCPGKFKSGFPQVSMFFTHTFPK |
| 4 | HPT | Haptoglobin | P00738 | MSALGAVIALLLWGOLFAVDSGNDVTDIADDG |
| | | | | CPKPPEIAHGYVEHSVRYQCKNYYKLRTEGDG |
| | | | | VYTLNDKKQWINKAVGDKLPECEADDGCPKPP |
| | | | | EIAHGYVEHSVRYQCKNYYKLRTEGDGVYTLN |
| | | | | NEKQWINKAVGDKLPECEAVCGKPKNPANPVQ |
| | | | | RILGGHLDAKGSFPWQAKMVSHHNLTTGATLI |
| | | | | NEQWLLTTAKNLFLNHSENATAKDIAPTLTLYV |
| | | | | GKKQLVEIEKVVLHPNYSQVDIGLIKLKQKVSV |
| | | | | NERVMPICLPSKDYAEVGRVGYVSGWGRNANF |
| | | | | KFTDHLKYVMLPVADQDQCIRHYEGSTVPEKK |
| | | | | TPKSPVGVQPILNEHTFCAGMSKYQEDTCYGD |
| | | | | AGSAFAVHDLEEDTWYATGILSFDKSCAVAEY |
| | | | | GVYVKVTSIQDWVOKTIAEN |
| 5 | AGP1 | Alpha-1-acid | P02763 | MALSWVLTVLSLLPLLEAQIPLCANLVPVPITN |
| | | glycoprotein 1 | | ATLDRITGKWFYIASAFRNEEYNKSVQEIQATFF |
| | | | | YFTPNKTEDTIFLREYQTRQDQCIYNTTYLNVQ |
| | | | | RENGTISRYVGGQEHFAHLLILRDTKTYMLAFD |
| | | | | VNDEKNWGLSVYADKPETTKEQLGEFYEALDC |
| | | | | LRIPKSDVVYTDWKKDKCEPLEKQHEKERKQE |
| | | | | EGES |
| 6 | AGP2 | Alpha-1-acid | P19652 | MALSWVLTVLSLLPLLEAQIPLCANLVPVPITN |
| | | glycoprotein 2 | | ATLDRITGKWFYIASAFRNEEYNKSVQEIQATFF |
| | | | | YFTPNKTEDTIFLREYQTRQNQCFYNSSYLNVQ |
| | | | | RENGTVSRYEGGREHVAHLLFLRDTKTLMFGS |
| | | | | YLDDEKNWGLSFYADKPETTKEQLGEFYEALD |
| | | | | CLCIPRSDVMYTDWKKDKCEPLEKQHEKERKQ |
| | | | | EEGES |
| 7 | A1AT | Alpha-1-antitrypsin | P01009 | MPSSVSWGILLLAGLCCLVPVSLAEDPQGDAA |
| | | | | QKTDTSHHDQDHPTFNKITPNLAEFAFSLYRQL |
| | | | | AHQSNSTNIFFSPVSIATAFAMLSLGTKADTHDE |
| | | | | ILEGLNFNLTEIPEAQIHEGFQELLRTLNQPDSQL |
| | | | | QLTTGNGLFLSEGLKLVDKFLEDVKKLYHSEAF |
| | | | | TVNFGDTEEAKKQINDYVEKGTQGKIVDLVKE |
| | | | | LDRDTVFALVNYIFFKGKWERPFEVKDTEEEDF |
| | | | | HVDQVTTVKVPMMKRLGMFNIQHCKKLSSWV |
| | | | | LLMKYLGNATAIFFLPDEGKLQHLENELTHDIIT |
| | | | | KFLENEDRRSASLHLPKLSITGTYDLKSVLGQL |
| | | | | GITKVFSNGADLSGVTEEAPLKLSKAVHKAVLT |
| | | | | IDEKGTEAAGAMFLEAIPMSIPPEVKFNKPFVFL |
| | | | | MIEQNTKSPLFMGKVVNPTQK |
| 8 | AACT | Alpha-1- | P01011 | MERMLPLLALGLLAAGFCPAVLCHPNSPLDEE |
| | | antichymotrypsin | | NLTQENQDRGTHVDLGLASANVDFAFSLYKQL |
| | | | | VLKAPDKNVIFSPLSISTALAFLSLGAHNTTLTEI |
| | | | | LKGLKFNLTETSEAEIHQSFQHLLRTLNQSSDEL |
| | | | | QLSMGNAMFVKEQLSLLDRFTEDAKRLYGSEA |
| | | | | FATDFQDSAAAKKLINDYVKNGTRGKITDLIKD |
| | | | | LDSQTMMVLVNYIFFKAKWEMPFDPQDTHQSR |
| | | | | FYLSKKKWVMVPMMSLHHLTIPYFRDEELSCT |
| | | | | VVELKYTGNASALFILPDQDKMEEVEAMLLPE |
| | | | | TLKRWRDSLEFREIGELYLPKFSISRDYNLNDIL |
| | | | | LOLGIEEAFTSKADLSGITGARNLAVSQVVHKA |
| | | | | VLDVFEEGTEASAATAVKITLLSALVETRTIVRF |
| | | | | NRPFLMIIVPTDTQNIFFMSKVTNPKQA |
| 9 | HEMO | Hemopexin | P02790 | MARVLGAPVALGLWSLCWSLAIATPLPPTSAH |
| | | | | GNVAEGETKPDPDVTERCSDGWSFDATTLDDN |
| | | | | GTMLFFKGEFVWKSHKWDRELISERWKNFPSP |
| | | | | VDAAFRQGHNSVFLIKGDKVWVYPPEKKEKGY |
| | | | | PKLLQDEFPGIPSPLDAAVECHRGECQAEGVLF |
| | | | | FQGDREWFWDLATGTMKERSWPAVGNCSSAL |
| | | | | RWLGRYYCFQGNQFLRFDPVRGEVPPRYPRDV |
| | | | | RDYFMPCPGRGHGHRNGTGHGNSTHHGPEYM |
| | | | | RCSPHLVLSALTSDNHGATYAFSGTHYWRLDT |
| | | | | SRDGWHSWPIAHQWPQGPSAVDAAFSWEEKL |
| | | | | YLVQGTQVYVFLTKGGYTLVSGYPKRLEKEVG |
| | | | | TPHGIILDSVDAAFICPGSSRLHIMAGRRLWWL |
| | | | | DLKSGAQATWTELPWPHEKVDGALCMEKSLG |
| | | | | PNSCSANGPGLYLIHGPNLYCYSDVEKLNAAK |
| | | | | ALPQPQNVTSLLGCTH |
| 10 | TRFE | Serotransferrin | P02787 | MRLAVGALLVCAVLGLCLAVPDKTVRWCAVS |
| | | | | EHEATKCQSFRDHMKSVIPSDGPSVACVKKAS |
| | | | | YLDCIRAIAANEADAVTLDAGLVYDAYLAPNN |
| | | | | LKPVVAEFYGSKEDPQTFYYAVAVVKKDSGFQ |
| | | | | MNQLRGKKSCHTGLGRSAGWNIPIGLLYCDLP |
| | | | | EPRKPLEKAVANFFSGSCAPCADGTDFPQLCQL |
| | | | | CPGCGCSTLNQYFGYSGAFKCLKDGAGDVAFV |
| | | | | KHSTIFENLANKADRDQYELLCLDNTRKPVDE |
| | | | | YKDCHLAQVPSHTVVARSMGGKEDLIWELLNQ |
| | | | | AQEHFGKDKSKEFQLFSSPHGKDLLFKDSAHGF |
| | | | | LKVPPRMDAKMYLGYEYVTAIRNLREGTCPEA |
| | | | | PTDECKPVKWCALSHHERLKCDEWSVNSVGKI |
| | | | | ECVSAETTEDCIAKIMNGEADAMSLDGGFVYIA |
| | | | | GKCGLVPVLAENYNKSDNCEDTPEAGYFAIAV |
| | | | | VKKSASDLTWDNLKGKKSCHTAVGRTAGWNI |
| | | | | PMGLLYNKINHCRFDEFFSEGCAPGSKKDSSLC |
| | | | | KLCMGSGLNLCEPNNKEGYYGYTGAFRCLVEK |
| | | | | GDVAFVKHQTVPQNTGGKNPDPWAKNLNEKD |
| | | | | YELLCLDGTRKPVEEYANCHLARAPNHAVVTR |
| | | | | KDKEACVHKILRQQQHLFGSNVTDCSGNFCLF |
| | | | | RSETKDLLFRDDTVCLAKLHDRNTYEKYLGEE |
| | | | | YVKAVGNLRKCSTSSLLEACTFRRP |
| 11 | IGJ | Immunoglobulin J chain | P01591 | MKNHLLFWGVLAVFIKAVHVKAQEDERIVLVD |
| | | | | NKCKCARITSRIIRSSEDPNEDIVERNIRIIVPLNN |
| | | | | RENISDPTSPLRTRFVYHLSDLCKKCDPTEVELD |
| | | | | NQIVTATQSNICDEDSATETCYTYDRNKCYTAV |
| | | | | VPLVYGGETKMVETALTPDACYPD |
| 12 | CFAH | Complement Factor H | P08603 | MRLLAKIICLMLWAICVAEDCNELPPRRNTEILT |
| | | | | GSWSDQTYPEGTQAIYKCRPGYRSLGNVIMVC |
| | | | | RKGEWVALNPLRKCQKRPCGHPGDTPFGTFTL |
| | | | | TGGNVFEYGVKAVYTCNEGYQLLGEINYRECD |
| | | | | TDGWTNDIPICEVVKCLPVTAPENGKIVSSAME |
| | | | | PDREYHFGQAVRFVCNSGYKIEGDEEMHCSDD |
| | | | | GFWSKEKPKCVEISCKSPDVINGSPISQKIIYKEN |
| | | | | ERFQYKCNMGYEYSERGDAVCTESGWRPLPSC |
| | | | | EEKSCDNPYIPNGDYSPLRIKHRTGDEITYQCRN |
| | | | | GFYPATRGNTAKCTSTGWIPAPRCTLKPCDYPD |
| | | | | IKHGGLYHENMRRPYFPVAVGKYYSYYCDEHF |
| | | | | ETPSGSYWDHIHCTQDGWSPAVPCLRKCYFPY |
| | | | | LENGYNQNYGRKFVQGKSIDVACHPGYALPKA |
| | | | | QTTVTCMENGWSPTPRCIRVKTCSKSSIDIENGF |
| | | | | ISESQYTYALKEKAKYQCKLGYVTADGETSGSI |
| | | | | TCGKDGWSAQPTCIKSCDIPVFMNARTKNDFT |
| | | | | WFKLNDTLDYECHDGYESNTGSTTGSIVCGYN |
| | | | | GWSDLPICYERECELPKIDVHLVPDRKKDQYKV |
| | | | | GEVLKFSCKPGFTIVGPNSVQCYHFGLSPDLPIC |
| | | | | KEQVQSCGPPPELLNGNVKEKTKEEYGHSEVV |
| | | | | EYYCNPRFLMKGPNKIQCVDGEWTTLPVCIVEE |
| | | | | STCGDIPELEHGWAQLSSPPYYYGDSVEFNCSE |
| | | | | SFTMIGHRSITCIHGVWTQLPQCVAIDKLKKCK |
| | | | | SSNLIILEEHLKNKKEFDHNSNIRYRCRGKEGWI |
| | | | | HTVCINGRWDPEVNCSMAQIQLCPPPPQIPNSH |
| | | | | NMTTTLNYRDGEKVSVLCQENYLIQEGEEITCK |
| | | | | DGRWQSIPLCVEKIPCSQPPQIEHGTINSSRSSQE |
| | | | | SYAHGTKLSYTCEGGFRISEENETTCYMGKWSS |
| | | | | PPQCEGLPCKSPPEISHGVVAHMSDSYQYGEEV |
| | | | | TYKCFEGFGIDGPAIAKCLGEKWSHPPSCIKTDC |
| | | | | LSLPSFENAIPMGEKKDVYKAGEQVTYTCATY |
| | | | | YKMDGASNVTCINSRWTGRPTCRDTSCVNPPT |
| | | | | VQNAYIVSRQMSKYPSGERVRYQCRSPYEMFG |
| | | | | DEEVMCLNGNWTEPPQCKDSTGKCGPPPPIDN |
| | | | | GDITSFPLSVYAPASSVEYQCONLYQLEGNKRIT |
| | | | | CRNGQWSEPPKCLHPCVISREIMENYNIALRWT |
| | | | | AKQKLYSRTGESVEFVCKRGYRLSSRSHTLRTT |
| | | | | CWDGKLEYPTCAKR |
| 13 | IGA2 | Immunoglobulin heavy | P01877 | ASPTSPKVFPLSLDSTPQDGNVVVACLVQGFFP |
| | | constant alpha 2 | | QEPLSVTWSESGQNVTARNFPPSQDASGDLYTT |
| | | | | SSQLTLPATQCPDGKSVTCHVKHYTNSSQDVT |
| | | | | VPCRVPPPPPCCHPRLSLHRPALEDLLLGSEANL |
| | | | | TCTLTGLRDASGATFTWTPSSGKSAVQGPPERD |
| | | | | LCGCYSVSSVLPGCAQPWNHGETFTCTAAHPE |
| | | | | LKTPLTANITKSGNTFRPEVHLLPPPSEELALNE |
| | | | | LVTLTCLARGFSPKDVLVRWLQGSQELPREKY |
| | | | | LTWASRQEPSQGTTTYAVTSILRVAAEDWKKG |
| | | | | ETFSCMVGHEALPLAFTQKTIDRMAGKPTHINV |
| | | | | SVVMAEADGTCY |
| 14 | FETUA | Alpha-2-HS- | P02765 | MKSLVLLLCLAQLWGCHSAPHGPGLIYRQPNC |
| | | glycoprotein | | DDPETEEAALVAIDYINQNLPWGYKHTLNQIDE |
| | | | | VKVWPQQPSGELFEIEIDTLETTCHVLDPTPVAR |
| | | | | CSVRQLKEHAVEGDCDFQLLKLDGKFSVVYAK |
| | | | | CDSSPDSAEDVRKVCQDCPLLAPLNDTRVVHA |
| | | | | AKAALAAFNAQNNGSNFQLEEISRAQLVPLPPS |
| | | | | TYVEFTVSGTDCVAKEATEAAKCNLLAEKQYG |
| | | | | FCKATLSEKLGGAEVAVTCMVFQTQPVSSQPQ |
| | | | | PEGANEAVPTPVVDPDAPPSPPLGAPGLPPAGSP |
| | | | | PDSHVLLAAPPGHQLHRAHYDLRHTFMGVVSL |
| | | | | GSPSGEVSHPRKTRTVVQPSVGAAAGPVVPPCP |
| | | | | GRIRHFKV |
| 15 | IGM | Immunoglobulin heavy | P01871 | GSASAPTLFPLVSCENSPSDTSSVAVGCLAQDFL |
| | | constant mu | | PDSITFSWKYKNNSDISSTRGFPSVLRGGKYAA |
| | | | | TSQVLLPSKDVMQGTDEHVVCKVQHPNGNKE |
| | | | | KNVPLPVIAELPPKVSVFVPPRDGFFGNPRKSKL |
| | | | | ICQATGFSPRQIQVSWLREGKQVGSGVTTDQVQ |
| | | | | AEAKESGPTTYKVTSTLTIKESDWLGQSMFTCR |
| | | | | VDHRGLTFQQNASSMCVPDQDTAIRVFAIPPSF |
| | | | | ASIFLTKSTKLTCLVTDLTTYDSVTISWTRONGE |
| | | | | AVKTHTNISESHPNATFSAVGEASICEDDWNSG |
| | | | | ERFTCTVTHTDLPSPLKQTISRPKGVALHRPDV |
| | | | | YLLPPAREQLNLRESATITCLVTGFSPADVFVQ |
| | | | | WMQRGQPLSPEKYVTSAPMPEPQAPGRYFAHS |
| | | | | ILTVSEEEWNTGETYTCVVAHEALPNRVTERTV |
| | | | | DKSTGKPTLYNVSLVMSDTAGTCY |
| 16 | ANT | Antithrombin-III | P01008 | MYSNVIGTVTSGKRKVYLLSLLLIGFWDCVTCH |
| | | | | GSPVDICTAKPRDIPMNPMCIYRSPEKKATEDE |
| | | | | GSEQKIPEATNRRVWELSKANSRFATTFYQHLA |
| | | | | DSKNDNDNIFLSPLSISTAFAMTKLGACNDTLQ |
| | | | | QLMEVFKFDTISEKTSDQIHFFFAKLNCRLYRK |
| | | | | ANKSSKLVSANRLFGDKSLTFNETYQDISELVY |
| | | | | GAKLQPLDFKENAEQSRAAINKWVSNKTEGRIT |
| | | | | DVIPSEAINELTVLVLVNTIYFKGLWKSKFSPEN |
| | | | | TRKELFYKADGESCSASMMYQEGKFRYRRVAE |
| | | | | GTQVLELPFKGDDITMVLILPKPEKSLAKVEKE |
| | | | | LTPEVLQEWLDELEEMMLVVHMPRFRIEDGFS |
| | | | | LKEQLQDMGLVDLFSPEKSKLPGIVAEGRDDLY |
| | | | | VSDAFHKAFLEVNEEGSEAAASTAVVIAGRSLN |
| | | | | PNRVTFKANRPFLVFIREVPLNTIIFMGRVANPC |
| | | | | VK |
| 17 | CO8A | Complement | P07357 | MFAVVFFILSLMTCQPGVTAQEKVNQRVRRAA |
| | | Component C8A Chain | | TPAAVTCQLSNWSEWTDCFPCQDKKYRHRSLL |
| | | | | QPNKFGGTICSGDIWDQASCSSSTTCVRQAQCG |
| | | | | QDFQCKETGRCLKRHLVCNGDQDCLDGSDED |
| | | | | DCEDVRAIDEDCSQYEPIPGSQKAALGYNILTQ |
| | | | | EDAQSVYDASYYGGQCETVYNGEWRELRYDS |
| | | | | TCERLYYGDDEKYFRKPYNFLKYHFEALADTGI |
| | | | | SSEFYDNANDLLSKVKKDKSDSFGVTIGIGPAG |
| | | | | SPLLVGVGVSHSQDTSFLNELNKYNEKKFIFTRI |
| | | | | FTKVQTAHFKMRKDDIMLDEGMLQSLMELPD |
| | | | | QYNYGMYAKFINDYGTHYITSGSMGGIYEYILV |
| | | | | IDKAKMESLGITSRDITTCFGGSLGIQYEDKINV |
| | | | | GGGLSGDHCKKFGGGKTERARKAMAVEDIISR |
| | | | | VRGGSSGWSGGLAQNRSTITYRSWGRSLKYNP |
| | | | | VVIDFEMQPIHEVLRHTSLGPLEAKRQNLRRAL |
| | | | | DQYLMEFNACRCGPCFNNGVPILEGTSCRCQCR |
| | | | | LGSLGAACEQTQTEGAKADGSWSCWSSWSVC |
| | | | | RAGIQERRRECDNPAPQNGGASCPGRKVQTQA |
| | | | | C |

Table 6 identifies and defines the glycan structures included in Table 1. Table 6 identifies a coded representation of the composition for each glycan structure included in Table 1. As used herein, the 4-digit GL NO. is a designation that represents the number of hexoses, the number of HexNAcs, the number of Fucoses, and the number of Neuraminic Acids.

TABLE 6
Glycan Structure GL NOS: Composition

| Glycan | | |
|---|---|---|
| Struc- | | |
| ture | | |
| GL | | Glycan |
| NO. | Glycan Symbol Structure | Composition |
| 3410 | 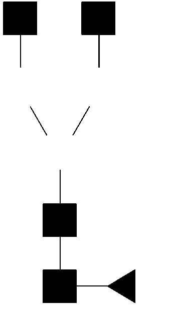 | Hex(3)HexNAc(4) Fuc(1)NeuAc(0) |
| 3510 | 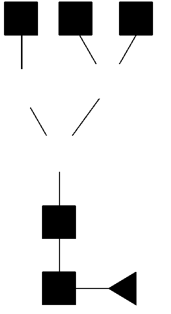 | Hex(3)HexNAc(5) Fuc(1)NeuAc(0) |
| | 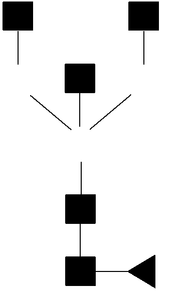 | |
| 4310 | 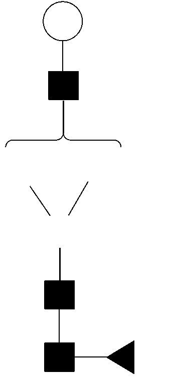 | Hex(4)HexNAc(3) Fuc(1)NeuAc(0) |
| 4400 | 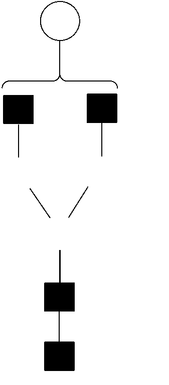 | Hex(4)HexNAc(4) Fuc(0)NeuAc(0) |
| 4410 | 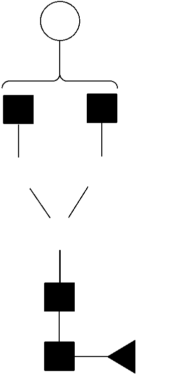 | Hex(4)HexNAc(4) Fuc(1)NeuAc(0) |
| 4411 | 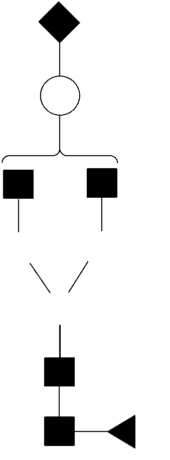 | Hex(4)HexNAc(4) Fuc(1)NeuAc(1) |
| 4510 | 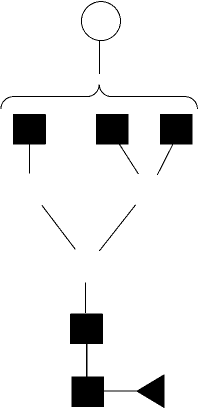 | Hex(4)HexNAc(5) Fuc(1)NeuAc(0) |
| | 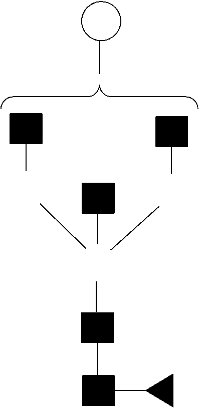 | |
| 5400 | 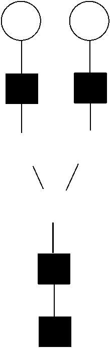 | Hex(5)HexNAc(4) Fuc(0)NeuAc(0) |
| | 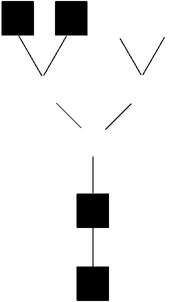 | |
| 5401 | 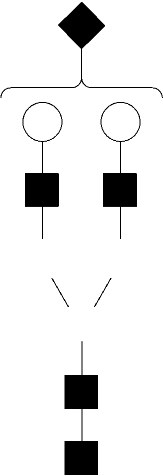 | Hex(5)HexNAc(4) Fuc(0)NeuAc(1) |
| 5402 | 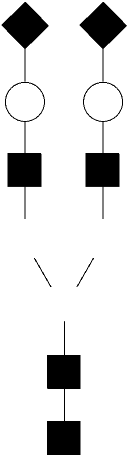 | Hex(5)HexNAc(4) Fuc(0)NeuAc(2) |
| 5410 | 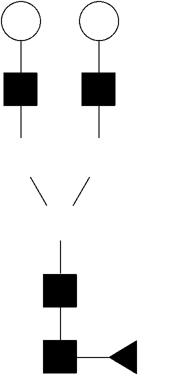 | Hex(5)HexNAc(4) Fuc(1)NeuAc(0) |
| 5411 | 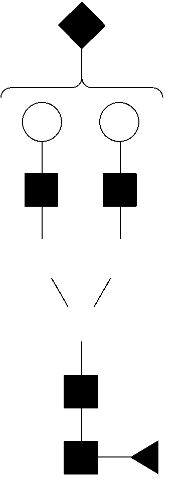 | Hex(5)HexNAc(4) Fuc(1)NeuAc(1) |
| 5510 | 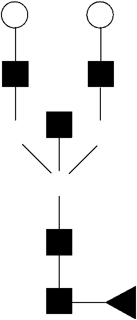 | Hex(5)HexNAc(5) Fuc(1)NeuAc(0) |
| 6502 | 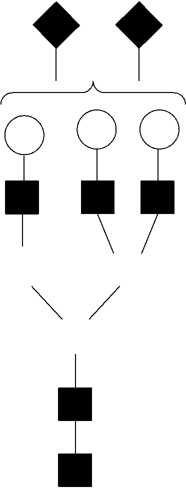 | Hex(6)HexNAc(5) Fuc(0)NeuAc(2) |
| 6503 | 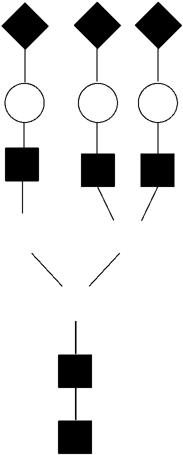 | Hex(6)HexNAc(5) Fuc(0)NeuAc(3) |
| 7602 | 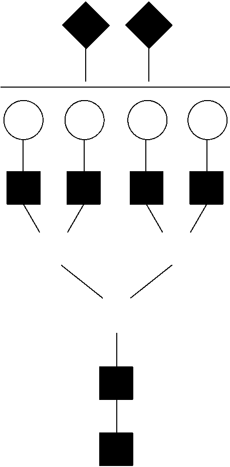 | Hex(7)HexNAc(6) Fuc(0)NeuAc(2) |
| 7613 | 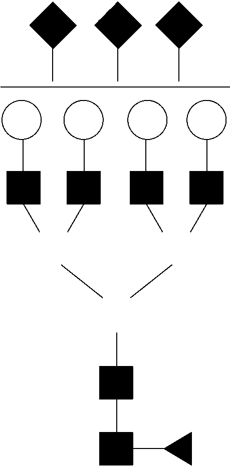 | Hex(7)HexNAc(6) Fuc(1)NeuAc(3) |
| 9804 | 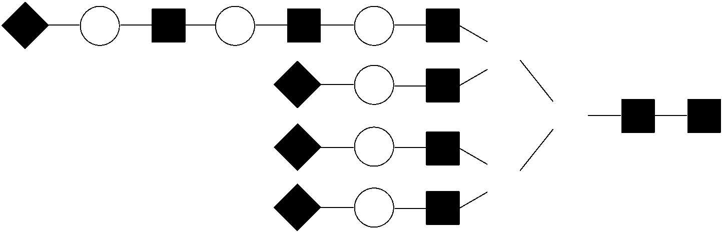 | Hex(9)HexNAc(8) Fuc(0)NeuAc(4) |
| 11904 | 6502 | 6502: |
| (6502 + 5402) | 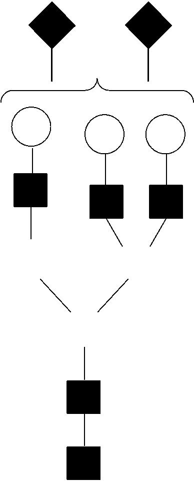 | Hex(6)HexNAc(5) Fuc(0)NeuAc(2) 5402: Hex(5)HexNAc(4) Fuc(0)NeuAc(2) |
| | 5402 | |
| | 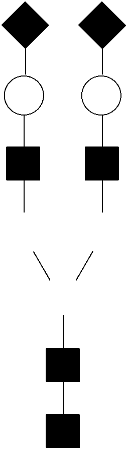 | |
| 11915 | 6513 | 6513: |
| (6513 + 5402) | 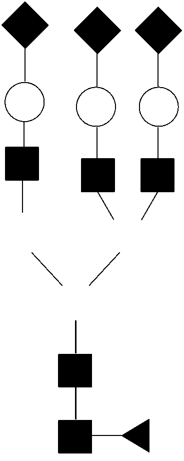 | Hex(6)HexNAc(5) Fuc(1)NeuAc(3) 5402: Hex(5)HexNAc(4) Fuc(0)NeuAc(2) |
| | 5402 | |
| | 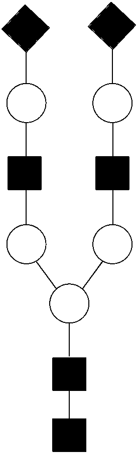 | |
| 121005 | 6502 | 6502: |
| | 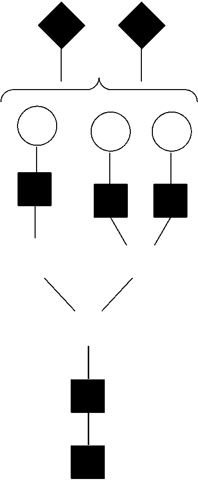 | Hex(6)HexNAc(5) Fuc(0)NeuAc(2) 6503: Hex(6)HexNAc(5) Fuc(0)NeuAc(3) |
| | 6503 | |
| | 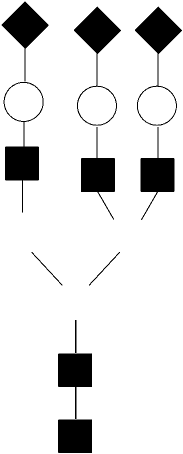 | |

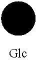

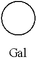

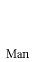

[symbol]

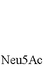

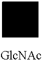

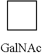

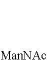

Table 6 illustrates the symbol structure and composition of detected glycan moieties that correspond to glycopeptides of Table 1, based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine. All of the glycans in Table 6 represent N-linked glycans.

For some entries, there are two symbol structures provided for one Glycan Structure GL NO such as, for example, Glycan Structure GL NO 5400 in Table 6. Thus, the identity of a peptide that references a Glycan Structure GL NO that has two symbol structures could be one of two possibilities based on the MRM of the LC-MS analysis.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

In some instances, a bracket symbol is used as part of the Symbol Structure (e.g., 4310) to indicate that the precise bonding linkage is not exactly known, but that the linking line segment is attached to one of the plurality of adjacent carbohydrates immediately adjacent to the bracket.

The identity of the various monosaccharides is illustrated by the Legend section located at the end of Table 6. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating a PC disease state. A transition includes a precursor ion and at least one product ion grouping. As reviewed herein, the peptide structures in Table 1, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 3 or an m/z ratio within an identified m/z ratio as provided in Table 3. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VIII. Group I Representative Experimental Results

### VIII.A. Subject Sample Cohort

To assess the association of individual peptide structures (biomarkers) with pancreatic cancer, three differential expression analyses (DEAs) were run on three different subject cohorts, adjusting for age and sex.
Cohort #1—First Differential Expression Analysis: The subject cohort (Cohort #1) for the first differential expression analysis included 50 subjects diagnosed with pancreatic cancer and 20 control subjects diagnosed as benign (e.g., a benign mass at a site other than the pancreas). The data for Cohort #1 was obtained from Indivumed GmbH (commercial biobank). Table 7A below identifies the fold changes, FDRs, and p-values as determined by the differential expression analysis (DEA) performed for Cohort #1.
Cohort #2—Second Differential Expression Analysis: The subject cohort (Cohort #2) for the second differential expression analysis included 45 subjects diagnosed with pancreatic cancer and 47 subjects diagnosed with benign pancreatitis. The data for Cohort #2 was obtained from Indivumed GmbH. Table 7B below identifies the fold changes, FDRs, and p-values as determined by the differential expression analysis (DEA) performed for Cohort #2.
Cohort #3—Third Differential Expression Analysis: The subject cohort (Cohort #3) for the third differential expression analysis included 113 subjects diagnosed with pancreatic cancer and 113 subjects diagnosed as healthy and matched to the subjects diagnosed with pancreatic cancer with respect to age and sex. Of the 113 subjects diagnosed with pancreatic cancer, 95 were also used on Cohorts #1 and #2. The data for Cohort #3 was obtained from Indivumed GmbH, from an academic institution, and iSpecimen (commercial biobank). Table 7C below identifies the fold changes, FDRs, and p-values as determined by the differential expression analysis (DEA) performed for Cohort #3.

These three different differential expression analyses were run for various peptide structures (e.g., hundreds of different peptide structures). Tables 7A-7C provide the statistical results (e.g., false discovery rates (FDRs), fold changes, p-values) for these analyses for the 38 peptide structure markers identified in Table 1. These 38 peptide structure markers were determined to be highly relevant markers for diagnosing pancreatic cancer. For the purposes of these three differential expression analyses, any quantification data for peptide structures PS-6 and PS-7 were treated as being for the same marker and thus these two peptide structures were considered as a single marker (DEA Marker Index 6). Further, any quantification data for peptide structures PS-12 and PS-13 were treated as being for the same marker and thus these two peptide structures were considered as a single marker (DEA Marker Index 11). Thus, the 38 markers identified in Table 1 form 36 markers for these analyses.

TABLE 7A
First Differential Expression Analysis (DEA) - Cohort #1

| DEA | | | | | |
|---|---|---|---|---|---|
| Marker | PS-ID | | PC/Control | PC/Control | PC/Control |
| Index | NO. | PS-NAME | (fold change) | (FDR) | (p-value) |
| 1 | PS-1 | APOA1_DLATVYVDVLK | 0.55844 | 1.70E−05 | 8.41E−07 |
| 2 | PS-2 | A2MG_55_5402 | 1.17227 | 0.00305 | 0.00052 |
| 3 | PS-3 | HRG_125_5402 | 1.16310 | 0.03567 | 0.01170 |
| 4 | PS-4 | HPT_207_121005 | 0.49842 | 0.00038 | 3.35E−05 |
| 5 | PS-5 | HPT_207_11904 | 0.60973 | 1.20E−05 | 4.53E−07 |
| 6 | PS-6 | AGP1_72MC_6503 | 0.60117 | 1.42E−08 | 8.98E−07 |
| | and/or | and/or | | | |
| | PS-7 | AGP2_72MC_6503 | | | |
| 7 | PS-8 | A2MG_869_5402 | 1.17796 | 0.01396 | 0.00366 |
| 8 | PS-9 | A1AT_AVLTIDEK | 1.28093 | 0.00642 | 0.00136 |
| 9 | PS-10 | AACT_271_7602 | 0.49408 | 3.73E−05 | 2.45E−06 |
| 10 | PS-11 | HPT_241_7613 | 1.95269 | 0.00507 | 0.00100 |
| 11 | PS-12 | HEMO_240_5402 | 0.93665 | 0.54792 | 0.41203 |
| | and/or | and/or | | | |
| | PS-13 | HEMO_246_5402 | | | |
| 12 | PS-14 | TRFE_432_6503 | 1.22621 | 0.00778 | 0.00171 |
| 13 | PS-15 | IGJ_71_5401 | 0.96124 | 0.54421 | 0.4071 |
| 14 | PS-16 | CFAH_882_5401 | 0.73652 | 0.01270 | 0.00317 |
| 15 | PS-17 | IGA2_205_5410 | 0.88594 | 0.64645 | 0.50792 |
| 16 | PS-18 | IGG1_297_3510 | 2.51483 | 0.01375 | 0.00357 |
| 17 | PS-19 | IGG1_297_3410 | 2.75367 | 0.00362 | 0.00065 |
| 18 | PS-20 | FETUA_156_6503 | 0.89517 | 0.39357 | 0.25145 |
| 19 | PS-21 | IGG1_297_4400 | 2.69628 | 0.00421 | 0.00079 |
| 20 | PS-22 | IGG1_297_4410 | 2.98789 | 0.00134 | 0.00017 |
| 21 | PS-23 | IGG1_297_4410 | 2.97583 | 0.00150 | 0.00021 |
| 22 | PS-24 | IGG1_297_4411 | 3.08443 | 0.00159 | 0.00023 |
| 23 | PS-25 | IGG1_297_4510 | 2.39336 | 0.01266 | 0.00310 |
| 24 | PS-26 | HPT_184_5401 | 0.81917 | 0.34059 | 0.20949 |
| 25 | PS-27 | HPT_207_11915 | 1.89585 | 1.16E−06 | 2.76E−08 |
| 26 | PS-28 | IGG1_297_{—} | 2.21258 | 0.02478 | 0.00738 |
| | | NLFLNHSENATAK | | | |
| 27 | PS-29 | IGG1_297_5510 | 2.30401 | 0.01755 | 0.00484 |
| 28 | PS-30 | IGG1_297_5411 | 2.64102 | 0.00349 | 0.00062 |
| 29 | PS-31 | IGG1_297_5410 | 2.51676 | 0.00598 | 0.00126 |
| 30 | PS-32 | IGG1_297_5400 | 2.19740 | 0.02383 | 0.00698 |
| 31 | PS-33 | FETUA_176_6502 | 1.13204 | 0.46148 | 0.31589 |
| 32 | PS-34 | IGM_46_4310 | 1.21069 | 0.03577 | 0.01195 |
| 33 | PS-35 | AGP1_93_7613 | 2.19059 | 3.03E−07 | 2.40E−09 |
| 34 | PS-36 | ANT_187_5402 | 0.94438 | 0.26795 | 0.15364 |
| 35 | PS-37 | COSA_LYYGDDEK | 0.97915 | 0.82181 | 0.72361 |
| 36 | PS-38 | AGP1_103_9804 | 0.63995 | 0.00017 | 1.28E−05 |

TABLE 7B
Second Differential Expression Analysis (DEA) - Cohort #2

| DEA | | | | | |
|---|---|---|---|---|---|
| Marker | PS-ID | | PC/Control | PC/Control | PC/Control |
| Index | NO. | PS-NAME | (fold change) | (FDR) | (p-value) |
| 1 | PS-1 | APOA1_DLATVYVDVLK | 0.76495 | 0.01812 | 0.00048 |
| 2 | PS-2 | A2MG_55_5402 | 1.11600 | 0.06102 | 0.00512 |
| 3 | PS-3 | HRG_125_5402 | 1.15342 | 0.04515 | 0.00312 |
| 4 | PS-4 | HPT_207_121005 | 0.73188 | 0.04434 | 0.00199 |
| 5 | PS-5 | HPT_207_11904 | 0.82486 | 0.07209 | 0.00800 |
| 6 | PS-6 | AGP1_72MC_6503 | 0.80878 | 0.04515 | 0.00274 |
| | and/or | and/or | | | |
| | PS-7 | AGP2_72MC_6503 | | | |
| 7 | PS-8 | A2MG_869_5402 | 1.12274 | 0.25151 | 0.05649 |
| 8 | PS-9 | A1AT_AVLTIDEK | 1.14883 | 0.14858 | 0.02554 |
| 9 | PS-10 | AACT_271_7602 | 0.70837 | 0.04515 | 0.00222 |
| 10 | PS-11 | HPT_241_7613 | 1.63174 | 0.04515 | 0.00231 |
| 11 | PS-12 | HEMO_240_5402 | 1.01127 | 0.96115 | 0.90108 |
| | and/or | and/or | | | |
| | PS-13 | HEMO_246_5402 | | | |
| 12 | PS-14 | TRFE_432_6503 | 0.93353 | 0.77816 | 0.55624 |
| 13 | PS-15 | IGJ_71_5401 | 0.93459 | 0.39408 | 0.14030 |
| 14 | PS-16 | CFAH_882_5401 | 0.84053 | 0.04515 | 0.00317 |
| 15 | PS-17 | IGA2_205_5410 | 1.36726 | 0.33578 | 0.10035 |
| 16 | PS-18 | IGG1_297_3510 | 1.98850 | 0.04515 | 0.00242 |
| 17 | PS-19 | IGG1_297_3410 | 4.62462 | 0.01812 | 0.00055 |
| 18 | PS-20 | FETUA_156_6503 | 0.87001 | 0.29479 | 0.07773 |
| 19 | PS-21 | IGG1_297_4400 | 2.18496 | 0.01812 | 0.00041 |
| 20 | PS-22 | IGG1_297_4410 | 2.42580 | 0.01812 | 0.00011 |
| 21 | PS-23 | IGG1_297_4410 | 2.13286 | 0.01812 | 0.00028 |
| 22 | PS-24 | IGG1_297_4411 | 2.11467 | 0.01812 | 0.00055 |
| 23 | PS-25 | IGG1_297_4510 | 2.18227 | 0.01812 | 0.00045 |
| 24 | PS-26 | HPT_184_5401 | 0.79626 | 0.58562 | 0.31984 |
| 25 | PS-27 | HPT_207_11915 | 1.23475 | 0.04515 | 0.00306 |
| 26 | PS-28 | IGG1_297_{—} | 2.19984 | 0.01812 | 0.00027 |
| | | NLFLNHSENATAK | | | |
| 27 | PS-29 | IGG1_297_5510 | 2.42830 | 0.01812 | 0.00043 |
| 28 | PS-30 | IGG1_297_5411 | 2.33570 | 0.01812 | 0.00016 |
| 29 | PS-31 | IGG1_297_5410 | 2.23586 | 0.01812 | 0.00036 |
| 30 | PS-32 | IGG1_297_5400 | 2.44643 | 0.01812 | 0.00050 |
| 31 | PS-33 | FETUA_176_6502 | 1.32721 | 0.56553 | 0.28277 |
| 32 | PS-34 | IGM_46_4310 | 1.29170 | 0.32762 | 0.09214 |
| 33 | PS-35 | AGP1_93_7613 | 1.48180 | 0.03724 | 0.00145 |
| 34 | PS-36 | ANT_187_5402 | 0.98451 | 0.91417 | 0.80410 |
| 35 | PS-37 | COSA_LYYGDDEK | 1.27293 | 0.07578 | 0.00873 |
| 36 | PS-38 | AGP1_103_9804 | 1.13855 | 0.70578 | 0.44662 |

TABLE 7C
Third Differential Expression Analysis (DEA)- Cohort #3

| DEA | | | | | |
|---|---|---|---|---|---|
| Marker | PS-ID | | PC/Control | PC/Control | PC/Control |
| Index | NO. | PS-NAME | (fold change) | (FDR) | (p-value) |
| 1 | PS-1 | APOA1_DLATVYVDVLK | 0.58995 | 1.40E−26 | 5.57E−29 |
| 2 | PS-2 | A2MG_55_5402 | 1.32128 | 3.19E−22 | 2.54E−24 |
| 3 | PS-3 | HRG_125_5402 | 1.31537 | 8.52E−15 | 1.19E−16 |
| 4 | PS-4 | HPT_207_121005 | 0.56743 | 1.76E−14 | 2.81E−16 |
| 5 | PS-5 | HPT_207_11904 | 0.69855 | 1.80E−13 | 3.94E−15 |
| 6 | PS-6 | AGP1_72MC_6503 | 0.74369 | 2.47E−10 | 1.13E−11 |
| | and/or | and/or | | | |
| | PS-7 | AGP2_72MC_6503 | | | |
| 7 | PS-8 | A2MG_869_5402 | 1.24918 | 5.92E−09 | 4.05E−10 |
| 8 | PS-9 | A1AT_AVLTIDEK | 1.31821 | 3.62E−08 | 3.10E−09 |
| 9 | PS-10 | AACT_271_7602 | 0.57160 | 8.60E−07 | 1.08E−07 |
| 10 | PS-11 | HPT_241_7613 | 1.86097 | 1.06E−06 | 1.35E−07 |
| 11 | PS-12 | HEMO_240_5402 | 0.45589 | 1.11E−06 | 1.44E−07 |
| | and/or | and/or | | | |
| | PS-13 | HEMO_246_5402 | | | |
| 12 | PS-14 | TRFE_432_6503 | 1.25215 | 3.71E−06 | 5.17E−07 |
| 13 | PS-15 | IGJ_71_5401 | 0.84697 | 6.84E−06 | 1.03E−06 |
| 14 | PS-16 | CFAH_882_5401 | 0.77999 | 1.15E−05 | 1.80E−06 |
| 15 | PS-17 | IGA2_205_5410 | 1.78628 | 1.38E−05 | 2.19E−06 |
| 16 | PS-18 | IGG1_297_3510 | 2.83089 | 2.08E−05 | 3.43E−06 |
| 17 | PS-19 | IGG1_297_3410 | 2.64003 | 2.08E−05 | 3.47E−06 |
| 18 | PS-20 | FETUA_156_6503 | 0.78847 | 2.49E−05 | 4.25E−06 |
| 19 | PS-21 | IGG1_297_4400 | 2.45814 | 2.86E−05 | 5.00E−06 |
| 20 | PS-22 | IGG1_297_4410 | 2.58305 | 4.48E−05 | 8.26E−06 |
| 21 | PS-23 | IGG1_297_4410 | 2.59805 | 4.74E−05 | 8.86E−06 |
| 22 | PS-24 | IGG1_297_4411 | 2.69411 | 5.00E−05 | 9.65E−06 |
| 23 | PS-25 | IGG1_297_4510 | 2.43876 | 0.00011 | 2.42E−05 |
| 24 | PS-26 | HPT_184_5401 | 0.75597 | 0.00016 | 3.61E−05 |
| 25 | PS-27 | HPT_207_11915 | 1.24303 | 0.00025 | 6.09E−05 |
| 26 | PS-28 | IGG1_297_{—} | 2.16947 | 0.00044 | 0.00012 |
| | | NLFLNHSENATAK | | | |
| 27 | PS-29 | IGG1_297_5510 | 2.19925 | 0.00055 | 0.00015 |
| 28 | PS-30 | IGG1_297_5411 | 2.27010 | 0.00059 | 0.00016 |
| 29 | PS-31 | IGG1_297_5410 | 2.17298 | 0.00099 | 0.00028 |
| 30 | PS-32 | IGG1_297_5400 | 2.08028 | 0.00154 | 0.00047 |
| 31 | PS-33 | FETUA_176_6502 | 1.99290 | 0.01063 | 0.00395 |
| 32 | PS-34 | IGM_46_4310 | 1.11865 | 0.05347 | 0.02434 |
| 33 | PS-35 | AGP1_93_7613 | 2.44732 | 0.11505 | 0.06039 |
| 34 | PS-36 | ANT_187_5402 | 0.92549 | 0.19127 | 0.11522 |
| 35 | PS-37 | COSA_LYYGDDEK | 1.06107 | 0.24711 | 0.15623 |
| 36 | PS-38 | AGP1_103_9804 | 1.05395 | 0.91615 | 0.88883 |

### VIII.B. Training a Binary Classification Model

A full panel of biomarkers were included in training a binary classification model for diagnosing pancreatic cancer status. For Cohort #3, the total number of subjects was split into 70% training (n=159) and 30% testing (n=67). For the training set, repeated, 10-fold cross-validation was used to select optimal hyperparameters for LASSO, and then these hyperparameters were used on the entire training set develop one predictive logistic regression model. This model was then blindly used to predict pancreatic cancer status in the test set. Overall, 19 markers were left with non-zero weights after LASSO shrinkage. These 19 markers are identified in Table 2 above. The 36 markers identified in Tables 7A-7C above include the 19 markers identified via LASSO and 17 additional markers having FDR<0.05 and concordant directions of effect.

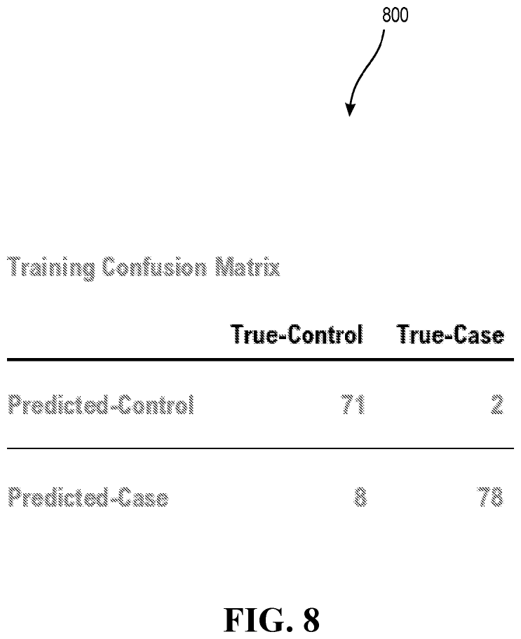

FIG. 8 is a confusion matrix for the model for the training set in accordance with one or more embodiments. Confusion matrix 800 illustrates that the model was able to correctly predict that 71 subjects had pancreatic cancer out of the total 79 subjects in the training set diagnosed with pancreatic cancer. Confusion matrix 800 further illustrates that the model was able to correctly predict that 78 subjects did not have pancreatic cancer out of the total 80 subjects in the training set diagnosed as healthy.

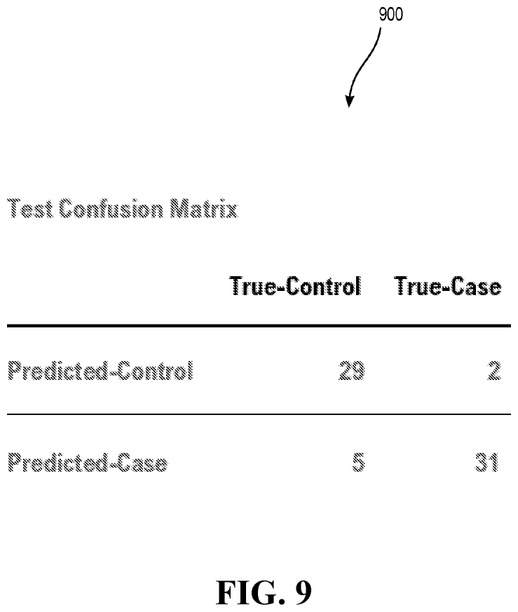

FIG. 9 is a confusion matrix for the model for the testing set in accordance with one or more embodiments. Confusion matrix 800 illustrates that the model was able to correctly predict that 29 subjects had pancreatic cancer out of the total 34 subjects in the testing set diagnosed with pancreatic cancer. Confusion matrix 800 further illustrates that the model was able to correctly predict that 31 subjects did not have pancreatic cancer out of the total 33 subjects in the testing set diagnosed as healthy.

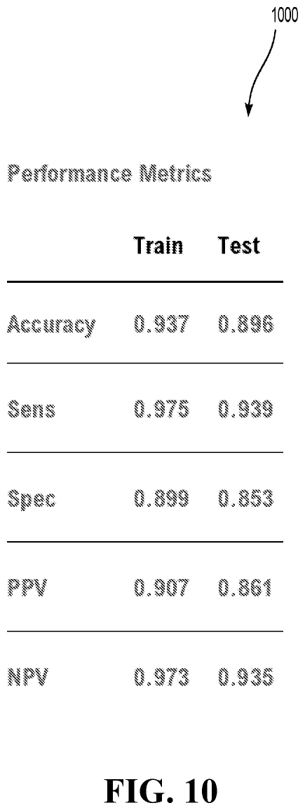

FIG. 10 is a table describing performance metrics for the model for the training and testing sets in accordance with one or more embodiments. Table 1000 includes the accuracy, sensitivity, specificity, positive predictive value (e.g., probability of the presence of pancreatic cancer given a positive test result), and negative predictive value (e.g., probability of the absence of disease given a negative test result) for the model.

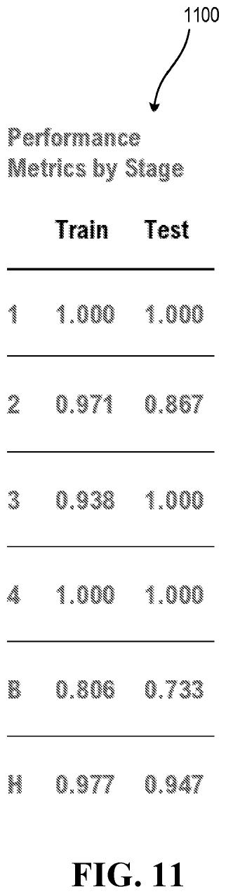

FIG. 11 is a table describing performance metrics by stage of pancreatic cancer. Table 1100 includes the accuracy of the model in predicting pancreatic cancer for the various stages (e.g., 1, 2, 3, and 4) associated with pancreatic cancer as well as a healthy state and a benign state. The benign state represents the presence of at least one benign mass, which may be located in or on the pancreas and/or some other location within the body.

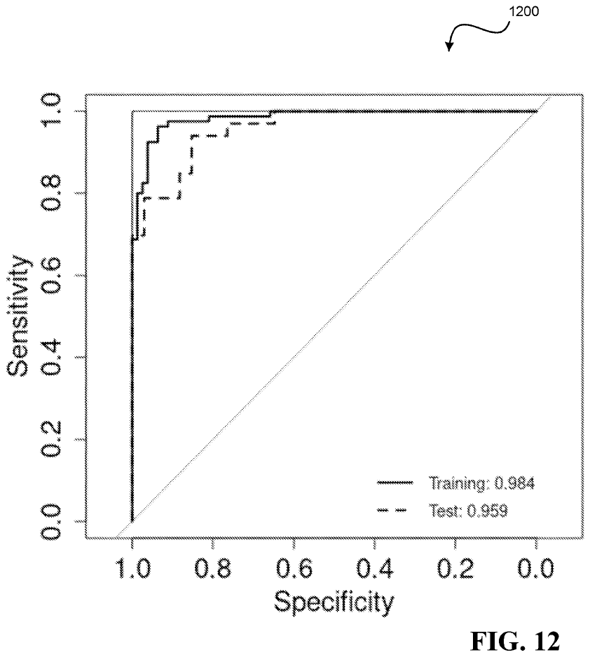

FIG. 12 is a plot of a receiver operating characteristic (ROC) curve for the model for the training set and testing set in accordance with one or more embodiments. Plot 1200 illustrates specificity versus sensitivity. The area under the curve (AUC) for the training set was found to be 0.984 and the AUC for the testing set was found to be 0.959.

### IX. Exemplary Methodologies Relating to Diagnosis Based On Peptide Structure Data Analysis-Group II

### IX.A. General Methodology

As noted above in Section VIIA, FIG. 5 is a flowchart of a process for diagnosing a subject with respect to a pancreatic cancer (PC) disease state in accordance with one or more embodiments, and it may be applied to different sets of peptide structure data obtained from a subject or subjects, such as that related to Group II set of peptide structure data. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

### IX.B. Process 500 Diagnosis Using Tables 8-14

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 8, with the peptide sequence being one of SEQ ID NOS: 18, 21, 25, 28, 32, or 51-67 as defined in Table 8.

Step 504 includes analyzing the peptide structure data using a supervised machine learning model to generate a disease indicator that indicates whether the biological sample evidences a PC disease state based on at least 3 peptide structures selected from a group of peptide structures identified in Table 8 (below). In step 504, the group of peptide structures in Table 8 is associated with the PC disease state. The group of peptide structures is listed in Table 8 with respect to relative significance to the disease indicator.

In one or more embodiments, the at least 3 peptide structures includes at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-1 to PS-22 in Table 8.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is positive for the PC disease state and the supervised machine learning model is configured to generate an output that identifies the biological sample as either evidencing (“positive for”) the PC disease state when the disease indicator is greater than a selected threshold or not evidencing (“negative for”) the PC disease state when the disease indicator is not greater than the selected threshold. The selected threshold may be, for example, 0.30, 0.35, 0.40, 0.45, 0.50, 0.55, or some other threshold. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the PC disease state if the biological sample evidences the PC disease state based on the disease indicator. The diagnosis may be, for example, “negative” if the biological sample does not evidence the PC disease state based on the disease indicator. A negative diagnosis may mean that the biological sample has a non-pancreatic cancer (PC) state (e.g., healthy, control, etc.). The negative diagnosis for the PC disease state can include at least one of a healthy state, a benign pancreatitis state, or a control state.

Generating the diagnosis output in step 506 may include determining that the score falls above a selected threshold and generating a positive diagnosis for the PC disease state. Alternatively, step 506 can include determining that the score falls below a selected threshold and generating a negative diagnosis for the PC disease state. In some scoring systems, the score can include a probability score and the selected threshold can be 0.5. In other scoring systems, the selected threshold can fall within a range between 0.4 and 0.6.

In one or more embodiments, the final output in step 506 may include a treatment output if the diagnosis output indicates a positive diagnosis for the PC disease state. The treatment output may include, for example, at least one of an identification of a treatment for the subject, a treatment plan for administering the treatment, or both. Treatment for pancreatic cancer may include, for example, but is not limited to, at least one of radiation therapy, chemoradiotherapy, surgery, a targeted drug therapy, immunotherapy, chemotherapy, or some other form of treatment. The treatment plan may include, for example, but is not limited to, a timeline or schedule for administering the treatment, dosing information, other treatment-related information, or a combination thereof. Chemotherapy may comprise one or more of Gemcitabine, Nab-paclitaxel, 5-fluorouracil (F-5U), Irinotecan, Oxaliplatin, Capecitabine, Cisplatin, and Liposomal Irinotecan. In specific embodiments, the chemotherapy comprises (1) Gemcitabine plus nab-paclitaxel, and/or (2) 5-FU, irinotecan, and oxaliplatin. In specific cases, the patient is provide up to two dose reductions for nab-paclitaxel (to 100 mg/m² and 75 mg/m²) and gemcitabine (to 800 mg/m² and 600 mg/m²).

TABLE 8
Group II Peptide Structures associated with Pancreatic Cancer

| | | | | Linking | Linking | | |
|---|---|---|---|---|---|---|---|
| | | Prot | Pept | Site | Site | Glycan | Monoisotop. |
| PS-ID | Pept Structure | SEQ | SEQ | Pos. in | Pos. in | Struct | mass |
| NO. | (PS)-NAME | ID NO. | ID NO. | Prot Seq | Pept Seq | GL NO. | GlyPep_MW |
| PS-21 | TRFE_432_5401 | 10 | 28 | 432 | 12 | 5401 | 3389.421198 |
| PS-5 | IC1_352_5402 | 42 | 54 | 352 | 9 | 5402 | 4517.13034 |
| PS-19 | APOM_AFLLTPR | 49 | 66 | N/A | N/A | N/A | 816.485756 |
| PS-20 | APOA1_DLATVYVDVLK | 1 | 18 | N/A | N/A | N/A | 1234.680878 |
| PS-22 | TTR_TSESGELHGLTTEEE | 50 | 67 | N/A | N/A | N/A | 2454.143774 |
| | FVEGIYK | | | | | | |
| PS-2 | A2GL_DLLLPQPDLR | 41 | 52 | N/A | N/A | N/A | 1178.665896 |
| PS-6 | IC1_238_5412 | 42 | 55 | 238 | 6 | 5412 | 3259.26547 |
| PS-17 | A2MG_247_5200 | 2 | 64 | 247 | 10 | 5200 | 4950.310912 |
| PS-3 | A1AT_107_6512 | 7 | 53 | 107 | 14 | 6512 | 6406.77897 |
| PS-15 | IGG2_297_3500 | 46 | 62 | 176 | 5 | 3500 | 2658.070174 |
| PS-1 | AGP12_56_5412 | 5 or 6 | 51 | 56 | 5 | 5412 | 3146.170176 |
| PS-4 | HPT_207_121015 | 4 | 21 | 207, 211 | 5, 9 | 6502 | 7034.688732 |
| | | | | | | or | |
| | | | | | | 6513 | |
| PS-7 | AACT_271_6512 | 8 | 25 | 271 | 4 | 6512 | 4467.835462 |
| PS-14 | A1AT_107_nonglycosylated | 7 | 61 | N/A | N/A | N/A | 3690.816484 |
| PS-16 | C1S_174_5402 | 47 | 63 | 174 | 5 | 5402 | 5730.401612 |
| PS-12 | IGM_439_9200 | 15 | 59 | 440 | 9 | 9200 | 4228.73181 |
| PS-18 | FINC_SYTITGLQPGTDYK | 48 | 65 | N/A | N/A | N/A | 1542.756554 |
| PS-13 | IC1_253_6503 | 42 | 60 | 253 | 4 | 6503 | 4961.085074 |
| PS-11 | AGP12_72_7601 | 5 or 6 | 58 | 72 | 15 | 7601 | 4562.887832 |
| PS-10 | B2M_VNHVTLSQPK | 45 | 57 | N/A | N/A | N/A | 1121.61928 |
| PS-9 | IGA12_144_3500 | 44 or 13 | 56 | 144 | 18 | 3500 | 4464.14622 |
| | | | | (P01876) or | | | |
| | | | | 131 | | | |
| | | | | (P01877) | | | |
| PS-8 | IGG1_297_3510 | 43 | 32 | 180 | 5 | 3510 | 2836.117908 |

As with Table 1 for Group I peptide structures above, Table 8 includes the Peptide Structure Identification Number (PS-ID NO.) that is a reference number for a particular peptide or glycopeptide. The Peptide Structure Name (PS-Name, e.g., AGP12_56_5412), which is a reference code for the protein name (e.g., AGP12), followed by the glycan linking site position in the protein (e.g., the number 56 that is in between two underscores and represents a sequential amino acid position in protein AGP12), and followed by the glycan structure GL number (e.g., the number 5412 that is preceded by an underscore and represents a glycan composition Hex(5)HexNAc(4)Fuc(1)NeuAc(2)). The Protein Sequence ID No of Table 8 corresponds to the corresponding protein name, and Uniprot ID of Table 12. The Peptide Sequence ID No of Table 8 respectively corresponds to the corresponding peptide sequence of Table 11. The term Linking Site Pos. within Protein Sequence is a number that refers to the sequential position of an amino acid of the corresponding protein in which a glycan is attached. For the Glycan Linking Site Pos. within Protein Sequence, the amino acid position of the peptide sequence is defined by the sequentially numbered order of amino acids based on the Uniprot ID of the corresponding protein for the peptide sequence. The term Linking Site Pos. within Peptide Sequence is a number that refers to the sequential position of an amino acid of the corresponding peptide in which a glycan is attached. For the Glycan Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Table 13.

With respect to marker HPT_207_121015 (PS-4), the peptide structure has two linking site positions and two glycan structure GL NOs. because there are two glycosylation sites in that peptide sequence. Hence, glycan 6502 (which is composition Hex(6)HexNAc(5)Fuc(0)NeuAc(2)) is linked to position 207, and glycan structure 6513 (which is composition Hex(6)HexNAc(5)Fuc(1)NeuAc(3)) is linked to position 211.

IX.C. Training the Model to Diagnose with Respect to the PC Disease State

With respect to Group II peptide structures, FIG. 6 is also a flowchart of a process for training a model to diagnose a subject with respect to a pancreatic cancer (PC) disease state in accordance with one or more embodiments. Process 600 may be implemented using, for example, at least a portion of workflow 100 as described in FIGS. 1, 2A, and 2B and/or analysis system 300 as described in FIG. 3. In some embodiments, process 600 may be one example of an implementation for training the model used in the process 500 in FIG. 5.

Step 602 includes receiving quantification data for a panel of peptide structures for a plurality of subjects. The plurality of subjects includes a first portion diagnosed with a negative diagnosis of a PC disease state and a second portion diagnosed with a positive diagnosis of the PC disease state. The quantification data comprises a plurality of peptide structure profiles for the plurality of subjects.

Step 604 includes training a machine learning model using the quantification data to diagnose a biological sample with respect to the PC disease state using a group of peptide structures associated with the PC disease state (e.g., the group of peptide structures is identified in Table 8). The group of peptide structures is listed in Table 8 with respect to relative significance to diagnosing the biological sample. Step 604 can include training the machine learning using a portion of the quantification data corresponding to a training group of peptide structures included in the plurality of peptide structures.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the PC disease state and a negative diagnosis for any subject of the plurality of subjects determined not to have the PC disease state.

The machine learning model can include a binary classification model. Some binary classification models can include logistical regression models. Some logistical regression models can include LASSO regression models.

An alternative or additional step in process 600 can include performing a differential expression analysis using initial training data to compare a first portion of the plurality of subjects diagnosed with the positive diagnosis for the PC disease state versus a second portion of the plurality of subjects diagnosed with the negative diagnosis for the PC disease state.

An alternative or additional step in process 600 can include identifying a training group of peptide structures based on the differential expression analysis for use as prognostic markers for the PC disease state.

An alternative or additional step in process 600 can include forming the training data based on the training group of peptide structures identified.

An alternative or additional step in process 600 can include identifying a training group of peptide structures based on the differential expression analysis, wherein the training group of peptide structures is a subset of the plurality of peptide structures relevant to diagnosing the PC disease state. The subset may be identified based on at least one of fold-changes, false discovery rates, or p-values computed as part of the differential expression analysis.

An alternative or additional step in process 600 can include training a machine learning model, using the quantification data for the training group of peptide structures, to diagnose a subject of a biological sample with respect to the PC disease state using a group of peptide structures associated with the PC disease state. The group of peptide structures may be a subset of the training group of peptide structures and is identified in Table 8. The group of peptide structures is listed in Table 8 with respect to relative significance to making the diagnosis.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

For example, the machine learning model may be a LASSO regression model that identifies the peptide structures of Table 9 below, which include at least a portion of the group of peptide structures identified in Table 8. The markers used for training of the LASSO regression model may, in one or more embodiments, additionally include one or more other peptide structure markers.

TABLE 9
Peptide Structures After LASSO Shrinkage

| | | (Protein) | (Peptide) |
|---|---|---|---|
| PS-ID | | SEQ | SEQ |
| NO. | PS-NAME | ID NO. | ID NO. |
| PS-21 | TRFE_432_5401 | 10 | 28 |
| PS-5 | IC1_352_5402 | 42 | 54 |
| PS-19 | QUANTPEP.APOM_AFLLTPR | 49 | 66 |
| PS-20 | APOA1_DLATVYVDVLK | 1 | 18 |
| PS-22 | TTR_TSESGELHGLTTEEEFVE | 50 | 67 |
| | GIYK | | |
| PS-2 | A2GL_DLLLPQPDLR | 41 | 52 |
| PS-6 | IC1_238_5412 | 42 | 55 |
| PS-17 | A2MG_247_5200 | 2 | 64 |
| PS-3 | A1AT_107_6512 | 7 | 53 |
| PS-15 | IGG2_297_3500 | 46 | 62 |
| PS-1 | AGP12_56_5412 | 5 or 6 | 51 |
| PS-4 | HPT_207_121015 | 4 | 21 |
| PS-7 | AACT_271_6512 | 8 | 25 |
| PS-14 | A1AT_107_nonglycosylated | 7 | 61 |
| PS-16 | C1S_174_5402 | 47 | 63 |
| PS-12 | IGM_439_9200 | 15 | 59 |
| PS-18 | FINC_SYTITGLQPGTDYK | 48 | 65 |
| PS-13 | IC1_253_6503 | 42 | 60 |
| PS-11 | AGP12_72_7601 | 5 or 6 | 58 |
| PS-10 | B2M_VNHVTLSQPK | 45 | 57 |
| PS-9 | IGA12_144_3500 | 44 or 13 | 56 |
| PS-8 | IGG1_297_3510 | 43 | 32 |

In one or more embodiments, a subset of the markers identified in Table 2 may be used for training of the LASSO regression model. Alternatively, the markers identified in Table 9 may be a subset for training of the LASSO regression model. For example, the LASSO regression model may be trained using at least one other marker in addition to those identified in Table 9.

### IX.D. Monitoring a Subject for a Pancreatic Cancer Disease State

FIG. 7 is a flowchart of a process for monitoring a subject for a pancreatic cancer (PC) disease state in accordance with one or more embodiments. Process 700 may be implemented using, for example, at least a portion of workflow 100 as described in FIGS. 1, 2A, and 2B and/or analysis system 300 as described in FIG. 3.

Step 702 includes receiving first peptide structure data for a first biological sample obtained from a subject at a first timepoint.

Step 704 includes analyzing the first peptide structure data using a supervised machine learning model to generate a first disease indicator based on at least 3 peptide structures selected from a group of peptide structures identified in Table 8. The group of peptide structures in Table 8 includes a group of peptide structures associated with a PC disease state in accordance with various embodiments. The supervised machine can be a binary classification model. In some embodiments, the binary classification model can be a logistical regression model.

Step 706 includes receiving second peptide structure data of a second biological sample obtained from the subject at a second timepoint.

Step 708 includes analyzing the second peptide structure data using the supervised machine learning model to generate a second disease indicator based on the at least 3 peptide structures selected from the group of peptide structures identified in Table 8.

Step 710 includes generating a diagnosis output based on the first disease indicator and the second disease indicator. Generating the diagnostic output can include comparing the second disease indicator to the first disease indicator.

In some embodiments, the first disease indicator indicates that the first biological sample evidences the negative diagnosis for the PC disease state and the second biological sample evidences the positive diagnosis for the PC disease. In other embodiments, the diagnosis output identifies whether a non-PC disease state has progressed to the PC disease state, wherein the non-PC disease state includes either a healthy state or a benign pancreatitis state.

### X. Group II Peptide Structure and Product Ion Compositions, Kits and Reagents

Aspects of the disclosure include compositions comprising one or more of the Group II peptide structures listed in Table 8. In some embodiments, a composition comprises a plurality of the peptide structures listed in Table 8. In some embodiments, a composition comprises 1, 2, 3, 4, 5, 6, 7, 8, 9, 10, 11, 12, 13, 14, 15, 16, 17, 18, 19, 20, 21, or 22 of the peptide structures listed in Table 8. In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 18, 21, 25, 28, 32, 51-67, listed in Table 8.

Aspects of the disclosure include compositions comprising one or more precursor ions having a defined charge and/or defined mass-to-charge (m/z) ratio, as listed in Table 10. Aspects of the disclosure include compositions comprising one or more product ions having a defined mass-to-charge (m/z) ratio, which product ions are produced by converting a peptide structure described herein (e.g., a peptide structure listed in Table 8) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Table 8). In some embodiments, a composition comprises a set of the product ions listed in Table 10, having an m/z ratio selected from the list provided for each peptide structure in Table 8.

In some embodiments, a composition comprises at least one of peptide structures PS-1 to PS-22 identified in Table 8. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, or all 22 of the peptide structures PS-1 to PS-22 in Table 8.

In some embodiments, a composition comprises a peptide structure or a product ion. In some embodiments, the peptide structure or product ion comprises an amino acid sequence having at least 90% sequence identity to any one of SEQ ID NOS: 18, 21, 25, 28, 32, 51-57, as identified in Table 4, corresponding to peptide structures PS-1 to PS-22 in Table 8.

In some embodiments, a composition comprises a peptide structure or a product ion. In some embodiments, the peptide structure or product ion comprises an amino acid sequence having at least 90% sequence identity to any one of SEQ ID NOS: 18, 21, 25, 28, 32, 51-57, as identified in Table 11, corresponding to peptide structures PS-1 to PS-22 in Table 8.

In some embodiments, the product ion is selected as one from a group consisting of product ions identified in Table 10, including product ions falling within an identified m/z range of the m/z ratio identified in Table 10 and characterized as having a precursor ion having an m/z ratio within an identified m/z range of the m/z ratio identified in Table 10. A first range for the product ion m/z ratio may be ±0.5. A second range for the product ion m/z ratio may be ±0.8. A third range for the product ion m/z ratio may be ±1.0. A first range for the precursor ion m/z ratio may be ±1.0; a second range for the precursor ion m/z ratio may be (±1.5). Thus, a composition may include a product ion having an m/z ratio that falls within at least one of the first range (±0.5), the second range (±0.8), or the third range (±1.0) of the product ion m/z ratio identified in Table 10, and characterized as having a precursor ion having an m/z ratio that falls within at least one of first range (±0.5), a second range (±1.0), or a third range (±1.0 of the precursor ion m/z ratio identified in Table 10.

Table 10 shows various parameters associated with the identification of the peptide and glycopeptides using LC and MRM-MS. The retention time (RT) represents the amount of time in minutes for the peptide to elute from the chromatography column. The collision energy represents the energy applied to the peptide for creating fragments (i.e., product ions) such as, for example, in the 2nd quadrupole of the triple quadrupole MS. The first precursor m/z represents a ratio value associated with an ionized form having a precursor charge for the peptide or glycopeptide. The precursor ion is associated with a first product ion having a m/z ratio that was formed from a collision and the second precursor ion is associated with a second product ion having a m/z ratio that was formed from a collision.

TABLE 10
Mass Spectrometry-Related Characteristics for the Peptide
Structures associated with Pancreatic Cancer

| PS-ID | Precursor | | Product | RT | Colli- |
|---|---|---|---|---|---|
| NO. | m/z | Precursor.charge | m/z | (min) | sion.Energy |
| PS-21 | 1131.1 | 3 | 366.1 | 26.2 | 28 |
| PS-5 | 1130.8 | 4 | 204.1 | 39.4 | 35 |
| PS-19 | 409.2 | 2 | 599.4 | 23.2 | 10 |
| PS-20 | 618.3 | 2 | 736.4 | 35.2 | 17 |
| PS-22 | 819.1 | 3 | 855.5 | 33.7 | 25 |
| PS-2 | 590.3 | 2 | 725.4 | 30.6 | 15 |
| PS-6 | 1087.8 | 3 | 366.1 | 10 | 30 |
| PS-17 | 1239.1 | 4 | 1314.2 | 38.8 | 25 |
| PS-3 | 1282.9 | 5 | 366.1 | 42.7 | 30 |
| PS-15 | 887.4 | 3 | 1360.6 | 13.1 | 30 |
| PS-1 | 1050.1 | 3 | 274.1 | 5 | 35 |
| PS-4 | 1173.6 | 6 | 366.1 | 13.2 | 29 |
| PS-7 | 1118.2 | 4 | 366.1 | 30.6 | 30 |
| PS-14 | 924.3 | 4 | 833.9 | 42.5 | 25 |
| PS-16 | 1147.8 | 5 | 366.1 | 41 | 25 |
| PS-12 | 1058.3 | 4 | 1284.7 | 31 | 20 |
| PS-18 | 772.4 | 2 | 680.3 | 22.7 | 22 |
| PS-13 | 1241.8 | 4 | 204.1 | 35.8 | 35 |
| PS-11 | 1142.2 | 4 | 366.1 | 35.9 | 20 |
| PS-10 | 561.8 | 2 | 244.2 | 9.5 | 25 |
| PS-9 | 1117.1 | 4 | 204.1 | 40.2 | 27 |
| PS-8 | 946.5 | 3 | 204.1 | 8.1 | 15 |

Table 11 defines the peptide sequences for SEQ ID NOS: 18, 21, 25, 28, 32, 51-57 from Table 8. Table 11 further identifies a corresponding protein SEQ ID NO. for each peptide sequence.

TABLE 11
Peptide SEQ ID NOS

| Pept | | |
|---|---|---|
| SEQ ID NO. | Peptide.sequence | Prot SEQ ID NO. |
| 51 | NEEYNK | 5 or 6 |
| 52 | DLLLPQPDLR | 41 |
| 53 | ADTHDEILEGLNFNLTEIPEAQIHEGFQELLR | 7 |
| 21 | NLFLNHSENATAK | 4 |
| 54 | VGQLQLSHNLSLVIL VPQNLK | 42 |
| 55 | DTFVNASR | 42 |
| 25 | YTGNASALFILPDQDK | 8 |
| 32 | EEQYNSTYR | 43 |
| 56 | LSLHRPALEDLLLGSEANLTCTLTGLR | 44 or 13 |
| 57 | VNHVTLSQPK | 45 |
| 58 | SVQEIQATFFYFTPNK | 5 or 6 |
| 59 | STGKPTLYNVSLVMSDTAGTCY | 15 |
| 60 | VLSNNSDANLELINTWVAK | 42 |
| 61 | ADTHDEILEGLNFNLTEIPEAQIHEGFQELLR | 7 |
| 62 | EEQFNSTFR | 46 |
| 63 | NCGVNCSGDVFTALIGEIASPNYPKPYPENSR | 47 |
| 64 | IITILEEEMNVSVCGLYTYGKPVPGHVTVSICR | 2 |
| 65 | SYTITGLQPGTDYK | 48 |
| 66 | AFLLTPR | 49 |
| 18 | DLATVYVDVLK | 1 |
| 28 | CGLVPVLAENYNK | 10 |
| 67 | TSESGELHGLTTEEEFVEGIYK | 50 |

Table 12 identifies the proteins of SEQ ID NOS: 1, 2, 4-8, 10, 13, 15, 41-50 from Table 8. Table 11 identifies a corresponding protein abbreviation and protein name for each of protein SEQ ID NOS: 1, 2, 4-8, 10, 13, 15, 41-50. Further, Table 12 identifies a corresponding Uniprot ID for each of protein SEQ ID NOS: 1, 2, 4-8, 10, 13, 15, 41-50.

TABLE 12
Protein SEQ ID NOS

| Prot | | | | |
|---|---|---|---|---|
| SEQ ID | Prot | Protein | Uniprot | Protein |
| NO. | Abbrev. | Name | ID No. | Sequence |
| 5 or 6 | AGP12 | Alpha-1- | P02763 or | MALSWVLTVLSLLPLLEAQIPLCANLVPVPITNATLDRIT |
| | | acid | P19652 (see | GKWFYIASAFRNEEYNKSVQEIQATFFYFTPNKTEDTIF |
| | | glycoprotein | Table 5) | LREYQTRQDQCIYNTTYLNVQRENGTISRYVGGQEHFA |
| | | 1 or 2 | | HLLILRDTKTYMLAFDVNDEKNWGLSVYADKPETTKE |
| | | | | QLGEFYEALDCLRIPKSDVVYTDWKKDKCEPLEKQHEK |
| | | | | ERKQEEGES |
| 41 | A2GL | Leucine-rich | P02750 | MSSWSRQRPKSPGGIQPHVSRTLFLLLLLAASAWGVTL |
| | | Alpha-2- | | SPKDCQVFRSDHGSSISCOPPAEIPGYLPADTVHLAVEFF |
| | | glycoprotein | | NLTHLPANLLQGASKLQELHLSSNGLESLSPEFLRPVPQ |
| | | | | LRVLDLTRNALTGLPPGLFQASATLDTLVLKENQLEVL |
| | | | | EVSWLHGLKALGHLDLSGNRLRKLPPGLLANFTLLRTL |
| | | | | DLGENQLETLPPDLLRGPLQLERLHLEGNKLQVLGKDL |
| | | | | LLPQPDLRYLFLNGNKLARVAAGAFQGLRQLDMLDLS |
| | | | | NNSLASVPEGLWASLGQPNWDMRDGFDISGNPWICDQ |
| | | | | NLSDLYRWLQAQKDKMFSQNDTRCAGPEAVKGQTLL |
| | | | | AVAKSQ |
| 7 | A1AT | Alpha-1- | P01009 | MPSSVSWGILLLAGLCCLVPVSLAEDPQGDAAQKTDTS |
| | | antitrypsin | | HHDQDHPTFNKITPNLAEFAFSLYRQLAHQSNSTNIFFSP |
| | | | | VSIATAFAMLSLGTKADTHDEILEGLNFNLTEIPEAQIHE |
| | | | | GFQELLRTLNQPDSQLQLTTGNGLFLSEGLKLVDKFLED |
| | | | | VKKLYHSEAFTVNFGDTEEAKKQINDYVEKGTQGKIVD |
| | | | | LVKELDRDTVFALVNYIFFKGKWERPFEVKDTEEEDFH |
| | | | | VDQVTTVKVPMMKRLGMFNIQHCKKLSSWVLLMKYL |
| | | | | GNATAIFFLPDEGKLQHLENELTHDIITKFLENEDRRSAS |
| | | | | LHLPKLSITGTYDLKSVLGQLGITKVFSNGADLSGVTEE |
| | | | | APLKLSKAVHKAVLTIDEKGTEAAGAMFLEAIPMSIPPE |
| | | | | VKFNKPFVFLMIEQNTKSPLFMGKVVNPTQK |
| 4 | HPT | Haptoglobin | P00738 | MSALGAVIALLLWGQLFAVDSGNDVTDIADDGCPKPPE |
| | | | | IAHGYVEHSVRYQCKNYYKLRTEGDGVYTLNDKKQWI |
| | | | | NKAVGDKLPECEADDGCPKPPEIAHGYVEHSVRYQCK |
| | | | | NYYKLRTEGDGVYTLNNEKQWINKAVGDKLPECEAVC |
| | | | | GKPKNPANPVQRILGGHLDAKGSFPWQAKMVSHHNLT |
| | | | | TGATLINEQWLLTTAKNLFLNHSENATAKDIAPTLTLYV |
| | | | | GKKQLVEIEKVVLHPNYSQVDIGLIKLKQKVSVNERVM |
| | | | | PICLPSKDYAEVGRVGYVSGWGRNANFKFTDHLKYVM |
| | | | | LPVADQDQCIRHYEGSTVPEKKTPKSPVGVQPILNEHTF |
| | | | | CAGMSKYQEDTCYGDAGSAFAVHDLEEDTWYATGILS |
| | | | | FDKSCAVAEYGVYVKVTSIQDWVOKTIAEN |
| 42 | IC1 | Plasma | P05155 | MASRLTLLTLLLLLLAGDRASSNPNATSSSSQDPESLQD |
| | | protease C1 | | RGEGKVATTVISKMLFVEPILEVSSLPTTNSTTNSATKIT |
| | | inhibitor | | ANTTDEPTTQPTTEPTTQPTIQPTQPTTQLPTDSPTQPTT |
| | | | | GSFCPGPVTLCSDLESHSTEAVLGDALVDFSLKLYHAFS |
| | | | | AMKKVETNMAFSPFSIASLLTQVLLGAGENTKTNLESIL |
| | | | | SYPKDFTCVHQALKGFTTKGVTSVSQIFHSPDLAIRDTF |
| | | | | VNASRTLYSSSPRVLSNNSDANLELINTWVAKNTNNKIS |
| | | | | RLLDSLPSDTRLVLLNAIYLSAKWKTTFDPKKTRMEPFH |
| | | | | FKNSVIKVPMMNSKKYPVAHFIDQTLKAKVGQLQLSH |
| | | | | NLSLVILVPQNLKHRLEDMEQALSPSVFKAIMEKLEMS |
| | | | | KFQPTLLTLPRIKVTTSQDMLSIMEKLEFFDFSYDLNLC |
| | | | | GLTEDPDLQVSAMQHQTVLELTETGVEAAAASAISVAR |
| | | | | TLLVFEVQQPFLFVLWDQQHKFPVFMGRVYDPRA |
| 8 | AACT | Alpha-1- | P01011 | MERMLPLLALGLLAAGFCPAVLCHPNSPLDEENLTQEN |
| | | antichymotrypsin | | QDRGTHVDLGLASANVDFAFSLYKQLVLKAPDKNVIFS |
| | | | | PLSISTALAFLSLGAHNTTLTEILKGLKFNLTETSEAEIHQ |
| | | | | SFQHLLRTLNQSSDELQLSMGNAMFVKEQLSLLDRFTE |
| | | | | DAKRLYGSEAFATDFQDSAAAKKLINDYVKNGTRGKIT |
| | | | | DLIKDLDSQTMMVLVNYIFFKAKWEMPFDPQDTHQSRF |
| | | | | YLSKKKWVMVPMMSLHHLTIPYFRDEELSCTVVELKY |
| | | | | TGNASALFILPDQDKMEEVEAMLLPETLKRWRDSLEFR |
| | | | | EIGELYLPKFSISRDYNLNDILLQLGIEEAFTSKADLSGIT |
| | | | | GARNLAVSQVVHKAVLDVFEEGTEASAATAVKITLLSA |
| | | | | LVETRTIVRFNRPFLMIIVPTDTQNIFFMSKVTNPKQA |
| 43 | IGG1 | Immunoglo | P01857 | ASTKGPSVFPLAPSSKSTSGGTAALGCLVKDYFPEPVTV |
| | | bulin heavy | | SWNSGALTSGVHTFPAVLQSSGLYSLSSVVTVPSSSLGT |
| | | constant | | QTYICNVNHKPSNTKVDKKVEPKSCDKTHTCPPCPAPE |
| | | gamma 1 | | LLGGPSVFLFPPKPKDTLMISRTPEVTCVVVDVSHEDPE |
| | | | | VKFNWYVDGVEVHNAKTKPREEQYNSTYRVVSVLTVL |
| | | | | HQDWLNGKEYKCKVSNKALPAPIEKTISKAKGQPREPQ |
| | | | | VYTLPPSRDELTKNQVSLTCLVKGFYPSDIAVEWESNG |
| | | | | QPENNYKTTPPVLDSDGSFFLYSKLTVDKSRWQQGNVF |
| | | | | SCSVMHEALHNHYTQKSLSLSPGK |
| 44 or 13 | IGA12 | Immunoglo | P01876 or | ASPTSPKVFPLSLCSTQPDGNVVIACLVQGFFPQEPLSVT |
| | | bulin heavy | P01877 (see | WSESGQGVTARNFPPSQDASGDLYTTSSQLTLPATQCL |
| | | constant | Table 5) | AGKSVTCHVKHYTNPSQDVTVPCPVPSTPPTPSPSTPPTP |
| | | alpha 1 or 2 | | SPSCCHPRLSLHRPALEDLLLGSEANLTCTLTGLRDASG |
| | | | | VTFTWTPSSGKSAVQGPPERDLCGCYSVSSVLPGCAEP |
| | | | | WNHGKTFTCTAAYPESKTPLTATLSKSGNTFRPEVHLLP |
| | | | | PPSEELALNELVTLTCLARGFSPKDVLVRWLQGSQELPR |
| | | | | EKYLTWASRQEPSQGTTTFAVTSILRVAAEDWKKGDTF |
| | | | | SCMVGHEALPLAFTQKTIDRLAGKPTHVNVSVVMAEV |
| | | | | DGTCY |
| 45 | B2M | Beta-2- | P61769 | MSRSVALAVLALLSLSGLEAIQRTPKIQVYSRHPAENGK |
| | | microglobulin | | SNFLNCYVSGFHPSDIEVDLLKNGERIEKVEHSDLSFSK |
| | | | | DWSFYLLYYTEFTPTEKDEYACRVNHVTLSQPKIVKWD |
| | | | | RDM |
| 5 or 6 | AGP12 | Alpha-1- | P02763 or | MALSWVLTVLSLLPLLEAQIPLCANLVPVPITNATLDRIT |
| | | acid | P19652 (see | GKWFYIASAFRNEEYNKSVQEIQATFFYFTPNKTEDTIF |
| | | glycoprotein | Table 5) | LREYQTRQDQCIYNTTYLNVQRENGTISRYVGGQEHFA |
| | | 1 or 2 | | HLLILRDTKTYMLAFDVNDEKNWGLSVYADKPETTKE |
| | | | | QLGEFYEALDCLRIPKSDVVYTDWKKDKCEPLEKQHEK |
| | | | | ERKQEEGES |
| 15 | IGM | Immunoglobulin | P01871 | GSASAPTLFPLVSCENSPSDTSSVAVGCLAQDFLPDSITF |
| | | heavy | | SWKYKNNSDISSTRGFPSVLRGGKYAATSQVLLPSKDV |
| | | constant mu | | MQGTDEHVVCKVQHPNGNKEKNVPLPVIAELPPKVSVF |
| | | | | VPPRDGFFGNPRKSKLICQATGFSPRQIQVSWLREGKQV |
| | | | | GSGVTTDQVQAEAKESGPTTYKVTSTLTIKESDWLGQS |
| | | | | MFTCRVDHRGLTFQQNASSMCVPDQDTAIRVFAIPPSFA |
| | | | | SIFLTKSTKLTCLVTDLTTYDSVTISWTRONGEAVKTHT |
| | | | | NISESHPNATFSAVGEASICEDDWNSGERFTCTVTHTDL |
| | | | | PSPLKQTISRPKGVALHRPDVYLLPPAREQLNLRESATIT |
| | | | | CLVTGFSPADVFVQWMQRGQPLSPEKYVTSAPMPEPQ |
| | | | | APGRYFAHSILTVSEEEWNTGETYTCVVAHEALPNRVT |
| | | | | ERTVDKSTGKPTLYNVSLVMSDTAGTCY |
| 46 | IGG2 | Immunoglobulin | P01859 | ASTKGPSVFPLAPCSRSTSESTAALGCLVKDYFPEPVTV |
| | | heavy | | SWNSGALTSGVHTFPAVLQSSGLYSLSSVVTVPSSNFGT |
| | | constant | | QTYTCNVDHKPSNTKVDKTVERKCCVECPPCPAPPVAG |
| | | gamma 2 | | PSVFLFPPKPKDTLMISRTPEVTCVVVDVSHEDPEVQFN |
| | | | | WYVDGVEVHNAKTKPREEQFNSTFRVVSVLTVVHQD |
| | | | | WLNGKEYKCKVSNKGLPAPIEKTISKTKGQPREPQVYT |
| | | | | LPPSREEMTKNQVSLTCLVKGFYPSDISVEWESNGQPEN |
| | | | | NYKTTPPMLDSDGSFFLYSKLTVDKSRWQQGNVFSCSV |
| | | | | MHEALHNHYTQKSLSLSPGK |
| 47 | C1S | Complement | P09871 | MWCIVLFSLLAWVYAEPTMYGEILSPNYPQAYPSEVEK |
| | | Cls | | SWDIEVPEGYGIHLYFTHLDIELSENCAYDSVQIISGDTE |
| | | subcomponent | | EGRLCGQRSSNNPHSPIVEEFQVPYNKLQVIFKSDFSNEE |
| | | | | RFTGFAAYYVATDINECTDFVDVPCSHFCNNFIGGYFCS |
| | | | | CPPEYFLHDDMKNCGVNCSGDVFTALIGEIASPNYPKPY |
| | | | | PENSRCEYQIRLEKGFQVVVTLRREDFDVEAADSAGNC |
| | | | | LDSLVFVAGDRQFGPYCGHGFPGPLNIETKSNALDIIFQT |
| | | | | DLTGQKKGWKLRYHGDPMPCPKEDTPNSVWEPAKAK |
| | | | | YVFRDVVQITCLDGFEVVEGRVGATSFYSTCQSNGKWS |
| | | | | NSKLKCQPVDCGIPESIENGKVEDPESTLFGSVIRYTCEE |
| | | | | PYYYMENGGGGEYHCAGNGSWVNEVLGPELPKCVPV |
| | | | | CGVPREPFEEKQRIIGGSDADIKNFPWQVFFDNPWAGG |
| | | | | ALINEYWVLTAAHVVEGNREPTMYVGSTSVQTSRLAK |
| | | | | SKMLTPEHVFIHPGWKLLEVPEGRTNFDNDIALVRLKD |
| | | | | PVKMGPTVSPICLPGTSSDYNLMDGDLGLISGWGRTEK |
| | | | | RDRAVRLKAARLPVAPLRKCKEVKVEKPTADAEAYVF |
| | | | | TPNMICAGGEKGMDSCKGDSGGAFAVQDPNDKTKFYA |
| | | | | AGLVSWGPQCGTYGLYTRVKNYVDWIMKTMQENSTP |
| | | | | RED |
| 2 | A2MG | Alpha-2- | P01023 | MGKNKLLHPSLVLLLLVLLPTDASVSGKPQYMVLVPSL |
| | | macroglobulin | | LHTETTEKGCVLLSYLNETVTVSASLESVRGNRSLFTDL |
| | | | | EAENDVLHCVAFAVPKSSSNEEVMFLTVQVKGPTQEFK |
| | | | | KRTTVMVKNEDSLVFVQTDKSIYKPGQTVKFRVVSMD |
| | | | | ENFHPLNELIPLVYIQDPKGNRIAQWQSFQLEGGLKQFS |
| | | | | FPLSSEPFQGSYKVVVQKKSGGRTEHPFTVEEFVLPKFE |
| | | | | VQVTVPKIITILEEEMNVSVCGLYTYGKPVPGHVTVSIC |
| | | | | RKYSDASDCHGEDSQAFCEKFSGQLNSHGCFYQQVKT |
| | | | | KVFQLKRKEYEMKLHTEAQIQEEGTVVELTGRQSSEITR |
| | | | | TITKLSFVKVDSHFRQGIPFFGQVRLVDGKGVPIPNKVIF |
| | | | | IRGNEANYYSNATTDEHGLVQFSINTTNVMGTSLTVRV |
| | | | | NYKDRSPCYGYQWVSEEHEEAHHTAYLVFSPSKSFVHL |
| | | | | EPMSHELPCGHTQTVQAHYILNGGTLLGLKKLSFYYLI |
| | | | | MAKGGIVRTGTHGLLVKQEDMKGHFSISIPVKSDIAPVA |
| | | | | RLLIYAVLPTGDVIGDSAKYDVENCLANKVDLSFSPSQS |
| | | | | LPASHAHLRVTAAPQSVCALRAVDQSVLLMKPDAELS |
| | | | | ASSVYNLLPEKDLTGFPGPLNDQDNEDCINRHNVYINGI |
| | | | | TYTPVSSTNEKDMYSFLEDMGLKAFTNSKIRKPKMCPQ |
| | | | | LQQYEMHGPEGLRVGFYESDVMGRGHARLVHVEEPHT |
| | | | | ETVRKYFPETWIWDLVVVNSAGVAEVGVTVPDTITEW |
| | | | | KAGAFCLSEDAGLGISSTASLRAFQPFFVELTMPYSVIR |
| | | | | GEAFTLKATVLNYLPKCIRVSVQLEASPAFLAVPVEKEQ |
| | | | | APHCICANGRQTVSWAVTPKSLGNVNFTVSAEALESQE |
| | | | | LCGTEVPSVPEHGRKDTVIKPLLVEPEGLEKETTENSLL |
| | | | | CPSGGEVSEELSLKLPPNVVEESARASVSVLGDILGSAM |
| | | | | QNTQNLLOMPYGCGEQNMVLFAPNIYVLDYLNETQQL |
| | | | | TPEIKSKAIGYLNTGYQRQLNYKHYDGSYSTFGERYGR |
| | | | | NQGNTWLTAFVLKTFAQARAYIFIDEAHITQALIWLSQR |
| | | | | QKDNGCFRSSGSLLNNAIKGGVEDEVTLSAYITIALLEIP |
| | | | | LTVTHPVVRNALFCLESAWKTAQEGDHGSHVYTKALL |
| | | | | AYAFALAGNQDKRKEVLKSLNEEAVKKDNSVHWERP |
| | | | | QKPKAPVGHFYEPQAPSAEVEMTSYVLLAYLTAQPAPT |
| | | | | SEDLTSATNIVKWITKQQNAQGGFSSTQDTVVALHALS |
| | | | | KYGAATFTRTGKAAQVTIQSSGTFSSKFQVDNNNRLLL |
| | | | | QQVSLPELPGEYSMKVTGEGCVYLQTSLKYNILPEKEEF |
| | | | | PFALGVQTLPQTCDEPKAHTSFQISLSVSYTGSRSASNM |
| | | | | AIVDVKMVSGFIPLKPTVKMLERSNHVSRTEVSSNHVLI |
| | | | | YLDKVSNQTLSLFFTVLQDVPVRDLKPAIVKVYDYYET |
| | | | | DEFAIAEYNAPCSKDLGNA |
| 48 | FINC | Fibronectin | P02751 | MLRGPGPGLLLLAVQCLGTAVPSTGASKSKRQAQQMV |
| | | | | QPQSPVAVSQSKPGCYDNGKHYQINQQWERTYLGNAL |
| | | | | VCTCYGGSRGFNCESKPEAEETCFDKYTGNTYRVGDTY |
| | | | | ERPKDSMIWDCTCIGAGRGRISCTIANRCHEGGQSYKIG |
| | | | | DTWRRPHETGGYMLECVCLGNGKGEWTCKPIAEKCFD |
| | | | | HAAGTSYVVGETWEKPYQGWMMVDCTCLGEGSGRIT |
| | | | | CTSRNRCNDQDTRTSYRIGDTWSKKDNRGNLLQCICTG |
| | | | | NGRGEWKCERHTSVQTTSSGSGPFTDVRAAVYQPQPHP |
| | | | | QPPPYGHCVTDSGVVYSVGMQWLKTQGNKQMLCTCL |
| | | | | GNGVSCQETAVTQTYGGNSNGEPCVLPFTYNGRTFYSC |
| | | | | TTEGRQDGHLWCSTTSNYEQDQKYSFCTDHTVLVQTR |
| | | | | GGNSNGALCHFPFLYNNHNYTDCTSEGRRDNMKWCGT |
| | | | | TONYDADQKFGFCPMAAHEEICTTNEGVMYRIGDQWD |
| | | | | KQHDMGHMMRCTCVGNGRGEWTCIAYSQLRDQCIVD |
| | | | | DITYNVNDTFHKRHEEGHMLNCTCFGQGRGRWKCDPV |
| | | | | DQCQDSETGTFYQIGDSWEKYVHGVRYQCYCYGRGIG |
| | | | | EWHCQPLQTYPSSSGPVEVFITETPSQPNSHPIQWNAPQ |
| | | | | PSHISKYILRWRPKNSVGRWKEATIPGHLNSYTIKGLKP |
| | | | | GVVYEGQLISIQQYGHQEVTRFDFTTTSTSTPVTSNTVT |
| | | | | GETTPFSPLVATSESVTEITASSFVVSWVSASDTVSGFRV |
| | | | | EYELSEEGDEPQYLDLPSTATSVNIPDLLPGRKYIVNVY |
| | | | | QISEDGEQSLILSTSQTTAPDAPPDTTVDQVDDTSIVVR |
| | | | | WSRPQAPITGYRIVYSPSVEGSSTELNLPETANSVTLSDL |
| | | | | QPGVQYNITIYAVEENQESTPVVIQQETTGTPRSDTVPSP |
| | | | | RDLQFVEVTDVKVTIMWTPPESAVTGYRVDVIPVNLPG |
| | | | | EHGQRLPISRNTFAEVTGLSPGVTYYFKVFAVSHGRESK |
| | | | | PLTAQQTTKLDAPTNLQFVNETDSTVLVRWTPPRAQIT |
| | | | | GYRLTVGLTRRGQPRQYNVGPSVSKYPLRNLQPASEYT |
| | | | | VSLVAIKGNQESPKATGVFTTLQPGSSIPPYNTEVTETTI |
| | | | | VITWTPAPRIGFKLGVRPSQGGEAPREVTSDSGSIVVSGL |
| | | | | TPGVEYVYTIQVLRDGQERDAPIVNKVVTPLSPPTNLHL |
| | | | | EANPDTGVLTVSWERSTTPDITGYRITTTPTNGQQGNSL |
| | | | | EEVVHADQSSCTFDNLSPGLEYNVSVYTVKDDKESVPIS |
| | | | | DTIIPEVPQLTDLSFVDITDSSIGLRWTPLNSSTIIGYRITV |
| | | | | VAAGEGIPIFEDFVDSSVGYYTVTGLEPGIDYDISVITLIN |
| | | | | GGESAPTTLTQQTAVPPPTDLRFTNIGPDTMRVTWAPPP |
| | | | | SIDLTNFLVRYSPVKNEEDVAELSISPSDNAVVLTNLLPG |
| | | | | TEYVVSVSSVYEQHESTPLRGRQKTGLDSPTGIDFSDIT |
| | | | | ANSFTVHWIAPRATITGYRIRHHPEHFSGRPREDRVPHS |
| | | | | RNSITLTNLTPGTEYVVSIVALNGREESPLLIGQQSTVSD |
| | | | | VPRDLEVVAATPTSLLISWDAPAVTVRYYRITYGETGG |
| | | | | NSPVQEFTVPGSKSTATISGLKPGVDYTITVYAVTGRGD |
| | | | | SPASSKPISINYRTEIDKPSQMQVTDVQDNSISVKWLPSS |
| | | | | SPVTGYRVTTTPKNGPGPTKTKTAGPDQTEMTIEGLQPT |
| | | | | VEYVVSVYAQNPSGESQPLVQTAVTNIDRPKGLAFTDV |
| | | | | DVDSIKIAWESPQGQVSRYRVTYSSPEDGIHELFPAPDG |
| | | | | EEDTAELQGLRPGSEYTVSVVALHDDMESQPLIGTQST |
| | | | | AIPAPTDLKFTQVTPTSLSAQWTPPNVQLTGYRVRVTPK |
| | | | | EKTGPMKEINLAPDSSSVVVSGLMVATKYEVSVYALKD |
| | | | | TLTSRPAQGVVTTLENVSPPRRARVTDATETTITISWRT |
| | | | | KTETITGFQVDAVPANGQTPIQRTIKPDVRSYTITGLQPG |
| | | | | TDYKIYLYTLNDNARSSPVVIDASTAIDAPSNLRFLATTP |
| | | | | NSLLVSWQPPRARITGYIIKYEKPGSPPREVVPRPRPGVT |
| | | | | EATITGLEPGTEYTIYVIALKNNQKSEPLIGRKKTDELPQ |
| | | | | LVTLPHPNLHGPEILDVPSTVQKTPFVTHPGYDTGNGIQ |
| | | | | LPGTSGQQPSVGQQMIFEEHGFRRTTPPTTATPIRHRPRP |
| | | | | YPPNVGEEIQIGHIPREDVDYHLYPHGPGLNPNASTGQE |
| | | | | ALSQTTISWAPFQDTSEYIISCHPVGTDEEPLQFRVPGTS |
| | | | | TSATLTGLTRGATYNVIVEALKDQQRHKVREEVVTVG |
| | | | | NSVNEGLNQPTDDSCFDPYTVSHYAVGDEWERMSESG |
| | | | | FKLLCQCLGFGSGHFRCDSSRWCHDNGVNYKIGEKWD |
| | | | | RQGENGQMMSCTCLGNGKGEFKCDPHEATCYDDGKT |
| | | | | YHVGEQWQKEYLGAICSCTCFGGQRGWRCDNCRRPGG |
| | | | | EPSPEGTTGQSYNQYSQRYHQRTNTNVNCPIECFMPLD |
| | | | | VQADREDSRE |
| 49 | APOM | Apolipoprotein | 095445 | MFHQIWAALLYFYGIILNSIYQCPEHSQLTTLGVDGKEF |
| | | M | | PEVHLGQWYFIAGAAPTKEELATFDPVDNIVENMAAGS |
| | | | | APMQLHLRATIRMKDGLCVPRKWIYHLTEGSTDLRTEG |
| | | | | RPDMKTELFSSSCPGGIMLNETGQGYQRFLLYNRSPHPP |
| | | | | EKCVEEFKSLTSCLDSKAFLLTPRNQEACELSNN |
| 1 | APOA1 | Apolipoprotein | P02647 | MKAAVLTLAVLFLTGSQARHFWQQDEPPQSPWDRVKD |
| | | A-I | | LATVYVDVLKDSGRDYVSQFEGSALGKQLNLKLLDNW |
| | | | | DSVTSTFSKLREQLGPVTQEFWDNLEKETEGLRQEMSK |
| | | | | DLEEVKAKVOPYLDDFQKKWQEEMELYRQKVEPLRAE |
| | | | | LQEGARQKLHELQEKLSPLGEEMRDRARAHVDALRTH |
| | | | | LAPYSDELRQRLAARLEALKENGGARLAEYHAKATEH |
| | | | | LSTLSEKAKPALEDLRQGLLPVLESFKVSFLSALEEYTK |
| | | | | KLNTQ |
| 10 | TRFE | Serotransferrin | P02787 | MRLAVGALLVCAVLGLCLAVPDKTVRWCAVSEHEAT |
| | | | | KCQSFRDHMKSVIPSDGPSVACVKKASYLDCIRAIAANE |
| | | | | ADAVTLDAGLVYDAYLAPNNLKPVVAEFYGSKEDPQT |
| | | | | FYYAVAVVKKDSGFQMNQLRGKKSCHTGLGRSAGWN |
| | | | | IPIGLLYCDLPEPRKPLEKAVANFFSGSCAPCADGTDFPQ |
| | | | | LCQLCPGCGCSTLNQYFGYSGAFKCLKDGAGDVAFVK |
| | | | | HSTIFENLANKADRDQYELLCLDNTRKPVDEYKDCHLA |
| | | | | QVPSHTVVARSMGGKEDLIWELLNQAQEHFGKDKSKE |
| | | | | FQLFSSPHGKDLLFKDSAHGFLKVPPRMDAKMYLGYE |
| | | | | YVTAIRNLREGTCPEAPTDECKPVKWCALSHHERLKCD |
| | | | | EWSVNSVGKIECVSAETTEDCIAKIMNGEADAMSLDGG |
| | | | | FVYIAGKCGLVPVLAENYNKSDNCEDTPEAGYFAIAVV |
| | | | | KKSASDLTWDNLKGKKSCHTAVGRTAGWNIPMGLLY |
| | | | | NKINHCRFDEFFSEGCAPGSKKDSSLCKLCMGSGLNLCE |
| | | | | PNNKEGYYGYTGAFRCLVEKGDVAFVKHQTVPQNTGG |
| | | | | KNPDPWAKNLNEKDYELLCLDGTRKPVEEYANCHLAR |
| | | | | APNHAVVTRKDKEACVHKILRQQQHLFGSNVTDCSGN |
| | | | | FCLFRSETKDLLFRDDTVCLAKLHDRNTYEKYLGEEYV |
| | | | | KAVGNLRKCSTSSLLEACTFRRP |
| 50 | TTR | Transthyretin | P02766 | MASHRLLLLCLAGLVFVSEAGPTGTGESKCPLMVKVLD |
| | | | | AVRGSPAINVAVHVFRKAADDTWEPFASGKTSESGELH |
| | | | | GLTTEEEFVEGIYKVEIDTKSYWKALGISPFHEHAEVVF |
| | | | | TANDSGPRRYTIAALLSPYSYSTTAVVTNPKE |

Table 13 identifies and defines the glycan structures included in Table 8. Table 13 identifies a coded representation of the composition for each glycan structure included in Table 8. As used herein, the 4-digit GL NO. is a designation that represents the number of hexoses, the number of HexNAcs, the number of Fucoses, and the number of Neuraminic Acids.

TABLE 13
Glycan Structure GL NOS: Composition

| Glycan | | |
|---|---|---|
| Structure | | Glycan |
| GL NO. | Glycan Symbol Structure | Composition |
| 5412 | 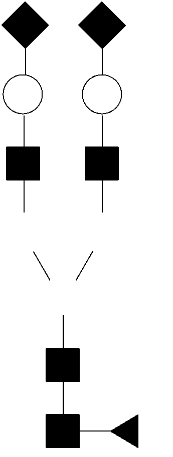 | Hex(5)HexNAc(4) Fuc(1)NeuAc(2) |
| 6512 | 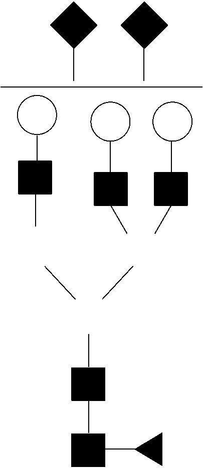 | Hex(6)HexNAc(5) Fuc(1)NeuAc(1) |
| 6502 or 6513 | 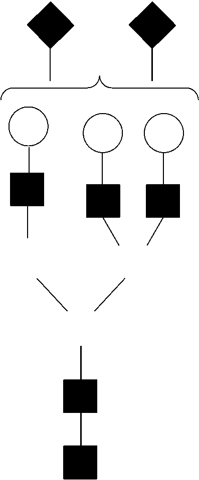 | Hex(6)HexNAc(5) Fuc(0)NeuAc(2); Hex(6)HexNAc(5) Fuc(1)NeuAc(3) |
| | or | |
| | 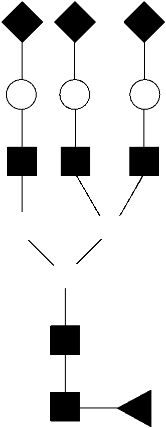 | |
| 5402 | 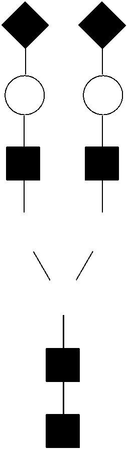 | Hex(5)HexNAc(4) Fuc(0)NeuAc(2) |
| 5412 | 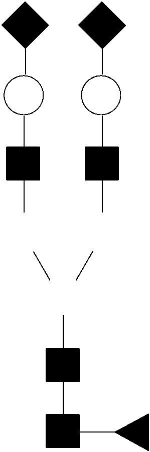 | Hex(5)HexNAc(4) Fuc(1)NeuAc(2) |
| 6512 | 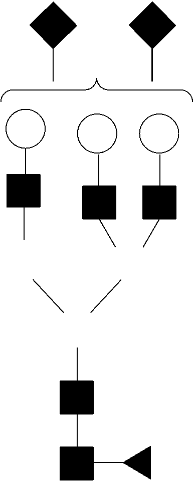 | Hex(6)HexNAc(5) Fuc(1)NeuAc(1) |
| 3510 | 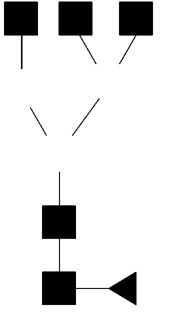 | Hex(3)HexNAc(5) Fuc(1)NeuAc(0) |
| | 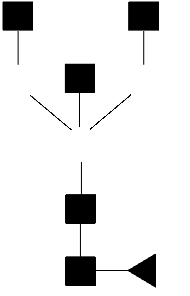 | |
| 3500 | 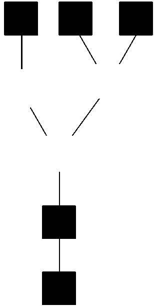 | Hex(3)HexNAc(5) Fuc(0)NeuAc(0) |
| | 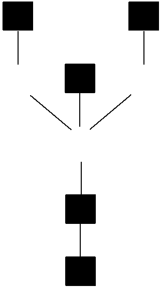 | |
| 7601 | 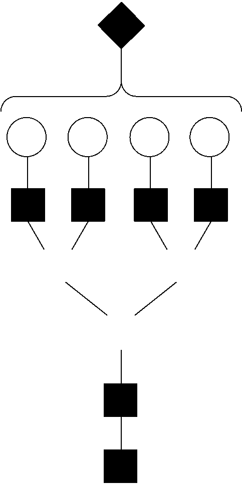 | Hex(7)HexNAc(6) Fuc(0)NeuAc(1) |
| 9200 | 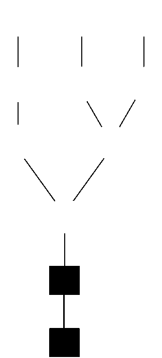 | Hex(9)HexNAc(2) Fuc(0)NeuAc(0) |
| 6503 | 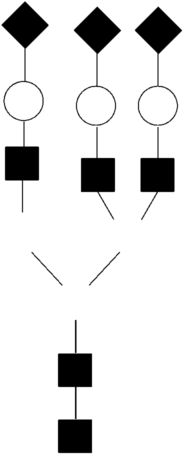 | Hex(6)HexNAc(5) Fuc(0)NeuAc(3) |
| 5402 | 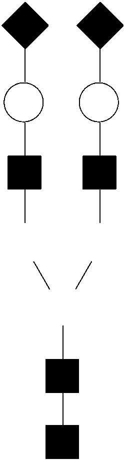 | Hex(5)HexNAc(4) Fuc(0)NeuAc(2) |
| 5200 | 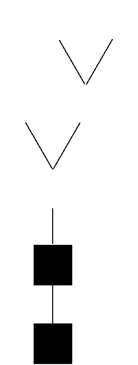 | Hex(5)HexNAc(2) Fuc(0)NeuAc(0) |
| 5401 | 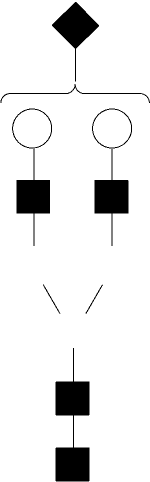 | Hex(5)HexNAc(4) Fuc(0)NeuAc(1) |

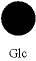

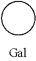

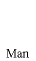

[symbol]

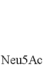

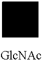

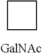

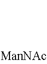

Table 13 illustrates the symbol structure and composition of detected glycan moieties that correspond to glycopeptides of Table 8, based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. For reference, N-linked glycans have a glycan attached to the amino acid asparagine and O-linked glycans have a glycan attached to either a serine or a threonine. All of the glycans in Table 13 represent N-linked glycans.

For some entries, there are two symbol structures provided for one Glycan Structure GL NO such as, for example, Glycan Structure GL NO 3510 in Table 13. Thus, the identity of a peptide that references a Glycan Structure GL NO that has two symbol structures could be one of two possibilities based on the MRM of the LC-MS analysis.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

In some instances, a bracket symbol is used as part of the Symbol Structure (e.g., 4310) to indicate that the precise bonding linkage is not exactly known, but that the linking line segment is attached to one of the plurality of adjacent carbohydrates immediately adjacent to the bracket.

The identity of the various monosaccharides is illustrated by the Legend section located at the end of Table 13. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating a PC disease state. A transition includes a precursor ion and at least one product ion grouping. As reviewed herein, the peptide structures in Table 8, as well as their corresponding precursor ion and product ion groupings (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Table 10 or an m/z ratio within an identified m/z ratio as provided in Table 10. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### XI. Group II Representative Experimental Results

### XI.A. Subject Sample Models

To assess the association of individual peptide structures (biomarkers) with pancreatic cancer, three differential expression analyses (DEAs) were run on three different subject cohorts, adjusting for age and sex.

Table 14 below identifies the fold changes, FDRs, and p-values as determined by the differential expression analysis (DEA) performed for the markers. These DEA results yielded 25 markers that satisfied FDR 1012 and concordance (AUC)≥0.7.

Model Analysis: The subject cohort for the first differential expression analysis included 290 subjects diagnosed with pancreatic cancer and 194 healthy control subjects. The samples for the model were obtained from Precision for Medicine (healthy controls) and both Indivumed and iSpecimen for cancer samples. The fold change, FDR, and p-value information relevant to the markers for the model can be identified by referencing the information provided in Table 14.

TABLE 14
Differential Expression Analysis (DEA) for Group II

| | | Diff. expr. | Diff. expr. | Diff. expr. |
|---|---|---|---|---|
| PS-ID | | (pancr./healthy | (pancr./healthy | (pancr./ |
| NO. | PS-NAME | fold change) | p-value) | healthy FDR) |
| PS-21 | TRFE_432_5401 | 0.628 | 3.52E-25 | 1.05E-22 |
| PS-5 | IC1_352_5402 | 1.767 | 1.55E-20 | 3.07E-18 |
| PS-19 | QUANTPEP.APOM_AFLLTPR | 0.763 | 1.08E-19 | 1.60E-17 |
| PS-20 | QUANTPEP.APOA1_DLATVYVDVLK | 0.697 | 6.52E-19 | 7.77E-17 |
| PS-22 | QUANTPEP.TTR_TSESGELHGLTTEEEFV | 0.784 | 1.94E-18 | 1.93E-16 |
| | EGIYK | | | |
| PS-2 | QUANTPEP.A2GL_DLLLPQPDLR | 1.532 | 8.65E-18 | 6.44E-16 |
| PS-6 | IC1_238_5412 | 1.95 | 1.14E-17 | 7.52E-16 |
| PS-17 | A2MG_247_5200 | 0.603 | 1.37E-17 | 8.19E-16 |
| PS-3 | A1AT_107_6512 | 2.044 | 8.05E-17 | 3.69E-15 |
| PS-15 | IGG2_297_3500 | 0.402 | 2.94E-16 | 1.10E-14 |
| PS-1 | AGP12_56_5412 | 1.591 | 4.33E-14 | 1.08E-12 |
| PS-4 | HPT_207_121015 | 2.151 | 3.51E-11 | 4.64E-10 |
| PS-7 | AACT_271_6512 | 1.613 | 4.21E-11 | 5.45E-10 |
| PS-14 | A1AT_107_nonglycosylated | 0.345 | 2.89E-10 | 3.07E-09 |
| PS-16 | C1S_174_5402 | 0.784 | 9.99E-10 | 8.92E-09 |
| PS-12 | IGM_439_9200 | 2.83 | 1.13E-09 | 9.60E-09 |
| PS-18 | QUANTPEP.FINC_SYTITGLQPGTDYK | 0.636 | 8.35E-09 | 6.46E-08 |
| PS-13 | IC1_253_6503 | 0.764 | 1.66E-07 | 9.72E-07 |
| PS-11 | AGP12_72_7601 | 1.309 | 8.19E-07 | 4.04E-06 |
| PS-10 | QUANTPEP.B2M_VNHVTLSQPK | 1.176 | 5.45E-06 | 2.21E-05 |
| PS-9 | IGA12_144_3500 | 1.305 | 5.54E-05 | 1.77E-04 |
| PS-8 | IGG1_297_3510 | 1.231 | 1.23E-04 | 3.62E-04 |

### XI.B. Training a Binary Classification Model

A full panel of biomarkers were included in training a binary classification model for diagnosing pancreatic cancer status. For the various models discussed herein, the total number of subjects was split into 70% training (n=159) and 30% testing (n=67). For the training set, repeated, 10-fold cross-validation was used to select optimal hyperparameters for LASSO, and then these hyperparameters were used on the entire training set develop one predictive logistic regression model. This model was then blindly used to predict pancreatic cancer status in the test set. Overall, 22 markers were left with non-zero weights after LASSO shrinkage for the associated model. These 22 markers are identified in Table 14 above.

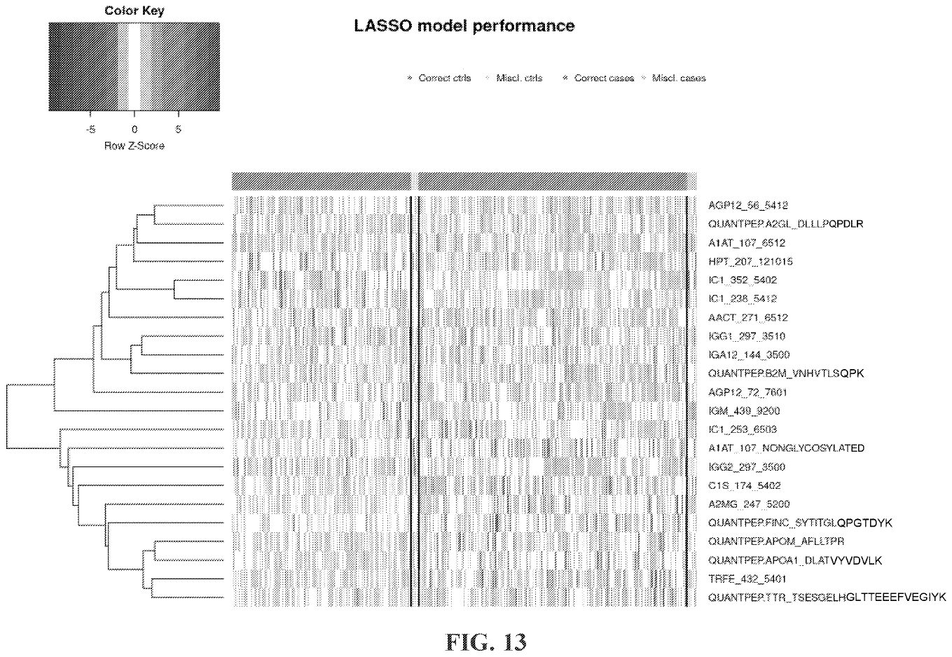

FIGS. 13-16 are example explanatory illustrations that correspond to the model. For example, FIG. 13 is a marker-wise hierarchically-clustered heat map comparing z-score values of biomarker expression levels for retained biomarkers in the model across patent data set, in accordance with one or more embodiments. Columns represent patient samples, grouped by healthy control and pancreatic cancer status, and whether the model correctly or incorrectly classified a specific patient sample.

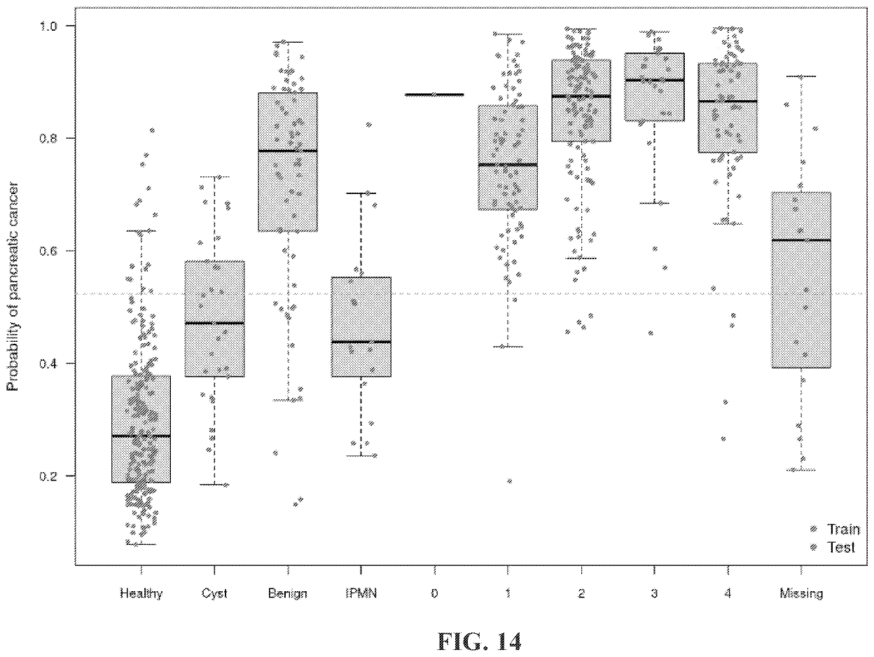

FIG. 14 is a probability dotplot illustrating probabilities of pancreatic cancer across training and test data across various patient sample entities, including pancreatic cancer stage, in accordance with one or more embodiments.

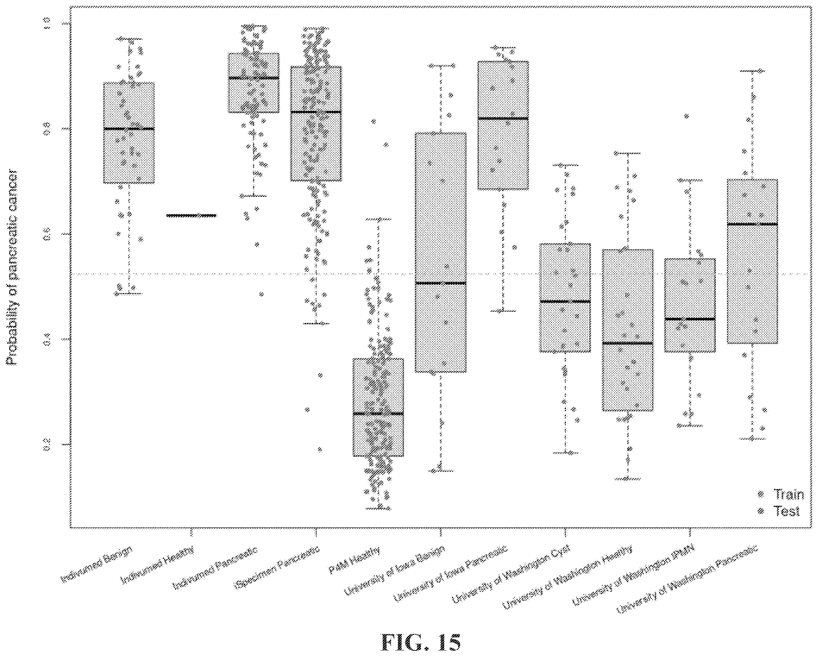

FIG. 15 is a probability dotplot illustrating probabilities of pancreatic cancer across training and test data across various sample sources and entities, in accordance with one or more embodiments.

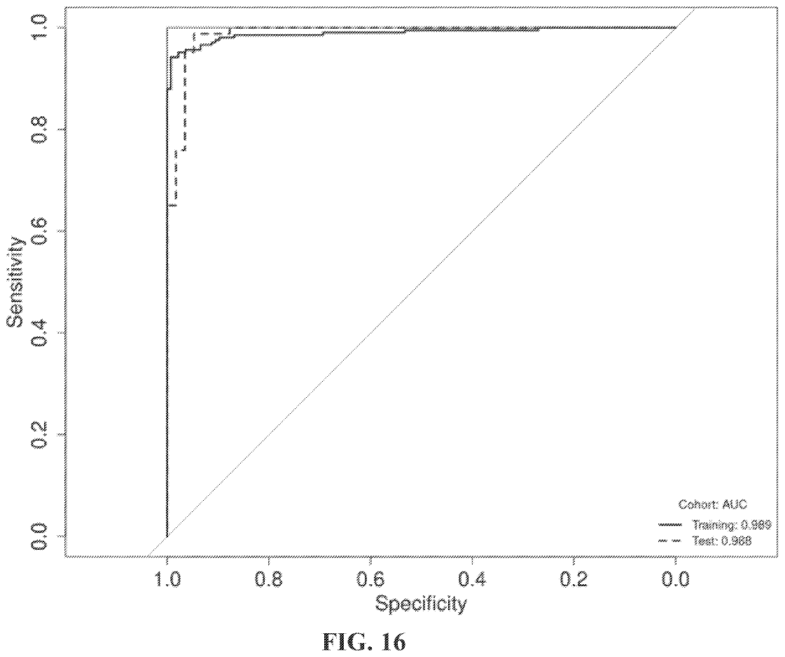

FIG. 16 is an example plot of a receiver operating characteristic (ROC) curve for the model for the training set and testing set in accordance with one or more embodiments. The plot illustrates specificity versus sensitivity. The area under the curve (AUC) for the training set was found to be 0.989 and the AUC for the testing set was found to be 0.988.

### XII. Recitation of Embodiments

### Embodiment 1

A method for diagnosing a subject with respect to a pancreatic cancer (PC) disease state, the method comprising:
receiving peptide structure data corresponding to a biological sample obtained from the subject;
analyzing the peptide structure data using a supervised machine learning model to generate a disease indicator that indicates whether the biological sample evidences a PC disease state based on at least 3 peptide structures selected from a group of peptide structures identified in Table 1,
   wherein the group of peptide structures in Table 1 is associated with the PC disease state; and
   wherein the group of peptide structures is listed in Table 1 with respect to relative significance to the disease indicator; and
generating a diagnosis output based on the disease indicator.

### Embodiment 2

The method of Embodiment 1, wherein the disease indicator comprises a score.

### Embodiment 3

The method of Embodiment 2, wherein generating the diagnosis output comprises:
determining that the score falls above a selected threshold; and
generating the diagnosis output based on the score falling above the selected threshold, wherein the diagnosis output includes a positive diagnosis for the PC disease state.

### Embodiment 4

The method of Embodiment 2, wherein generating the diagnosis output comprises:
determining that the score falls below a selected threshold; and
generating the diagnosis output based on the score falling below the selected threshold, wherein the diagnosis output includes a negative diagnosis for the PC disease state.

### Embodiment 5

The method of Embodiment 3 or Embodiment 4, wherein the score comprises a probability score and the selected threshold is 0.5.

### Embodiment 6

The method of Embodiment 3 or Embodiment 4, wherein the selected threshold falls within a range between 0.4 and 0.6.

### Embodiment 7

The method of any one of Embodiments 1-6, wherein analyzing the peptide structure data comprises:
analyzing the peptide structure data using a binary classification model.

### Embodiment 8

The method of any one of Embodiments 1-7, wherein the at least one peptide structure comprises a glycopeptide structure defined by a peptide sequence and a glycan structure linked to the peptide sequence at a linking site of the peptide sequence, as identified in Table 1, with the peptide sequence being one of SEQ ID NOS: 18-40 as defined in Table 1.

### Embodiment 9

The method of any one of Embodiments 1-8, further comprising:
training the supervised machine learning model using training data,
wherein the training data comprises a plurality of peptide structure profiles for a plurality of subjects and a plurality of subject diagnoses for the plurality of subjects.

### Embodiment 10

The method of Embodiment 9, wherein the plurality of subject diagnoses includes a positive diagnosis for any subject of the plurality of subjects determined to have the PC disease state and a negative diagnosis for any subject of the plurality of subjects determined not to have the PC disease state.

### Embodiment 11

The method of any one of Embodiments 9-10, further comprising:
performing a differential expression analysis using initial training data to compare a first portion of the plurality of subjects diagnosed with the positive diagnosis for the PC disease state versus a second portion of the plurality of subjects diagnosed with the negative diagnosis for the PC disease state; and
identifying a training group of peptide structures based on the differential expression analysis for use as prognostic markers for the PC disease state; and
forming the training data based on the training group of peptide structures identified.

### Embodiment 12

The method of Embodiment 11, wherein training the supervised machine learning model comprises reducing the training group of peptide structures to a final group of peptide structures identified in Table 2.

### Embodiment 13

The method of any one of Embodiments 10-12, wherein the negative diagnosis for the PC disease state indicates a non-pancreatic cancer (PC) state comprising at least one of a healthy state, a benign pancreatitis state, or a control state.

### Embodiment 14

The method of any one of Embodiments 1-13, wherein the supervised machine learning model comprises a logistic regression model.

### Embodiment 15

The method of any one of Embodiments 1-14, wherein the at least 3 peptide structures are included in Table 2, wherein Table 2 identifies a final group of peptide structures that is a subset of the group of peptide structures identified in Table 1.

### Embodiment 16

The method of any one of Embodiments 1-15, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

### Embodiment 17

The method of any one of Embodiments 1-16, wherein the peptide structure data is generated using multiple reaction monitoring mass spectrometry (MRM-MS).

### Embodiment 18

The method of any one of Embodiments 1-17, further comprising:
creating a sample from the biological sample; and
preparing the sample using reduction, alkylation, and enzymatic digestion to form a prepared sample that includes a set of peptide structures.

### Embodiment 19

The method of Embodiment 18, further comprising:
generating the peptide structure data from the prepared sample using multiple reaction monitoring mass spectrometry (MRM-MS).

### Embodiment 20

The method of any one of Embodiments 1-19, wherein generating the diagnosis output comprises:
generating a report identifying that the biological sample evidences the PC disease state.

### Embodiment 21

The method of any one of Embodiments 1-20, further comprising:
generating a treatment output based on at least one of the diagnosis output or the disease indicator.

### Embodiment 22

The method of Embodiment 20, wherein the treatment output comprises at least one of an identification of a treatment to treat the subject or a treatment plan.

### Embodiment 23

The method of Embodiment 21, wherein the treatment comprises at least one of radiation therapy, chemoradiotherapy, surgery, or a targeted drug therapy.

### Embodiment 24

A method of training a model to diagnose a subject with respect to a pancreatic cancer (PC) disease state, the method comprising:
receiving quantification data for a panel of peptide structures for a plurality of subjects,
   wherein the plurality of subjects includes a first portion diagnosed with a negative diagnosis of a PC disease state and a second portion diagnosed with a positive diagnosis of the PC disease state;
   wherein the quantification data comprises a plurality of peptide structure profiles for the plurality of subjects; and
training a machine learning model using the quantification data to diagnose a biological sample with respect to the PC disease state using a group of peptide structures associated with the PC disease state,
   wherein the group of peptide structures is identified in Table 1; and
   wherein the group of peptide structures is listed in Table 1 with respect to relative significance to diagnosing the biological sample.

### Embodiment 25

The method of Embodiment 24, wherein the machine learning model comprises a logistic regression model.

### Embodiment 26

The method of Embodiment 25, wherein the logistic regression model comprises a LASSO regression model.

### Embodiment 27

The method of any one of Embodiments 23-26, wherein training the machine learning model comprises:
training the machine learning using a portion of the quantification data corresponding to a training group of peptide structures included in the plurality of peptide structures.

### Embodiment 28

The method of Embodiment 27, further comprising:
performing a differential expression analysis using the quantification data for the plurality of subjects.

### Embodiment 29

The method of Embodiment 28, further comprising:
identifying the training group of peptide structures based on the differential expression analysis, wherein the training group of peptide structures is a subset of the plurality of peptide structures that has been determined to be relevant to diagnosing the PC disease state.

### Embodiment 30

The method of Embodiment 29, wherein training the machine learning model comprises reducing the training group of peptide structures to a final group of peptide structures identified in Table 2.

### Embodiment 31

The method of any one of Embodiments 24-30, wherein the negative diagnosis for the PC state indicates a non-pancreatic cancer (PC) state comprising at least one of a healthy state, a benign pancreatitis state, or a control state.

### Embodiment 32

The method of any one of Embodiments 24-31, wherein the quantification data for the panel of peptide structures for the plurality of subjects diagnosed with the plurality of PC disease states comprises at least one of an abundance, a relative abundance, a normalized abundance, a relative quantity, an adjusted quantity, a normalized quantity, a relative concentration, an adjusted concentration, or a normalized concentration.

### Embodiment 33

A method of monitoring a subject for a pancreatic cancer (PC) disease state, the method comprising:
receiving first peptide structure data for a first biological sample obtained from a subject at a first timepoint;
analyzing the first peptide structure data using a supervised machine learning model to generate a first disease indicator based on at least 3 peptide structures selected from a group of peptide structures identified in Table 1, wherein the group of peptide structures in Table 1 comprises a group of peptide structures associated with a PC disease state;
receiving second peptide structure data of a second biological sample obtained from the subject at a second timepoint;
analyzing the second peptide structure data using the supervised machine learning model to generate a second disease indicator based on the at least 3 peptide structures selected from the group of peptide structures identified in Table 1; and
generating a diagnosis output based on the first disease indicator and the second disease indicator.

### Embodiment 34

The method of Embodiment 33, wherein the at least 3 peptide structures are included in Table 2, wherein Table 2 identifies a final group of peptide structures that is a subset of the group of peptide structures in Table 1.

### Embodiment 35

The method of Embodiment 33 or Embodiment 34, wherein generating the diagnosis output comprises:
comparing the second disease indicator to the first disease indicator.

### Embodiment 36

The method of any one of Embodiments 33-35, wherein the first disease indicator indicates that the first biological sample evidences a negative diagnosis for the PC disease state and the second biological sample evidences a positive diagnosis for the PC disease state.

### Embodiment 37

The method of any one of Embodiments 33-36, wherein the diagnosis output identifies whether a non-PC disease state has progressed to the PC disease state, wherein the non-PC disease state includes either a healthy state or a benign pancreatitis state.

### Embodiment 38

The method of any one of Embodiments 33-37, wherein the supervised machine learning model comprises a logistic regression model.

### Embodiment 39

A composition comprising at least one of peptide structures PS-1 to PS-38 identified in Table 1.

### Embodiment 40

A composition comprising at least one of peptide structures PS-1 to PS-5, PS-8, PS-9, PS-12 to PS-15, PS-17, PS-20, PS-26, and PS-33 to PS-38 identified in Table 2.

### Embodiment 41

A composition comprising a peptide structure or a product ion, wherein:
the peptide structure or the product ion comprises an amino acid sequence having at least 90% sequence identity to any one of SEQ ID NOS: 18-40, corresponding to peptide structures PS-1 to PS-38 in Table 1; and
the product ion is selected as one from a group consisting of product ions identified in Table 3 including product ions falling within an identified m/z range.

### Embodiment 42

A composition comprising a glycopeptide structure selected as one from a group consisting of peptide structures PS-1 to PS-38 identified in Table 1, wherein: the glycopeptide structure comprises:
an amino acid peptide sequence identified in Table 4 as corresponding to the glycopeptide structure; and
a glycan structure identified in Table 6 as corresponding to the glycopeptide structure in which the glycan structure is linked to a residue of the amino acid peptide sequence at a corresponding position identified in Table 1; and
   wherein the glycan structure has a glycan composition.

### Embodiment 43

The composition of Embodiment 42, wherein the glycan composition is identified in Table 6.

### Embodiment 44

The composition of Embodiment 42 or Embodiment 43, wherein:
the glycopeptide structure has a precursor ion having a charge identified in Table 3 as corresponding to the glycopeptide structure.

### Embodiment 45

The composition of any one of Embodiments 42-44, wherein:
the glycopeptide structure has a precursor ion with an m/z ratio within ±1.5 of the m/z ratio listed for the precursor ion in Table 3 as corresponding to the glycopeptide structure.

### Embodiment 46

The composition of any one of Embodiments 42-44, wherein:
the glycopeptide structure has a precursor ion with an m/z ratio within ±1.0 of the m/z ratio listed for the precursor ion in Table 3 as corresponding to the glycopeptide structure.

### Embodiment 47

The composition of any one of Embodiments 42-44, wherein:
the glycopeptide structure has a precursor ion with an m/z ratio within ±0.5 of the m/z ratio listed for the precursor ion in Table 3 as corresponding to the glycopeptide structure.

### Embodiment 48

The composition of any one of Embodiments 42-47, wherein:
the glycopeptide structure has a product ion with an m/z ratio within ±1.0 of the m/z ratio listed for the product ion in Table 3 as corresponding to the glycopeptide structure.

### Embodiment 49

The composition of any one of Embodiments 42-47, wherein:
the glycopeptide structure has a product ion with an m/z ratio within ±0.8 of the m/z ratio listed for the product ion in Table 3 as corresponding to the glycopeptide structure.

### Embodiment 50

The composition of any one of Embodiments 42-47, wherein:
the glycopeptide structure has a product ion with an m/z ratio within ±0.5 of the m/z ratio listed for the product ion in Table 3 as corresponding to the glycopeptide structure.

### Embodiment 51

The composition of any one of Embodiments 42-50, wherein the glycopeptide structure has a monoisotopic mass identified in Table 1 as corresponding to the glycopeptide structure.

### Embodiment 52

A composition comprising a peptide structure selected as one from a plurality of peptide structures identified in Table 1, wherein:
the peptide structure has a monoisotopic mass identified as corresponding to the peptide structure in Table 1; and
the peptide structure comprises the amino acid sequence of SEQ ID NOs: 18-40 identified in Table 1 as corresponding to the peptide structure.

### Embodiment 53

The composition of Embodiment 52, wherein:
the peptide structure has a precursor ion having a charge identified in Table 3 as corresponding to the peptide structure.

### Embodiment 54

The composition of Embodiment 52 or Embodiment 53, wherein:
the peptide structure has a precursor ion with an m/z ratio within ±1.5 of the m/z ratio listed for the precursor ion in Table 3 as corresponding to the peptide structure.

### Embodiment 55

The composition of Embodiment 52 or Embodiment 53, wherein:
the peptide structure has a precursor ion with an m/z ratio within ±1.0 of the m/z ratio listed for the precursor ion in Table 3 as corresponding to the peptide structure.

### Embodiment 56

The composition of Embodiment 52 or Embodiment 53, wherein:
the peptide structure has a precursor ion with an m/z ratio within ±0.5 of the m/z ratio listed for the precursor ion in Table 3 as corresponding to the peptide structure.

### Embodiment 57

The composition of any one of Embodiments 52-56, wherein:
the peptide structure has a product ion with an m/z ratio within ±1.0 of the m/z ratio listed for the product ion in Table 3 as corresponding to the peptide structure.

### Embodiment 58

The composition of any one of Embodiments 52-56, wherein:
the peptide structure has a product ion with an m/z ratio within ±0.8 of the m/z ratio listed for the product ion in Table 3 as corresponding to the peptide structure.

### Embodiment 59

The composition of any one of Embodiments 52-56, wherein:
the peptide structure has a product ion with an m/z ratio within ±0.5 of the m/z ratio listed for the product ion in Table 3 as corresponding to the peptide structure.

### Embodiment 60

A kit comprising at least one agent for quantifying at least one peptide structure identified in Table 1 to carry out part or all of the method of any one of Embodiments 1-38.

### Embodiment 61

A kit comprising at least one agent for quantifying at least one peptide structure identified in Table 2 to carry out part or all of the method of any one of Embodiments 1-38.

### Embodiment 62

A kit comprising at least one of a glycopeptide standard, a buffer, or a set of peptide sequences to carry out part or all of the method of any one of Embodiments 1-38, a peptide sequence of the set of peptide sequences identified by a corresponding one of SEQ ID NOS: 18-40, defined in Table 1.

### Embodiment 63

A system comprising:
one or more data processors; and
a non-transitory computer readable storage medium containing instructions which, when executed on the one or more data processors, cause the one or more data processors to perform part or all of any one of Embodiments 1-38.

### Embodiment 64

A computer-program product tangibly embodied in a non-transitory machine-readable storage medium, including instructions configured to cause one or more data processors to perform part or all of any one of Embodiments 1-38.

### Embodiment 65

A composition comprising at least one of peptide structures PS-1 to PS-8, PS-10 to PS-14, PS-16 to PS-19, PS-21 to PS-25, PS-28 to PS-29, PS-31 to PS-34, PS-36 to PS-38 identified in Table 1.

### Embodiment 66

A composition comprising a peptide structure or a product ion, wherein:
the peptide structure or the product ion comprises an amino acid sequence having at least 90% sequence identity to any one of SEQ ID NOS: 18-23, 25-28, 30-32, 35-36, and 38-40; and
the product ion is selected as one from a group consisting of product ions identified in Table 3 including product ions falling within an identified m/z range.

### Embodiment 67

A composition comprising a glycopeptide structure selected as one from a group consisting of peptide structures PS-1 to PS-8, PS-10 to PS-14, PS-16 to PS-19, PS-21 to PS-25, PS-28 to PS-29, PS-31 to PS-34, PS-36 to PS-38 identified in Table 1, wherein:
the glycopeptide structure comprises:
   an amino acid peptide sequence identified in Table 4 as corresponding to the glycopeptide structure; and
   a glycan structure identified in Table 6 as corresponding to the glycopeptide structure in which the glycan structure is linked to a residue of the amino acid peptide sequence at a corresponding position identified in Table 1; and
      wherein the glycan structure has a glycan composition.

### Embodiment 68

The composition of Embodiment 67, wherein the glycan composition is identified in Table 6.

### Embodiment 69

The composition of Embodiment 67 or Embodiment 68, wherein:
the glycopeptide structure has a precursor ion having a charge identified in Table 3 as corresponding to the glycopeptide structure.

### Embodiment 70

The composition of any one of Embodiments 67-69, wherein:
the glycopeptide structure has a precursor ion with an m/z ratio within ±1.5 of the m/z ratio listed for the precursor ion in Table 3 as corresponding to the glycopeptide structure.

### Embodiment 71

The composition of any one of Embodiments 67-69, wherein:
the glycopeptide structure has a precursor ion with an m/z ratio within ±1.0 of the m/z ratio listed for the precursor ion in Table 3 as corresponding to the glycopeptide structure.

### Embodiment 72

The composition of any one of Embodiments 67-69, wherein:
the glycopeptide structure has a precursor ion with an m/z ratio within ±0.5 of the m/z ratio listed for the precursor ion in Table 3 as corresponding to the glycopeptide structure.

### Embodiment 73

The composition of any one of Embodiments 67-72, wherein:
the glycopeptide structure has a product ion with an m/z ratio within ±1.0 of the m/z ratio listed for the product ion in Table 3 as corresponding to the glycopeptide structure.

### Embodiment 74

The composition of any one of Embodiments 67-72, wherein:
the glycopeptide structure has a product ion with an m/z ratio within ±0.8 of the m/z ratio listed for the product ion in Table 3 as corresponding to the glycopeptide structure.

### Embodiment 75

The composition of any one of Embodiments 67-72, wherein:
the glycopeptide structure has a product ion with an m/z ratio within ±0.5 of the m/z ratio listed for the product ion in Table 3 as corresponding to the glycopeptide structure.

### Embodiment 76

The composition of any one of Embodiments 67-75, wherein the glycopeptide structure has a monoisotopic mass identified in Table 1 as corresponding to the glycopeptide structure.

### Embodiment 77

A composition comprising a peptide structure selected as one of PS-1 to PS-8, PS-10 to PS-14, PS-16 to PS-19, PS-21 to PS-25, PS-28 to PS-29, PS-31 to PS-34, PS-36 to PS-38 peptide structures identified in Table 1, wherein:
the peptide structure has a monoisotopic mass identified as corresponding to the peptide structure in Table 1; and
the peptide structure comprises the amino acid sequence of SEQ ID NOs: SEQ ID NOS: 18-23, 25-28, 30-32, 35-36, and 38-40 identified in Table 1 as corresponding to the peptide structure.

### Embodiment 78

The composition of Embodiment 77, wherein:
the peptide structure has a precursor ion having a charge identified in Table 3 as corresponding to the peptide structure.

### Embodiment 79

The composition of Embodiment 77 or Embodiment 78, wherein:
the peptide structure has a precursor ion with an m/z ratio within ±1.5 of the m/z ratio listed for the precursor ion in Table 3 as corresponding to the peptide structure.

### Embodiment 80

The composition of Embodiment 77 or Embodiment 78, wherein:
the peptide structure has a precursor ion with an m/z ratio within ±1.0 of the m/z ratio listed for the precursor ion in Table 3 as corresponding to the peptide structure.

### Embodiment 81

The composition of Embodiment 77 or Embodiment 78, wherein:
the peptide structure has a precursor ion with an m/z ratio within ±0.5 of the m/z ratio listed for the precursor ion in Table 3 as corresponding to the peptide structure.

### Embodiment 82

The composition of any one of Embodiments 77-81, wherein:
the peptide structure has a product ion with an m/z ratio within ±1.0 of the m/z ratio listed for the product ion in Table 3 as corresponding to the peptide structure.

### Embodiment 83

The composition of any one of Embodiments 77-81, wherein:
the peptide structure has a product ion with an m/z ratio within ±0.8 of the m/z ratio listed for the product ion in Table 3 as corresponding to the peptide structure.

### Embodiment 84

The composition of any one of Embodiments 77-81, wherein:
the peptide structure has a product ion with an m/z ratio within ±0.5 of the m/z ratio listed for the product ion in Table 3 as corresponding to the peptide structure.

### Embodiment 85

A method for diagnosing a subject with respect to a pancreatic cancer (PC) disease state, the method comprising:
receiving peptide structure data corresponding to a biological sample obtained from the subject;
analyzing the peptide structure data using a supervised machine learning model to generate a disease indicator that indicates whether the biological sample evidences a PC disease state based on at least 3 peptide structures selected from a group of peptide structures identified in Table 8,
   wherein the group of peptide structures in Table 8 is associated with the PC disease state; and
   wherein the group of peptide structures is listed in Table 8 with respect to relative significance to the disease indicator; and
generating a diagnosis output based on the disease indicator.

### Embodiment 86

The method of Embodiment 85, wherein the disease indicator comprises a score.

### Embodiment 87

The method of Embodiment 86, wherein generating the diagnosis output comprises:
determining that the score falls above a selected threshold; and
generating the diagnosis output based on the score falling above the selected threshold, wherein the diagnosis output includes a positive diagnosis for the PC disease state.

### Embodiment 88

The method of Embodiment 86, wherein generating the diagnosis output comprises:
determining that the score falls below a selected threshold; and
generating the diagnosis output based on the score falling below the selected threshold, wherein the diagnosis output includes a negative diagnosis for the PC disease state.

### Embodiment 89

The method of Embodiment 87 or Embodiment 88, wherein the score comprises a probability score and the selected threshold is 0.5.

### Embodiment 90

The method of Embodiment 87 or Embodiment 88, wherein the selected threshold falls within a range between 0.4 and 0.6.

### Embodiment 91

The method of any one of Embodiments 85-90, wherein analyzing the peptide structure data comprises:
analyzing the peptide structure data using a binary classification model.

### Embodiment 92

The method of any one of Embodiments 85-91, wherein the at least one peptide structure comprises a glycopeptide structure defined by a peptide sequence and a glycan structure linked to the peptide sequence at a linking site of the peptide sequence, as identified in Table 8, with the peptide sequence being one of SEQ ID NOS: 18, 21, 25, 28, 32, 51-67 as defined in Table 8.

### Embodiment 93

The method of any one of Embodiments 85-92, further comprising:
training the supervised machine learning model using training data,
wherein the training data comprises a plurality of peptide structure profiles for a plurality of subjects and a plurality of subject diagnoses for the plurality of subjects.

### Embodiment 94

The method of Embodiment 93, wherein the plurality of subject diagnoses includes a positive diagnosis for any subject of the plurality of subjects determined to have the PC disease state and a negative diagnosis for any subject of the plurality of subjects determined not to have the PC disease state.

### Embodiment 95

The method of any one of Embodiments 93-94, further comprising:
performing a differential expression analysis using initial training data to compare a first portion of the plurality of subjects diagnosed with the positive diagnosis for the PC disease state versus a second portion of the plurality of subjects diagnosed with the negative diagnosis for the PC disease state; and
identifying a training group of peptide structures based on the differential expression analysis for use as prognostic markers for the PC disease state; and
forming the training data based on the training group of peptide structures identified.

### Embodiment 96

The method of Embodiment 95, wherein training the supervised machine learning model comprises reducing the training group of peptide structures to a final group of peptide structures identified in Table 9.

### Embodiment 97

The method of any one of Embodiments 94-96, wherein the negative diagnosis for the PC disease state indicates a non-pancreatic cancer (PC) state comprising at least one of a healthy state, a benign pancreatitis state, or a control state.

### Embodiment 98

The method of any one of Embodiments 85-97, wherein the supervised machine learning model comprises a logistic regression model.

### Embodiment 99

The method of any one of Embodiments 85-98, wherein the at least 3 peptide structures are included in Table 9, wherein Table 9 identifies a final group of peptide structures that is a subset of the group of peptide structures identified in Table 8.

### Embodiment 100

The method of any one of Embodiments 85-99, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

### Embodiment 101

The method of any one of Embodiments 85-100, wherein the peptide structure data is generated using multiple reaction monitoring mass spectrometry (MRM-MS).

### Embodiment 102

The method of any one of Embodiments 85-101, further comprising:
creating a sample from the biological sample; and
preparing the sample using reduction, alkylation, and enzymatic digestion to form a prepared sample that includes a set of peptide structures.

### Embodiment 103

The method of Embodiment 102, further comprising:
generating the peptide structure data from the prepared sample using multiple reaction monitoring mass spectrometry (MRM-MS).

### Embodiment 104

The method of any one of Embodiments 85-103, wherein generating the diagnosis output comprises:
generating a report identifying that the biological sample evidences the PC disease state.

### Embodiment 105

The method of any one of Embodiments 85-104, further comprising:
generating a treatment output based on at least one of the diagnosis output or the disease indicator.

### Embodiment 106

The method of Embodiment 105, wherein the treatment output comprises at least one of an identification of a treatment to treat the subject or a treatment plan.

### Embodiment 107

The method of Embodiment 106, wherein the treatment comprises at least one of radiation therapy, chemoradiotherapy, surgery, or a targeted drug therapy.

### Embodiment 108

A method of training a model to diagnose a subject with respect to a pancreatic cancer (PC) disease state, the method comprising:
receiving quantification data for a panel of peptide structures for a plurality of subjects,
   wherein the plurality of subjects includes a first portion diagnosed with a negative diagnosis of a PC disease state and a second portion diagnosed with a positive diagnosis of the PC disease state;
   wherein the quantification data comprises a plurality of peptide structure profiles for the plurality of subjects; and
training a machine learning model using the quantification data to diagnose a biological sample with respect to the PC disease state using a group of peptide structures associated with the PC disease state,
   wherein the group of peptide structures is identified in Table 8; and
   wherein the group of peptide structures is listed in Table 8 with respect to relative significance to diagnosing the biological sample.

### Embodiment 109

The method of Embodiment 108, wherein the machine learning model comprises a logistic regression model.

### Embodiment 110

The method of Embodiment 109, wherein the logistic regression model comprises a LASSO regression model.

### Embodiment 111

The method of any one of Embodiments 108-110, wherein training the machine learning model comprises:
training the machine learning using a portion of the quantification data corresponding to a training group of peptide structures included in the plurality of peptide structures.

### Embodiment 112

The method of Embodiment 111, further comprising:
performing a differential expression analysis using the quantification data for the plurality of subjects.

### Embodiment 113

The method of Embodiment 112, further comprising:
identifying the training group of peptide structures based on the differential expression analysis, wherein the training group of peptide structures is a subset of the plurality of peptide structures that has been determined to be relevant to diagnosing the PC disease state.

### Embodiment 114

The method of Embodiment 113, wherein training the machine learning model comprises reducing the training group of peptide structures to a final group of peptide structures identified in Table 9.

### Embodiment 115

The method of any one of Embodiments 108-114, wherein the negative diagnosis for the PC state indicates a non-pancreatic cancer (PC) state comprising at least one of a healthy state, a benign pancreatitis state, or a control state.

### Embodiment 116

The method of any one of Embodiments 108-115, wherein the quantification data for the panel of peptide structures for the plurality of subjects diagnosed with the plurality of PC disease states comprises at least one of an abundance, a relative abundance, a normalized abundance, a relative quantity, an adjusted quantity, a normalized quantity, a relative concentration, an adjusted concentration, or a normalized concentration.

### Embodiment 117

A method of monitoring a subject for a pancreatic cancer (PC) disease state, the method comprising:
receiving first peptide structure data for a first biological sample obtained from a subject at a first timepoint;
analyzing the first peptide structure data using a supervised machine learning model to generate a first disease indicator based on at least 3 peptide structures selected from a group of peptide structures identified in Table 8, wherein the group of peptide structures in Table 8 comprises a group of peptide structures associated with a PC disease state;
receiving second peptide structure data of a second biological sample obtained from the subject at a second timepoint;
analyzing the second peptide structure data using the supervised machine learning model to generate a second disease indicator based on the at least 3 peptide structures selected from the group of peptide structures identified in Table 8; and
generating a diagnosis output based on the first disease indicator and the second disease indicator.

### Embodiment 118

The method of Embodiment 117, wherein the at least 3 peptide structures are included in Table 9, wherein Table 9 identifies a final group of peptide structures that is a subset of the group of peptide structures in Table 8.

### Embodiment 119

The method of Embodiment 117 or Embodiment 118, wherein generating the diagnosis output comprises:
comparing the second disease indicator to the first disease indicator.

### Embodiment 120

The method of any one of Embodiments 117-119, wherein the first disease indicator indicates that the first biological sample evidences a negative diagnosis for the PC disease state and the second biological sample evidences a positive diagnosis for the PC disease state.

### Embodiment 121

The method of any one of Embodiments 117-120, wherein the diagnosis output identifies whether a non-PC disease state has progressed to the PC disease state, wherein the non-PC disease state includes either a healthy state or a benign pancreatitis state.

### Embodiment 122

The method of any one of Embodiments 117-121, wherein the supervised machine learning model comprises a logistic regression model.

### Embodiment 123

A composition comprising at least one of peptide structures PS-1 to PS-22 identified in Table 8.

### Embodiment 124

A composition comprising at least the peptide structure of IGG1_297_3510 identified in Table 1 and 8.

### Embodiment 125

A composition comprising a peptide structure or a product ion, wherein:
the peptide structure or the product ion comprises an amino acid sequence having at least 90% sequence identity to any one of SEQ ID NOS: 18, 21, 25, 28, 32, 51-67, corresponding to peptide structures PS-1 to PS-22 in Table 8; and
the product ion is selected as one from a group consisting of product ions identified in Table 10 including product ions falling within an identified m/z range.

### Embodiment 126

A composition comprising a glycopeptide structure selected as one from a group consisting of peptide structures PS-1 to PS-22 identified in Table 8, wherein:
the glycopeptide structure comprises:
   an amino acid peptide sequence identified in Table 11 as corresponding to the glycopeptide structure; and
   a glycan structure identified in Table 13 as corresponding to the glycopeptide structure in which the glycan structure is linked to a residue of the amino acid peptide sequence at a corresponding position identified in Table 8; and
      wherein the glycan structure has a glycan composition.

### Embodiment 127

The composition of Embodiment 126, wherein the glycan composition is identified in Table 13.

### Embodiment 128

The composition of Embodiment 126, wherein:
the glycopeptide structure has a precursor ion having a charge identified in Table 10 as corresponding to the glycopeptide structure.

### Embodiment 129

The composition of Embodiment 126, wherein:
the glycopeptide structure has a precursor ion with an m/z ratio within ±1.5 of the m/z ratio listed for the precursor ion in Table 10 as corresponding to the glycopeptide structure.

### Embodiment 130

The composition of Embodiment 126, wherein:
the glycopeptide structure has a precursor ion with an m/z ratio within ±1.0 of the m/z ratio listed for the precursor ion in Table 10 as corresponding to the glycopeptide structure.

### Embodiment 131

The composition of Embodiment 126, wherein:
the glycopeptide structure has a precursor ion with an m/z ratio within ±0.5 of the m/z ratio listed for the precursor ion in Table 10 as corresponding to the glycopeptide structure.

### Embodiment 132

The composition of Embodiment 126, wherein:
the glycopeptide structure has a product ion with an m/z ratio within ±1.0 of the m/z ratio listed for the product ion in Table 10 as corresponding to the glycopeptide structure.

### Embodiment 133

The composition of any one of Embodiments 126-132, wherein:
the glycopeptide structure has a product ion with an m/z ratio within ±0.8 of the m/z ratio listed for the product ion in Table 10 as corresponding to the glycopeptide structure.

### Embodiment 134

The composition of any one of Embodiments 126-133, wherein:
the glycopeptide structure has a product ion with an m/z ratio within ±0.5 of the m/z ratio listed for the product ion in Table 10 as corresponding to the glycopeptide structure.

### Embodiment 135

The composition of any one of Embodiments 126-134, wherein the glycopeptide structure has a monoisotopic mass identified in Table 8 as corresponding to the glycopeptide structure.

### Embodiment 136

A composition comprising a peptide structure selected as one from a plurality of peptide structures identified in Table 8, wherein:
the peptide structure has a monoisotopic mass identified as corresponding to the peptide structure in Table 8; and
the peptide structure comprises the amino acid sequence of SEQ ID NOs: 18, 21, 25, 28, 32, 51-67 identified in Table 18 as corresponding to the peptide structure.

### Embodiment 137

The composition of Embodiment 136, wherein:
the peptide structure has a precursor ion having a charge identified in Table 10 as corresponding to the peptide structure.

### Embodiment 138

The composition of Embodiment 136 or Embodiment 137, wherein:
the peptide structure has a precursor ion with an m/z ratio within ±1.5 of the m/z ratio listed for the precursor ion in Table 10 as corresponding to the peptide structure.

### Embodiment 139

The composition of Embodiment 136 or Embodiment 137, wherein:
the peptide structure has a precursor ion with an m/z ratio within ±1.0 of the m/z ratio listed for the precursor ion in Table 10 as corresponding to the peptide structure.

### Embodiment 140

The composition of Embodiment 136 or Embodiment 137, wherein:
the peptide structure has a precursor ion with an m/z ratio within ±0.5 of the m/z ratio listed for the precursor ion in Table 10 as corresponding to the peptide structure.

### Embodiment 141

The composition of any one of Embodiments 136-140, wherein:
the peptide structure has a product ion with an m/z ratio within ±1.0 of the m/z ratio listed for the product ion in Table 10 as corresponding to the peptide structure.

### Embodiment 142

The composition of any one of Embodiments 136-140, wherein:
the peptide structure has a product ion with an m/z ratio within ±0.8 of the m/z ratio listed for the product ion in Table 10 as corresponding to the peptide structure.

### Embodiment 143

The composition of any one of Embodiments 136-140, wherein:
the peptide structure has a product ion with an m/z ratio within ±0.5 of the m/z ratio listed for the product ion in Table 10 as corresponding to the peptide structure.

### Embodiment 144

A kit comprising at least one agent for quantifying at least one peptide structure identified in Table 8 to carry out part or all of the method of any one of Embodiments 85-122.

### Embodiment 145

A kit comprising at least one agent for quantifying at least one peptide structure identified in Table 9 to carry out part or all of the method of any one of Embodiments 85-122.

### Embodiment 146

A kit comprising at least one of a glycopeptide standard, a buffer, or a set of peptide sequences to carry out part or all of the method of any one of Embodiments 85-122, a peptide sequence of the set of peptide sequences identified by a corresponding one of SEQ ID NOS: 18, 21, 25, 28, 32, 51-67, defined in Table 8.

### Embodiment 147

A system comprising:
one or more data processors; and
a non-transitory computer readable storage medium containing instructions which, when executed on the one or more data processors, cause the one or more data processors to perform part or all of any one of Embodiments 85-122.

### Embodiment 148

A computer-program product tangibly embodied in a non-transitory machine-readable storage medium, including instructions configured to cause one or more data processors to perform part or all of any one of Embodiments 85-122.

### Embodiment 149

A composition comprising a peptide structure or a product ion, wherein:
the peptide structure or the product ion comprises an amino acid sequence having at least 90% sequence identity to any one of SEQ ID NOS: 18, 21, 25, 28, 32, 51-67; and
the product ion is selected as one from a group consisting of product ions identified in Table 10 including product ions falling within an identified m/z range.

### Embodiment 150

A composition comprising a glycopeptide structure selected as one from a group consisting of peptide structures PS-1 to PS-22 identified in Table 8, wherein:
the glycopeptide structure comprises:
   an amino acid peptide sequence identified in Table 11 as corresponding to the glycopeptide structure; and
   a glycan structure identified in Table 6 as corresponding to the glycopeptide structure in which the glycan structure is linked to a residue of the amino acid peptide sequence at a corresponding position identified in Table 8; and
      wherein the glycan structure has a glycan composition.

### Embodiment 151

The composition of Embodiment 150, wherein the glycan composition is identified in Table 13.

### Embodiment 152

The composition of Embodiment 150 or Embodiment 151, wherein:
the glycopeptide structure has a precursor ion having a charge identified in Table 10 as corresponding to the glycopeptide structure.

### Embodiment 153

The composition of any one of Embodiments 150-152, wherein:
the glycopeptide structure has a precursor ion with an m/z ratio within ±1.5 of the m/z ratio listed for the precursor ion in Table 10 as corresponding to the glycopeptide structure.

### Embodiment 154

The composition of any one of Embodiments 150-153, wherein:
the glycopeptide structure has a precursor ion with an m/z ratio within ±1.0 of the m/z ratio listed for the precursor ion in Table 10 as corresponding to the glycopeptide structure.

### Embodiment 155

The composition of any one of Embodiments 150-155, wherein:
the glycopeptide structure has a precursor ion with an m/z ratio within ±0.5 of the m/z ratio listed for the precursor ion in Table 10 as corresponding to the glycopeptide structure.

### Embodiment 156

The composition of any one of Embodiments 150-155, wherein:
the glycopeptide structure has a product ion with an m/z ratio within ±1.0 of the m/z ratio listed for the product ion in Table 10 as corresponding to the glycopeptide structure.

### Embodiment 157

The composition of any one of Embodiments 150-155, wherein:
the glycopeptide structure has a product ion with an m/z ratio within ±0.8 of the m/z ratio listed for the product ion in Table 10 as corresponding to the glycopeptide structure.

### Embodiment 158

The composition of any one of Embodiments 150-155, wherein:
the glycopeptide structure has a product ion with an m/z ratio within ±0.5 of the m/z ratio listed for the product ion in Table 10 as corresponding to the glycopeptide structure.

### Embodiment 159

The composition of any one of Embodiments 150-158, wherein the glycopeptide structure has a monoisotopic mass identified in Table 8 as corresponding to the glycopeptide structure.

### Embodiment 160

A composition comprising a peptide structure selected as one of PS-1 to PS-22 peptide structures identified in Table 8, wherein:
the peptide structure has a monoisotopic mass identified as corresponding to the peptide structure in Table 8; and
the peptide structure comprises the amino acid sequence of SEQ ID NOS: 18, 21, 25, 28, 32, 51-67 identified in Table 8 as corresponding to the peptide structure.

### Embodiment 161

The composition of Embodiment 160, wherein:
the peptide structure has a precursor ion having a charge identified in Table 10 as corresponding to the peptide structure.

### Embodiment 162

The composition of Embodiment 160 or Embodiment 161, wherein:
the peptide structure has a precursor ion with an m/z ratio within ±1.5 of the m/z ratio listed for the precursor ion in Table 10 as corresponding to the peptide structure.

### Embodiment 163

The composition of Embodiment 160 or Embodiment 161, wherein:
the peptide structure has a precursor ion with an m/z ratio within ±1.0 of the m/z ratio listed for the precursor ion in Table 10 as corresponding to the peptide structure.

### Embodiment 164

The composition of Embodiment 160 or Embodiment 77, wherein:
the peptide structure has a precursor ion with an m/z ratio within ±0.5 of the m/z ratio listed for the precursor ion in Table 10 as corresponding to the peptide structure.

### Embodiment 165

The composition of any one of Embodiments 160-164, wherein:
the peptide structure has a product ion with an m/z ratio within ±1.0 of the m/z ratio listed for the product ion in Table 10 as corresponding to the peptide structure.

### Embodiment 166

The composition of any one of Embodiments 160-164, wherein:
the peptide structure has a product ion with an m/z ratio within ±0.8 of the m/z ratio listed for the product ion in Table 10 as corresponding to the peptide structure.

### Embodiment 167

The composition of any one of Embodiments 160-164, wherein:
the peptide structure has a product ion with an m/z ratio within ±0.5 of the m/z ratio listed for the product ion in Table 10 as corresponding to the peptide structure.

### XIII. Additional Considerations

Any headers and/or sub-headers between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method for diagnosing a subject with respect to a pancreatic cancer (PC) disease state, the method comprising:
receiving peptide structure data corresponding to a biological sample obtained from the subject;
analyzing the peptide structure data using a supervised machine learning model to generate a disease indicator that indicates whether the biological sample evidences a PC disease state based on at least 3 peptide structures selected from a group of peptide structures identified in Table 8,
wherein the group of peptide structures in Table 8 is associated with the PC disease state; and
wherein the group of peptide structures is listed in Table 8 with respect to relative significance to the disease indicator; and
generating a diagnosis output based on the disease indicator.

2. The method of claim 1, wherein the disease indicator comprises a score.

3. The method of claim 2, wherein generating the diagnosis output comprises:
determining that the score falls above a selected threshold; and
generating the diagnosis output based on the score falling above the selected threshold, wherein the diagnosis output includes a positive diagnosis for the PC disease state.

4. The method of claim 2, wherein generating the diagnosis output comprises:
determining that the score falls below a selected threshold; and
generating the diagnosis output based on the score falling below the selected threshold, wherein the diagnosis output includes a negative diagnosis for the PC disease state.

5. The method of claim 3, wherein the score comprises a probability score and the selected threshold is 0.5.

6. The method of claim 3, wherein the selected threshold falls within a range between 0.4 and 0.6.

7. The method of claim 1, wherein analyzing the peptide structure data comprises:
analyzing the peptide structure data using a binary classification model.

8. The method of claim 1, wherein the at least one peptide structure comprises a glycopeptide structure defined by a peptide sequence and a glycan structure linked to the peptide sequence at a linking site of the peptide sequence, as identified in Table 8, with the peptide sequence being one of SEQ ID NOS: 18, 21, 25, 28, 32, 51-67 as defined in Table 8.

9. The method of claim 1, further comprising:
training the supervised machine learning model using training data,
wherein the training data comprises a plurality of peptide structure profiles for a plurality of subjects and a plurality of subject diagnoses for the plurality of subjects.

10. The method of claim 9, wherein the plurality of subject diagnoses includes a positive diagnosis for any subject of the plurality of subjects determined to have the PC disease state and a negative diagnosis for any subject of the plurality of subjects determined not to have the PC disease state.

11. The method of claim 9, further comprising:
performing a differential expression analysis using initial training data to compare a first portion of the plurality of subjects diagnosed with the positive diagnosis for the PC disease state versus a second portion of the plurality of subjects diagnosed with the negative diagnosis for the PC disease state; and
identifying a training group of peptide structures based on the differential expression analysis for use as prognostic markers for the PC disease state; and
forming the training data based on the training group of peptide structures identified.

12. The method of claim 11, wherein training the supervised machine learning model comprises reducing the training group of peptide structures to a final group of peptide structures identified in Table 9.

13. The method of claim 10, wherein the negative diagnosis for the PC disease state indicates a non-pancreatic cancer (PC) state comprising at least one of a healthy state, a benign pancreatitis state, or a control state.

14. The method of claim 1, wherein the supervised machine learning model comprises a logistic regression model.

15. The method of claim 1, wherein the at least 3 peptide structures are included in Table 9, wherein Table 9 identifies a final group of peptide structures that is a subset of the group of peptide structures identified in Table 8.

16. The method of claim 1, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

17. The method of claim 1, wherein the peptide structure data is generated using multiple reaction monitoring mass spectrometry (MRM-MS).

18. The method of claim 1, further comprising:
creating a sample from the biological sample; and
preparing the sample using reduction, alkylation, and enzymatic digestion to form a prepared sample that includes a set of peptide structures.

19. The method of claim 18, further comprising:
generating the peptide structure data from the prepared sample using multiple reaction monitoring mass spectrometry (MRM-MS).

20. The method of claim 1, wherein generating the diagnosis output comprises:
generating a report identifying that the biological sample evidences the PC disease state.

21. The method of claim 1, further comprising:
generating a treatment output based on at least one of the diagnosis output or the disease indicator.

22. The method of claim 20, wherein the treatment output comprises at least one of an identification of a treatment to treat the subject or a treatment plan.

23. The method of claim 21, wherein the treatment comprises at least one of radiation therapy, chemoradiotherapy, surgery, or a targeted drug therapy.

24. A method of training a model to diagnose a subject with respect to a pancreatic cancer (PC) disease state, the method comprising:
receiving quantification data for a panel of peptide structures for a plurality of subjects,
wherein the plurality of subjects includes a first portion diagnosed with a negative diagnosis of a PC disease state and a second portion diagnosed with a positive diagnosis of the PC disease state;
wherein the quantification data comprises a plurality of peptide structure profiles for the plurality of subjects; and
training a machine learning model using the quantification data to diagnose a biological sample with respect to the PC disease state using a group of peptide structures associated with the PC disease state,
wherein the group of peptide structures is identified in Table 8; and
wherein the group of peptide structures is listed in Table 8 with respect to relative significance to diagnosing the biological sample.

25. The method of claim 24, wherein the machine learning model comprises a logistic regression model.

26. The method of claim 25, wherein the logistic regression model comprises a LASSO regression model.

27. The method of claim 23, wherein training the machine learning model comprises:
training the machine learning using a portion of the quantification data corresponding to a training group of peptide structures included in the plurality of peptide structures.

28. The method of claim 27, further comprising:
performing a differential expression analysis using the quantification data for the plurality of subjects.

29. The method of claim 28, further comprising:
identifying the training group of peptide structures based on the differential expression analysis, wherein the training group of peptide structures is a subset of the plurality of peptide structures that has been determined to be relevant to diagnosing the PC disease state.

30. The method of claim 29, wherein training the machine learning model comprises reducing the training group of peptide structures to a final group of peptide structures identified in Table 9.

31. The method of claim 24, wherein the negative diagnosis for the PC state indicates a non-pancreatic cancer (PC) state comprising at least one of a healthy state, a benign pancreatitis state, or a control state.

32. The method of claim 24, wherein the quantification data for the panel of peptide structures for the plurality of subjects diagnosed with the plurality of PC disease states comprises at least one of an abundance, a relative abundance, a normalized abundance, a relative quantity, an adjusted quantity, a normalized quantity, a relative concentration, an adjusted concentration, or a normalized concentration.

33. A method of monitoring a subject for a pancreatic cancer (PC) disease state, the method comprising:
receiving first peptide structure data for a first biological sample obtained from a subject at a first timepoint;
analyzing the first peptide structure data using a supervised machine learning model to generate a first disease indicator based on at least 3 peptide structures selected from a group of peptide structures identified in Table 8, wherein the group of peptide structures in Table 8 comprises a group of peptide structures associated with a PC disease state;
receiving second peptide structure data of a second biological sample obtained from the subject at a second timepoint;
analyzing the second peptide structure data using the supervised machine learning model to generate a second disease indicator based on the at least 3 peptide structures selected from the group of peptide structures identified in Table 8; and
generating a diagnosis output based on the first disease indicator and the second disease indicator.

34. The method of claim 33, wherein the at least 3 peptide structures are included in Table 9, wherein Table 9 identifies a final group of peptide structures that is a subset of the group of peptide structures in Table 8.

35. The method of claim 33, wherein generating the diagnosis output comprises:
comparing the second disease indicator to the first disease indicator.

36. The method of claim 33, wherein the first disease indicator indicates that the first biological sample evidences a negative diagnosis for the PC disease state and the second biological sample evidences a positive diagnosis for the PC disease state.

37. The method of claim 33, wherein the diagnosis output identifies whether a non-PC disease state has progressed to the PC disease state, wherein the non-PC disease state includes either a healthy state or a benign pancreatitis state.

38. The method of claim 33, wherein the supervised machine learning model comprises a logistic regression model.

39. A composition comprising at least one of peptide structures PS-1 to PS-22 identified in Table 8.

40. A composition comprising at least the peptide structure of IGG1_297_3510 identified in Table 1 and 8.

41. A composition comprising a peptide structure or a product ion, wherein:
the peptide structure or the product ion comprises an amino acid sequence having at least 90% sequence identity to any one of SEQ ID NOS: 18, 21, 25, 28, 32, 51-67, corresponding to peptide structures PS-1 to PS-22 in Table 8; and
the product ion is selected as one from a group consisting of product ions identified in Table 10 including product ions falling within an identified m/z range.

42. A composition comprising a glycopeptide structure selected as one from a group consisting of peptide structures PS-1 to PS-22 identified in Table 8, wherein:
the glycopeptide structure comprises:
an amino acid peptide sequence identified in Table 11 as corresponding to the glycopeptide structure; and
a glycan structure identified in Table 13 as corresponding to the glycopeptide structure in which the glycan structure is linked to a residue of the amino acid peptide sequence at a corresponding position identified in Table 8; and
wherein the glycan structure has a glycan composition.

43. The composition of claim 42, wherein the glycan composition is identified in Table 13.

44. The composition of claim 42, wherein:
the glycopeptide structure has a precursor ion having a charge identified in Table 10 as corresponding to the glycopeptide structure.

45. The composition of claim 42, wherein:
the glycopeptide structure has a precursor ion with an m/z ratio within ±1.5 of the m/z ratio listed for the precursor ion in Table 10 as corresponding to the glycopeptide structure.

46. The composition of claim 42, wherein:
the glycopeptide structure has a precursor ion with an m/z ratio within ±1.0 of the m/z ratio listed for the precursor ion in Table 10 as corresponding to the glycopeptide structure.

47. The composition of claim 42, wherein:
the glycopeptide structure has a precursor ion with an m/z ratio within ±0.5 of the m/z ratio listed for the precursor ion in Table 10 as corresponding to the glycopeptide structure.

48. The composition of claim 42, wherein:
the glycopeptide structure has a product ion with an m/z ratio within ±1.0 of the m/z ratio listed for the product ion in Table 10 as corresponding to the glycopeptide structure.

49. The composition of claim 42, wherein:
the glycopeptide structure has a product ion with an m/z ratio within ±0.8 of the m/z ratio listed for the product ion in Table 10 as corresponding to the glycopeptide structure.

50. The composition of claim 42, wherein:
the glycopeptide structure has a product ion with an m/z ratio within ±0.5 of the m/z ratio listed for the product ion in Table 10 as corresponding to the glycopeptide structure.

51. The composition of claim 42, wherein the glycopeptide structure has a monoisotopic mass identified in Table 8 as corresponding to the glycopeptide structure.

52. A composition comprising a peptide structure selected as one from a plurality of peptide structures identified in Table 8, wherein:
the peptide structure has a monoisotopic mass identified as corresponding to the peptide structure in Table 8; and
the peptide structure comprises the amino acid sequence of SEQ ID NOs: 18, 21, 25, 28, 32, 51-67 identified in Table 18 as corresponding to the peptide structure.

53. The composition of claim 52, wherein:
the peptide structure has a precursor ion having a charge identified in Table 10 as corresponding to the peptide structure.

54. The composition of claim 52, wherein:
the peptide structure has a precursor ion with an m/z ratio within ±1.5 of the m/z ratio listed for the precursor ion in Table 10 as corresponding to the peptide structure.

55. The composition of claim 52, wherein:
the peptide structure has a precursor ion with an m/z ratio within ±1.0 of the m/z ratio listed for the precursor ion in Table 10 as corresponding to the peptide structure.

56. The composition of claim 52, wherein:
the peptide structure has a precursor ion with an m/z ratio within ±0.5 of the m/z ratio listed for the precursor ion in Table 10 as corresponding to the peptide structure.

57. The composition of claim 52, wherein:
the peptide structure has a product ion with an m/z ratio within ±1.0 of the m/z ratio listed for the product ion in Table 10 as corresponding to the peptide structure.

58. The composition of claim 52, wherein:
the peptide structure has a product ion with an m/z ratio within ±0.8 of the m/z ratio listed for the product ion in Table 10 as corresponding to the peptide structure.

59. The composition of claim 52, wherein:
the peptide structure has a product ion with an m/z ratio within ±0.5 of the m/z ratio listed for the product ion in Table 10 as corresponding to the peptide structure.

60. A kit comprising at least one agent for quantifying at least one peptide structure identified in Table 8 to carry out part or all of the method of claim 1.

61. A kit comprising at least one agent for quantifying at least one peptide structure identified in Table 9 to carry out part or all of the method of claim 1.

62. A kit comprising at least one of a glycopeptide standard, a buffer, or a set of peptide sequences to carry out part or all of the method of claim 1, a peptide sequence of the set of peptide sequences identified by a corresponding one of SEQ ID NOS: 18, 21, 25, 28, 32, 51-67, defined in Table 8.

63. A system comprising:
one or more data processors; and
a non-transitory computer readable storage medium containing instructions which, when executed on the one or more data processors, cause the one or more data processors to perform part or all of claim 1.

64. A computer-program product tangibly embodied in a non-transitory machine-readable storage medium, including instructions configured to cause one or more data processors to perform part or all of claim 1.

65. A composition comprising a peptide structure or a product ion, wherein:
the peptide structure or the product ion comprises an amino acid sequence having at least 90% sequence identity to any one of SEQ ID NOS: 18, 21, 25, 28, 32, 51-67; and
the product ion is selected as one from a group consisting of product ions identified in Table 10 including product ions falling within an identified m/z range.

66. A composition comprising a glycopeptide structure selected as one from a group consisting of peptide structures PS-1 to PS-22 identified in Table 8, wherein:
the glycopeptide structure comprises:
an amino acid peptide sequence identified in Table 11 as corresponding to the glycopeptide structure; and
a glycan structure identified in Table 6 as corresponding to the glycopeptide structure in which the glycan structure is linked to a residue of the amino acid peptide sequence at a corresponding position identified in Table 8; and
wherein the glycan structure has a glycan composition.

67. The composition of claim 66, wherein the glycan composition is identified in Table 13.

68. The composition of claim 66, wherein:
the glycopeptide structure has a precursor ion having a charge identified in Table 10 as corresponding to the glycopeptide structure.

69. The composition of claim 66, wherein:
the glycopeptide structure has a precursor ion with an m/z ratio within ±1.5 of the m/z ratio listed for the precursor ion in Table 10 as corresponding to the glycopeptide structure.

70. The composition of claim 66, wherein:
the glycopeptide structure has a precursor ion with an m/z ratio within ±1.0 of the m/z ratio listed for the precursor ion in Table 10 as corresponding to the glycopeptide structure.

71. The composition of claim 66, wherein:
the glycopeptide structure has a precursor ion with an m/z ratio within ±0.5 of the m/z ratio listed for the precursor ion in Table 10 as corresponding to the glycopeptide structure.

72. The composition of claim 66, wherein:
the glycopeptide structure has a product ion with an m/z ratio within ±1.0 of the m/z ratio listed for the product ion in Table 10 as corresponding to the glycopeptide structure.

73. The composition of claim 66, wherein:
the glycopeptide structure has a product ion with an m/z ratio within ±0.8 of the m/z ratio listed for the product ion in Table 10 as corresponding to the glycopeptide structure.

74. The composition of claim 66, wherein:
the glycopeptide structure has a product ion with an m/z ratio within ±0.5 of the m/z ratio listed for the product ion in Table 10 as corresponding to the glycopeptide structure.

75. The composition of claim 66, wherein the glycopeptide structure has a monoisotopic mass identified in Table 8 as corresponding to the glycopeptide structure.

76. A composition comprising a peptide structure selected as one of PS-1 to PS-22 peptide structures identified in Table 8, wherein:
the peptide structure has a monoisotopic mass identified as corresponding to the peptide structure in Table 8; and
the peptide structure comprises the amino acid sequence of SEQ ID NOS: 18, 21, 25, 28, 32, 51-67 identified in Table 8 as corresponding to the peptide structure.

77. The composition of claim 76, wherein:
the peptide structure has a precursor ion having a charge identified in Table 10 as corresponding to the peptide structure.

78. The composition of claim 76, wherein:
the peptide structure has a precursor ion with an m/z ratio within ±1.5 of the m/z ratio listed for the precursor ion in Table 10 as corresponding to the peptide structure.

79. The composition of claim 76, wherein:
the peptide structure has a precursor ion with an m/z ratio within ±1.0 of the m/z ratio listed for the precursor ion in Table 10 as corresponding to the peptide structure.

80. The composition of claim 76, wherein:
the peptide structure has a precursor ion with an m/z ratio within ±0.5 of the m/z ratio listed for the precursor ion in Table 10 as corresponding to the peptide structure.

81. The composition of claim 77, wherein:
the peptide structure has a product ion with an m/z ratio within ±1.0 of the m/z ratio listed for the product ion in Table 10 as corresponding to the peptide structure.

82. The composition of claim 77, wherein:
the peptide structure has a product ion with an m/z ratio within ±0.8 of the m/z ratio listed for the product ion in Table 10 as corresponding to the peptide structure.

83. The composition of claim 77, wherein:
the peptide structure has a product ion with an m/z ratio within ±0.5 of the m/z ratio listed for the product ion in Table 10 as corresponding to the peptide structure.


SEQUENCE LISTING (69 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 3 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 4 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 5 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 6 - protein, 201 aa, Homo sapiens
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 7 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 8 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 9 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 10 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 11 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 12 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 13 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 14 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 15 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 16 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 17 - protein, 584 aa, Homo sapiens
MFAVVFFILSLMTCQPGVTAQEKVNQRVRRAATPAAVTCQLSNWSEWTDCFPCQDKKYRHRSLLQPNKFGGTICSGDIWDQASCSSSTTCVRQAQCGQDFQCKETGRCLKRHLVCNGDQDCLDGSDEDDCEDVRAIDEDCSQYEPIPGSQKAALGYNILTQEDAQSVYDASYYGGQCETVYNGEWRELRYDSTCERLYYGDDEKYFRKPYNFLKYHFEALADTGISSEFYDNANDLLSKVKKDKSDSFGVTIGIGPAGSPLLVGVGVSHSQDTSFLNELNKYNEKKFIFTRIFTKVQTAHFKMRKDDIMLDEGMLQSLMELPDQYNYGMYAKFINDYGTHYITSGSMGGIYEYILVIDKAKMESLGITSRDITTCFGGSLGIQYEDKINVGGGLSGDHCKKFGGGKTERARKAMAVEDIISRVRGGSSGWSGGLAQNRSTITYRSWGRSLKYNPVVIDFEMQPIHEVLRHTSLGPLEAKRQNLRRALDQYLMEFNACRCGPCFNNGVPILEGTSCRCQCRLGSLGAACEQTQTEGAKADGSWSCWSSWSVCRAGIQERRRECDNPAPQNGGASCPGRKVQTQAC

SEQ ID NO: 18 - protein, 11 aa, Homo sapiens
DLATVYVDVLK

SEQ ID NO: 19 - protein, 22 aa, Homo sapiens
GCVLLSYLNETVTVSASLESVR

SEQ ID NO: 20 - protein, 19 aa, Homo sapiens
VIDFNCTTSSVSSALANTK

SEQ ID NO: 21 - protein, 13 aa, Homo sapiens
NLFLNHSENATAK

SEQ ID NO: 22 - protein, 24 aa, Homo sapiens
SVQEIQATFFYFTPNKTEDTIFLR

SEQ ID NO: 23 - protein, 32 aa, Homo sapiens
SLGNVNFTVSAEALESQELCGTEVPSVPEHGR

SEQ ID NO: 24 - protein, 8 aa, Homo sapiens
AVLTIDEK

SEQ ID NO: 25 - protein, 16 aa, Homo sapiens
YTGNASALFILPDQDK

SEQ ID NO: 26 - protein, 16 aa, Homo sapiens
VVLHPNYSQVDIGLIK

SEQ ID NO: 27 - protein, 17 aa, Homo sapiens
NGTGHGNSTHHGPEYMR

SEQ ID NO: 28 - protein, 13 aa, Homo sapiens
CGLVPVLAENYNK

SEQ ID NO: 29 - protein, 11 aa, Homo sapiens
ENISDPTSPLR

SEQ ID NO: 30 - protein, 18 aa, Homo sapiens
IPCSQPPQIEHGTINSSR

SEQ ID NO: 31 - protein, 9 aa, Homo sapiens
TPLTANITK

SEQ ID NO: 32 - protein, 9 aa, Homo sapiens
EEQYNSTYR

SEQ ID NO: 33 - protein, 15 aa, Homo sapiens
VCQDCPLLAPLNDTR

SEQ ID NO: 34 - protein, 24 aa, Homo sapiens
MVSHHNLTTGATLINEQWLLTTAK

SEQ ID NO: 35 - protein, 22 aa, Homo sapiens
AALAAFNAQNNGSNFQLEEISR

SEQ ID NO: 36 - protein, 11 aa, Homo sapiens
YKNNSDISSTR

SEQ ID NO: 37 - protein, 15 aa, Homo sapiens
QDQCIYNTTYLNVQR

SEQ ID NO: 38 - protein, 19 aa, Homo sapiens
SLTFNETYQDISELVYGAK

SEQ ID NO: 39 - protein, 8 aa, Homo sapiens
LYYGDDEK

SEQ ID NO: 40 - protein, 7 aa, Homo sapiens
ENGTISR

SEQ ID NO: 41 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 42 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 43 - protein, 330 aa, Homo sapiens [sequence omitted]

SEQ ID NO: 44 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 45 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 46 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 47 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 48 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 49 - protein, 188 aa, Homo sapiens
MFHQIWAALLYFYGIILNSIYQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 50 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 51 - protein, 6 aa, Homo sapiens
NEEYNK

SEQ ID NO: 52 - protein, 10 aa, Homo sapiens
DLLLPQPDLR

SEQ ID NO: 53 - protein, 32 aa, Homo sapiens
ADTHDEILEGLNFNLTEIPEAQIHEGFQELLR

SEQ ID NO: 54 - protein, 21 aa, Homo sapiens
VGQLQLSHNLSLVILVPQNLK

SEQ ID NO: 55 - protein, 8 aa, Homo sapiens
DTFVNASR

SEQ ID NO: 56 - protein, 27 aa, Homo sapiens
LSLHRPALEDLLLGSEANLTCTLTGLR

SEQ ID NO: 57 - protein, 10 aa, Homo sapiens
VNHVTLSQPK

SEQ ID NO: 58 - protein, 16 aa, Homo sapiens
SVQEIQATFFYFTPNK

SEQ ID NO: 59 - protein, 22 aa, Homo sapiens
STGKPTLYNVSLVMSDTAGTCY

SEQ ID NO: 60 - protein, 19 aa, Homo sapiens
VLSNNSDANLELINTWVAK

SEQ ID NO: 61 - protein, 32 aa, Homo sapiens; identical to SEQ ID NO: 53

SEQ ID NO: 62 - protein, 9 aa, Homo sapiens
EEQFNSTFR

SEQ ID NO: 63 - protein, 32 aa, Homo sapiens
NCGVNCSGDVFTALIGEIASPNYPKPYPENSR

SEQ ID NO: 64 - protein, 33 aa, Homo sapiens
IITILEEEMNVSVCGLYTYGKPVPGHVTVSICR

SEQ ID NO: 65 - protein, 14 aa, Homo sapiens
SYTITGLQPGTDYK

SEQ ID NO: 66 - protein, 7 aa, Homo sapiens
AFLLTPR

SEQ ID NO: 67 - protein, 22 aa, Homo sapiens
TSESGELHGLTTEEEFVEGIYK

SEQ ID NO: 68 - protein, 7 aa, synthetic construct
ECDMHYK

SEQ ID NO: 69 - protein, 7 aa, synthetic construct
  SITE 2: note = acetamide-modified Cysteine
ECDMHYK